(19) United States
(12) Patent Application Publication
(10) Pub. No.: US 2009/0221645 A1
(43) Pub. Date: Sep. 3, 2009

(54) Dihydroimidazothiazole Derivatives

(76) Inventors: Oscar Barba, Oxford (GB); Graham John Dawson, Oxford (GB); Thomas Martin Krulle, Oxford (GB); Robert John Rowley, Oxford (GB); Donald Smyth, Oxford (GB); Gerard Hugh Thomas, Oxford (GB)

Correspondence Address: OSI PHARMACEUTICALS, INC., 41 PINELAWN ROAD, MELVILLE, NY, 11747, US

(21) Appl. No.: 11/884,087

(22) PCT Filed: Feb. 8, 2006
(86) PCT No.: PCT/GB06/50031

§ 371 Date: Nov. 17, 2008

Related U.S. Application Data
(60) Provisional application No. 60/650,906, filed on Feb. 8, 2005.
(60) Provisional application No. 60/684,369, filed on May 25, 2005.
(60) Provisional application No. 60/761,666, filed on Jan. 24, 2006.

(51) Int. Cl. A61K 31/429; C07D 513/04; A61K 31/4439; A61P 3/00; A61P 3/04

(52) U.S. Cl. 514/338; 548/154; 546/270.1; 514/368

(57) ABSTRACT

Compounds of formula (I) or pharmaceutically acceptable salts thereof, exhibit 5-HT_{1A} agonism in addition to noradrenaline reuptake inhibition and optionally also 5-HT reuptake inhibition are useful for the treatment of obesity.

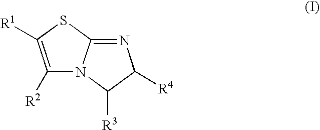

### BACKGROUND OF THE INVENTION

The present invention is directed to dihydroimidazo[2,1-b]thiazole derivatives exhibiting 5-HT_{1A} agonism, in addition to noradrenaline reuptake inhibition and optionally also 5-HT reuptake inhibition, that are useful for the treatment of obesity e.g. as regulators of feeding and/or satiety.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) which is characterized by hypertension and its associated pathologies including atherosclerosis, lipidemia, hyperlipidemia and hypercholesterolemia have been associated with decreased insulin sensitivity which can lead to abnormal blood sugar levels when challenged. Myocardial ischemia and microvascular disease is an established morbidity associated with untreated or poorly controlled metabolic syndrome.

A class of compounds called the Serotonin/Noradrenaline Reuptake Inhibitors (SNRI's) are believed to reduce food intake and increase energy expenditure by enhancing central 5-HT and noradrenaline (NA) function. Sibutramine ((+) and (−) enantiomers of 1-(4-chlorophenyl)-N,N-dimethyl-α-(2-methylpropyl)cyclobutanemethanamine) which is a member of this class of compounds has been shown to produce dose-dependant long lasting weight reduction in obese patients by enhancing natural satiety and increasing energy expenditure by stimulating thermogenesis. The most common side effects associated with Sibutramine therapy include headache, dry mouth, constipation and insomnia. However, it is also associated with dose related increases in heart rate and blood pressure which limit the weight loss that can be achieved and is contraindicated in patients with cardiovascular history.

A SNRI with the addition of 5-HT_{1A} agonist activity is expected to have an improved cardiovascular profile as compared to a SNRI alone, through activation of post-synaptic 5-HT_{1A} receptors thereby reducing sympathetic drive (van den Buuse, M. & Wegener, N., 2005, Eur. J. Pharmacol., Vol. 507(1-3) PP 187-98; Chamienia, A. L. & Johns, E. J. 1996. Brit. J. Pharmacol., Vol. 118(8) PP 1891-1898). 5-HT_{1A} agonists also increase the activity of noradrenergic neurones in the locus coeruleus (Szabo, S. T. & Blier, P., 2001; Eur. J. Neuroscience, Vol. 13, PP 2077-2087) through a reduction in firing of 5-HT neurones in the raphe via 5-HT_{1A} autoreceptor activation thereby removing the 5-HT tonic inhibition in noradrenergic activity throughout the brain (Béïque, J-C., de Montigny, C., Blier, P. & DeBonnel, G. 1999; Synapse, Vol. 32, PP 198-211; Haddjeri, N., de Montigny, C. & Blier, P. 1997 Brit. J. Pharmacol., Vol. 120, PP 865-875). It has been demonstrated that postsynaptic 5-HT_{1A} receptors do not significantly down-regulate after repeated administration of 5-HT_{1A} agonists indicating that there should be no reduction in efficacy with a chronic treatment (Anxiety and the Serotonin1A Receptor, Jeremy D. Coplan, Susan I. Wolk, and Donald F. Klein; Mochizuki, D., Hokonohara, T., Kawasaki, K., & Miki, N. 2002. J. Psychopharmacol., Vol. 16(3) PP 253-260).

WO97/02269 and WO00/71549 disclose condensed thiazole derivatives having 5-HT receptor affinity.

WO98/41528 discloses compounds exhibiting monoamine reuptake inhibition.

WO02/26747 discloses the use of compounds which have dual 5-HT_{1A} agonist/monoamine reuptake inhibitory activity for use in the treatment of obesity.

WO01/62341 discloses a method for the treatment of obesity comprising the administration of a monoamine reuptake inhibitor and a 5-HT_{1A} agonist.

WO01/68653 discloses dihydroimidazo[2,1-b]thiazole and dihydro-5H-thiazolo[3,2-a]pyrimidines which have dual 5-HT_{1A} agonist/monoamine reuptake inhibitory activity for use in the treatment of depression, obesity and other disorders.

Sharpe et al, J. Med. Chem., 1971, 14(10), 977 disclose phenacylthioimidazolines and 3-aryl-5,6-dihydroimidazo[2,1-b]thiazoles, including the compounds 3-naphthalen-1-yl-5,6-dihydroimidazo[2,1-b]thiazole hydrobromide and 3-naphthalen-2-yl-5,6-dihydroimidazo[2,1-b]thiazole hydrobromide, having antidepressant activity. No mechanism of action is disclosed or suggested for these compounds.

USSR Patent Application No. 910637 discloses 3-(3-chloro-4-propoxyphenyl)-5,6-dihydroimidazo[2,1-b]thiazole and 3-(3-chloro-4-butyloxyphenyl)-5,6-dihydroimidazo[2,1-b]thiazole having mutagenic activity.

U.S. Pat. Nos. 3,671,533 and 3,806,515 disclose the 5,6-dihydroimidazo[2,1-b]thiazole derivatives 3-(4-chlorophenyl)-2-ethyl-5,6-dihydroimidazo[2,1-b]thiazole and 3-(4-chlorophenyl)-2-methyl-5,6-dihydroimidazo[2,1-b]thiazole and certain 2,3,5,6-tetrahydroimidazo[2,1-b]thiazole derivatives. 2,3,5,6-Tetrahydroimidazo[2,1-b]thiazole derivatives are stated to be preferred. The compounds are stated to have CNS stimulant activity and may be useful as antidepressants, anorectics and diuretics.

U.S. Pat. No. 3,715,367 discloses the compounds 3-(3,4-dichlorophenyl)-5,6-dihydroimidazo[2,1-b]thiazole, 3-(2-hydroxy-5-methylphenyl)-5,6-dihydroimidazo[2,1-b]thiazole and 3-(4-aminophenyl)-5,6-dihydroimidazo[2,1-b]thiazole having antidepressant activity.

There is a continuing need for novel antiobesity and antidiabetic agents, particularly ones that are well tolerated with few adverse effects.

### SUMMARY OF THE INVENTION

Compounds of formula (I):

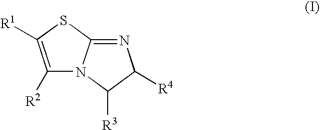

or pharmaceutically acceptable salts thereof, exhibit 5-HT_{1A} agonism in addition to noradrenaline reuptake inhibition and optionally also 5-HT reuptake inhibition and are useful as for the treatment of obesity e.g. as regulators of feeding and/or satiety.

### DETAILED DESCRIPTION OF THE INVENTION

The present invention is directed to a compound of formula (I):

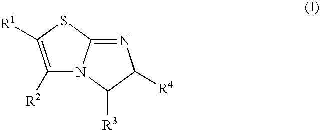

or a pharmaceutically acceptable salt thereof,

wherein R¹ is hydrogen, halo, C₁₋₆ alkyl optionally substituted by one or more halo atoms or hydroxy groups, C₃₋₆ cycloalkyl optionally substituted by one or more halo atoms or hydroxy groups, C₁₋₂ alkylC₃₋₆ cycloalkyl optionally substituted by one or more halo atoms or hydroxy groups, C₁₋₆ alkoxycarbonyl, cyano, —C═N—OR⁷, C₂₋₆ alkenyl optionally substituted by one or more halo atoms or hydroxy groups in which hydroxy is not directly attached to either carbon of the double bond, C₂₋₆ alkynyl optionally substituted by one or more halo atoms or hydroxy groups in which hydroxy is not directly attached to either carbon of the triple bond, (CH₂)ₘNR⁵R⁶, C₁₋₃alkoxy, C₁₋₃ alkylthio, C₁₋₃ alkoxyC₁₋₃ alkyl or C₁₋₃ alkylthioC₁₋₃ alkyl;

R² is an 8- to 10-membered bicyclic aromatic group optionally containing up to 3 heteroatoms selected from N and S, or phenyl, provided that R² is not benzo[b]thiophene;

R² may be optionally substituted by one or more groups selected from halo, cyano, hydroxy, NR⁵R⁶, CONR⁵R⁶, or COOR⁷, or C₁₋₃ alkyl, C₂₋₃ alkenyl, C₁₋₃ alkynyl, C₃₋₆ cycloalkyl, C₁₋₃ alkoxy, C₁₋₃ hydroxyalkyl, C₂₋₃ alkoxyalkyl or C₁₋₃ alkylS(O)ₙ any of which may be optionally substituted by one or more halo atoms; or when R² is phenyl two substituents on phenyl may join to form a fused C₅₋₆ carbocyclic ring;

R³ and R⁴ are independently hydrogen or C₁₋₃ alkyl;

R⁵ and R⁶ are independently hydrogen or C₁₋₃ alkyl, or together with the nitrogen to which they are attached form a 5- or 6-membered heterocyclyl group;

R⁷ is hydrogen or C₁₋₃ alkyl;

m is 1, 2 or 3; and

n is 0, 1 or 2;

provided that the compound is not
a) 3-naphthalen-1-yl-5,6-dihydroimidazo[2,1-b]thiazole hydrobromide,
b) 3-naphthalen-2-yl-5,6-dihydroimidazo[2,1-b]thiazole hydrobromide,
c) 3-(3-chloro-4-propoxyphenyl)-5,6-dihydroimidazo[2,1-b]thiazole,
d) 3-(4-chlorophenyl)-2-ethyl-5,6-dihydroimidazo[2,1-b]thiazole,
e) 3-(4-chlorophenyl)-2-methyl-5,6-dihydroimidazo[2,1-b]thiazole,
f) 3-(3,4-dichlorophenyl)-5,6-dihydroimidazo[2,1-b]thiazole,
g) 3-(2-hydroxy-5-methylphenyl)-5,6-dihydroimidazo[2,1-b]thiazole,
h) 3-(4-aminophenyl)-5,6-dihydroimidazo[2,1-b]thiazole,
i) 3-(2-chlorophenyl)-5,6-dihydroimidazo[2,1-b]thiazole,
j) 3-(3-chlorophenyl)-5,6-dihydroimidazo[2,1-b]thiazole,
k) 3-(4-chlorophenyl)-5,6-dihydroimidazo[2,1-b]thiazole,
l) 3-(2,4-dichlorophenyl)-5,6-dihydroimidazo[2,1-b]thiazole,
m) 3-(2,5-dichlorophenyl)-5,6-dihydroimidazo[2,1-b]thiazole,
n) 3-(4-bromophenyl)-5,6-dihydroimidazo[2,1-b]thiazole,
o) 3-(2,4-difluorophenyl)-5,6-dihydroimidazo[2,1-b]thiazole,
p) 3-(2-methylphenyl)-5,6-dihydroimidazo[2,1-b]thiazole,
q) 3-(3-methylphenyl)-5,6-dihydroimidazo[2,1-b]thiazole,
r) 3-(4-methylphenyl)-5,6-dihydroimidazo[2,1-b]thiazole,
s) 3-(2,4-dimethylphenyl)-5,6-dihydroimidazo[2,1-b]thiazole,
t) 3-(3,4-dimethylphenyl)-5,6-dihydroimidazo[2,1-b]thiazole,
u) 3-(4-cyanophenyl)-5,6-dihydroimidazo[2,1-b]thiazole,
v) 3-(4-carboxyphenyl)-5,6-dihydroimidazo[2,1-b]thiazole,
w) 3-(2-methoxyphenyl)-5,6-dihydroimidazo[2,1-b]thiazole,
x) 3-(3-methoxyphenyl)-5,6-dihydroimidazo[2,1-b]thiazole,
y) 3-(4-methoxyphenyl)-5,6-dihydroimidazo[2,1-b]thiazole,
z) 3-(2-hydroxyphenyl)-5,6-dihydroimidazo[2,1-b]thiazole,
aa) 3-(3-hydroxyphenyl)-5,6-dihydroimidazo[2,1-b]thiazole,
ab) 3-(4-hydroxyphenyl)-5,6-dihydroimidazo[2,1-b]thiazole,
ac) 3-(3,4-dihydroxyphenyl)-5,6-dihydroimidazo[2,1-b]thiazole,
ad) 3-(2-hydroxy-4-methoxyphenyl)-5,6-dihydroimidazo[2,1-b]thiazole,
ae) 3-(3-hydroxy-4-methoxyphenyl)-5,6-dihydroimidazo[2,1-b]thiazole,
af) 3-(4-hydroxy-3-methoxyphenyl)-5,6-dihydroimidazo[2,1-b]thiazole,
ag) 3-(4-hydroxy-3-chlorophenyl)-5,6-dihydroimidazo[2,1-b]thiazole,
ah) 3-(4-hydroxy-3-methylphenyl)-5,6-dihydroimidazo[2,1-b]thiazole, or
ai) 3-(3,4-dichlorophenyl)-2-phenyl-5,6-dihydroimidazo[2,1-b]thiazole.

A preferred group of compounds of the invention are the compounds of formula (Ia):

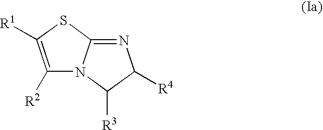

or a pharmaceutically acceptable salt thereof,

wherein R¹ is hydrogen, halo, C₁₋₆ alkyl optionally substituted by one or more halo atoms or hydroxy groups, C₃₋₆ cycloalkyl optionally substituted by one or more halo atoms or hydroxy groups, C₁₋₂ alkylC₃₋₆ cycloalkyl optionally substituted by one or more halo atoms or hydroxy groups, C₁₋₆ alkoxycarbonyl, cyano, —C═N—OR⁷, C₂₋₆ alkenyl optionally substituted by one or more halo atoms or hydroxy groups in which hydroxy is not directly attached to either carbon of the double bond, C₂₋₆ alkynyl optionally substituted by one or more halo atoms or hydroxy groups in which hydroxy is not directly attached to either carbon of the triple bond, (CH₂)ₘNR⁵R⁶, C₁₋₃alkoxy, C₁₋₃ alkylthio, C₁₋₃ alkoxyC₁₋₃ alkyl or C₁₋₃ alkylthioC₁₋₃ alkyl;

R² is an 8- to 10-membered bicyclic aromatic group optionally containing up to 3 heteroatoms selected from N and S, provided that R² is not benzo[b]thiophene;

R² may be optionally substituted by one or more groups selected from halo, cyano, hydroxy, NR⁵R⁶, CONR⁵R⁶, or COOR⁷, or C₁₋₃ alkyl, C₁₋₃ alkoxy, C₁₋₃ hydroxyalkyl, C₂₋₃ alkoxyalkyl or C₁₋₃ alkylS(O)ₙ any of which may be optionally substituted by one or more halo atoms;

R³ and R⁴ are independently hydrogen or C₁₋₃ alkyl;

R⁵ and R⁶ are independently hydrogen or C₁₋₃ alkyl, or together with the nitrogen to which they are attached form a 5- or 6-membered heterocyclyl group;

R⁷ is hydrogen or C₁₋₃ alkyl;

m is 1, 2 or 3; and

n is 0, 1 or 2;

provided that the compound is not:
a) 3-naphthalen-1-yl-5,6-dihydroimidazo[2,1-b]thiazole hydrobromide, or
b) 3-naphthalen-2-yl-5,6-dihydroimidazo[2,1-b]thiazole hydrobromide.

A further group of compounds which may be mentioned are the compounds of formula (Ib):

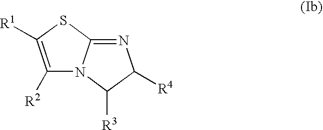

or a pharmaceutically acceptable salt thereof, wherein

R¹ is hydrogen, halo, C₁₋₆ alkyl optionally substituted by one or more halo atoms or hydroxy groups, C₃₋₆ cycloalkyl optionally substituted by one or more halo atoms or hydroxy groups, C₁₋₂ alkylC₃₋₆ cycloalkyl optionally substituted by one or more halo atoms or hydroxy groups, C₁₋₆ alkoxycarbonyl, cyano, —C═N—OR⁷, C₂₋₆ alkenyl optionally substituted by one or more halo atoms or hydroxy groups in which hydroxyl is not directly attached to either carbon of the double bond, C₂₋₆ alkynyl optionally substituted by one or more halo atoms or hydroxy groups in which hydroxyl is not directly attached to either carbon of the triple bond, (CH₂)ₘNR⁵R⁶, C₁₋₃alkoxy, C₁₋₃ alkylthio, C₁₋₃ alkoxyC₁₋₃ alkyl or C₁₋₃ alkylthioC₁₋₃ alkyl;

R² is phenyl substituted by one or more groups selected from halo, cyano, hydroxy, NR⁵R⁶, CONR⁵R⁶, or COOR⁷, or C₁₋₃ alkyl, C₂₋₃ alkenyl, C₂₋₃ alkynyl, C₃₋₆ cycloalkyl, C₁₋₃ alkoxy, C₁₋₃ hydroxyalkyl, C₂₋₃ alkoxyalkyl or C₁₋₃ alkylS(O)ₙ any of which may be optionally substituted by one or more halo atoms;

R³ and R⁴ are independently hydrogen or C₁₋₃ alkyl;

R⁵ and R⁶ are independently hydrogen or C₁₋₃ alkyl, or together with the nitrogen to which they are attached form a 5- or 6-membered heterocyclyl group;

R⁷ is hydrogen or C₁₋₃ alkyl;

m is 1, 2 or 3; and

n is 0, 1 or 2;

provided that the compound is not:
c) 3-(3-chloro-4-propoxyphenyl)-5,6-dihydroimidazo[2,1-b]thiazole,
d) 3-(4-chlorophenyl)-2-ethyl-5,6-dihydroimidazo[2,1-b]thiazole,
e) 3-(4-chlorophenyl)-2-methyl-5,6-dihydroimidazo[2,1-b]thiazole,
f) 3-(3,4-dichlorophenyl)-5,6-dihydroimidazo[2,1-b]thiazole,
g) 3-(2-hydroxy-5-methylphenyl)-5,6-dihydroimidazo[2,1-b]thiazole,
h) 3-(4-aminophenyl)-5,6-dihydroimidazo[2,1-b]thiazole,
i) 3-(2-chlorophenyl)-5,6-dihydroimidazo[2,1-b]thiazole,
j) 3-(3-chlorophenyl)-5,6-dihydroimidazo[2,1-b]thiazole,
k) 3-(4-chlorophenyl)-5,6-dihydroimidazo[2,1-b]thiazole,
l) 3-(2,4-dichlorophenyl)-5,6-dihydroimidazo[2,1-b]thiazole,
m) 3-(2,5-dichlorophenyl)-5,6-dihydroimidazo[2,1-b]thiazole,
n) 3-(4-bromophenyl)-5,6-dihydroimidazo[2,1-b]thiazole,
o) 3-(2,4-difluorophenyl)-5,6-dihydroimidazo[2,1-b]thiazole,
p) 3-(2-methylphenyl)-5,6-dihydroimidazo[2,1-b]thiazole,
q) 3-(3-methylphenyl)-5,6-dihydroimidazo[2,1-b]thiazole,
r) 3-(4-methylphenyl)-5,6-dihydroimidazo[2,1-b]thiazole,
s) 3-(2,4-dimethylphenyl)-5,6-dihydroimidazo[2,1-b]thiazole,
t) 3-(3,4-diethylphenyl)-5,6-dihydroimidazo[2,1-b]thiazole,
u) 3-(4-cyanophenyl)-5,6-dihydroimidazo[2,1-b]thiazole,
v) 3-(4-carboxyphenyl)-5,6-dihydroimidazo[2,1-b]thiazole,
w) 3-(2-methoxyphenyl)-5,6-dihydroimidazo[2,1-b]thiazole,
x) 3-(3-methoxyphenyl)-5,6-dihydroimidazo[2,1-b]thiazole,
y) 3-(4-methoxyphenyl)-5,6-dihydroimidazo[2,1-b]thiazole,
z) 3-(2-hydroxyphenyl)-5,6-dihydroimidazo[2,1-b]thiazole,
aa) 3-(3-hydroxyphenyl)-5,6-dihydroimidazo[2,1-b]thiazole,
ab) 3-(4-hydroxyphenyl)-5,6-dihydroimidazo[2,1-b]thiazole,
ac) 3-(3,4-dihydroxyphenyl)-5,6-dihydroimidazo[2,1-b]thiazole,
ad) 3-(2-hydroxy-4-methoxyphenyl)-5,6-dihydroimidazo[2,1-b]thiazole,
ae) 3-(3-hydroxy-4-methoxyphenyl)-5,6-dihydroimidazo[2,1-b]thiazole,
af) 3-(4-hydroxy-3-methoxyphenyl)-5,6-dihydroimidazo[2,1-b]thiazole,
ag) 3-(4-hydroxy-3-chlorophenyl)-5,6-dihydroimidazo[2,1-b]thiazole,
ah) 3-(4-hydroxy-3-methylphenyl)-5,6-dihydroimidazo[2,1-b]thiazole, or
ai) 3-(3,4-dichlorophenyl)-2-phenyl-5,6-dihydroimidazo[2,1-b]thiazole.

In the compounds of formulae (I), (Ia) and (Ib):

R¹ is preferably hydrogen, C₁₋₆ alkyl optionally substituted by one or more halo atoms or hydroxy groups, C₃₋₆ cycloalkyl optionally substituted by one or more halo atoms or hydroxy groups, or C₁₋₂ alkylC₃₋₆ cycloalkyl optionally substituted by one or more halo atoms or hydroxy groups. R¹ is more preferably C₁₋₆ alkyl, especially methyl.

A further specific group of compounds which may be mentioned are those where R¹ is not hydrogen.

When R¹ is C₁₋₆ alkyl optionally substituted by one or more halo atoms it may be a fluoroalkyl group.

Examples of 8- to 10-membered bicyclic aromatic groups which R² may represent include naphthalenyl, e.g. naphthalen-1-yl or naphthalen-2-yl, thienothiophenyl, e.g. thieno[2,3-b]thiophen-2-yl, indolyl, quinolinyl, e.g. quinolin-2-yl, isoquinolinyl and benzoisothiazole, e.g. benzoisothiaxol-3-yl. R² is preferably naphthalenyl, especially naphthalen-1-yl.

When R² is a substituted 8- to 10-membered bicyclic aromatic group, e.g. naphthalenyl, it is preferably substituted by one or two substituents preferably selected from halo, e.g. fluoro or chloro, and C₁₋₃ alkyl, e.g. methyl. When R² is naphthalen-1-yl it is preferably unsubstituted or substituted in one or two of the 4-, 5- or 7-positions by halo, e.g. fluoro or chloro.

When R² is phenyl it is preferably substituted in the 3-, 4- and/or 5-positions.

When R² is phenyl it is preferably substituted by one or two groups selected from halo and C₁₋₃ alkyl optionally substituted by one or more halo atoms.

When R² is phenyl a specific group of compounds of the invention which may be mentioned are compounds in which, when R¹ is straight chain unsubstituted C₁₋₄ alkyl and R³ and R⁴ are hydrogen, R² is not phenyl substituted only by one, two or three fluoro or chloro atoms in the 3-, 4- and/or 5-positions.

R³ and R⁴ are preferably independently hydrogen or methyl, more preferably R³ and R⁴ are both hydrogen.

The molecular weight of the compounds of formulae (I), (Ia) and (Ib) is preferably less than 800, more preferably less than 600, even more preferably less than 500.

Specific compounds of the invention which may be mentioned are those included in the Examples and pharmaceutically acceptable salts thereof.

As used herein, unless stated otherwise, “alkyl” as well as other groups having the prefix “alk” such as, for example, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃.

The terms “cycloalkyl” and “carbocyclic group” mean carbocycles containing no heteroatoms, and includes monocyclic saturated carbocycles. Examples of cycloalkyl include cyclopropyl, cyclobutyl, cyclopentyl and cyclohexyl.

The term “halo” includes fluorine, chlorine, bromine and iodine atoms.

The term “aryl” includes phenyl and naphthyl, in particular phenyl.

The term “heterocyclyl” includes 5- and 6-membered saturated rings containing one or two nitrogen atoms. Examples of heterocyclyl groups rings include azetidine, pyrrolidine, piperidine and piperazine. Heterocyclyl groups may also contain additional heteroatoms, e.g. morpholine.

Compounds described herein may contain one or more asymmetric centers and may thus give rise to enantiomers, diastereomers and optical isomers. The present invention includes all such possible enantiomers, diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The compounds of the invention may also exhibit atropisomerism, the present invention includes all atropisomers of formula (I) and mixtures thereof.

When a tautomer of the compound of formula (I) exists, the present invention includes any possible tautomers and pharmaceutically acceptable salts thereof, and mixtures thereof, except where specifically drawn or stated otherwise.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, oxalic, pamoic, pantothenic, phosphoric, succinic, sulfuric, tartaric, p-toluenesulfonic acid and the like.

Since the compounds of formula (I) are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below and as summarized in Scheme 1, wherein R¹, R², R³ and R⁴ are hereinbefore defined, R^{x} is hydrogen or C₁₋₆ alkyl and G is a leaving group or hydrogen.

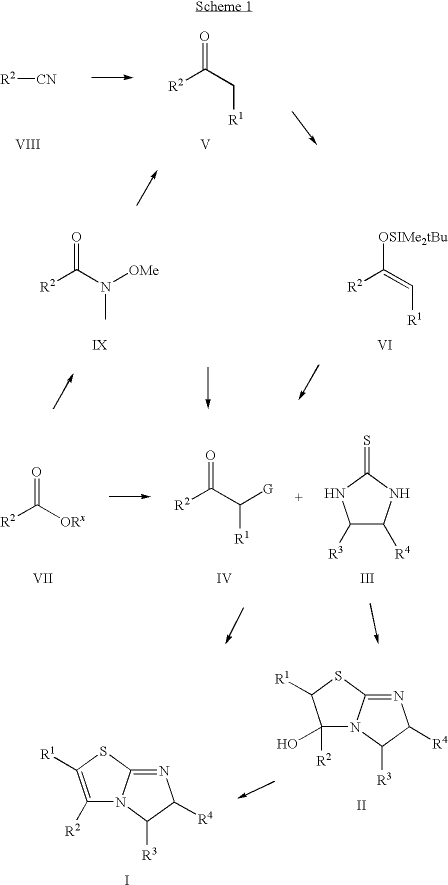

Compounds of formula (I) may be prepared by dehydrating a compound of formula (II):

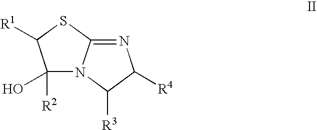

optionally in the presence of an acid, for example acetic or sulfuric acid, at a temperature in the range 0-200° C.; preferably in the range 20-150° C.

Compounds of formula (II) may be prepared by reacting a compound of formula (III):

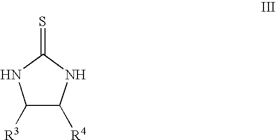

with a compound of formula (IV):

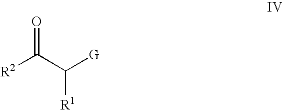

in which G is a leaving group, for example, halo such as bromo or chloro, at a temperature in the range 0-150° C., in the presence of a solvent such as ethanol or acetone, preferably ethanol, in the presence of an acid such as acetic acid; preferably by heating at a temperature in the range 20-120° C.

Compounds of formula (I) may also be prepared by reacting a compound of formula (IV) with a compound of formula (III) at a temperature in the range 0-200° C., preferably in the range 20-120° C., optionally in the presence of an acid, for example acetic acid, and optionally in the presence of a solvent, for example ethanol, without isolation of the intermediate of formula (II).

Compounds of formula (I) may also be prepared by reacting a compound of formula (III) with a compound of formula (IV) where G is H, in the presence of a solvent, for example acetic acid, and an acid, for example sulfuric or hydrochloric acid, and optionally in the presence of a second dehydrating agent, for example acetic anhydride, at a temperature in the range 0-200° C., preferably in the range 20-150° C.

According to a further aspect of the invention there is provided a process for the production of a compound of formula (I) which comprises the step of reacting a compound of formula (III):

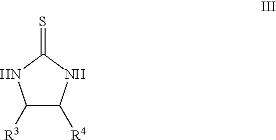

with a compound of formula (IV):

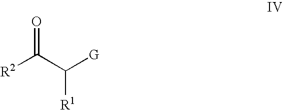

wherein R¹ to R⁴ are as defined for formula (I) and G is hydrogen or a leaving group.

Compounds of formula (I) in which is R¹ is bromo or chloro, may be prepared by reaction of a compound of formula (I) in which R¹ is H with a halogenating agent for example bromine or benzyltrimethylammonium tetrachloroiodate at a temperature in the range of −50-150° C. optionally in the presence of a solvent for example dichloromethane, tetrahydrofuran or acetone.

Compounds of formula (I) in which is R¹ is ethoxycarbonyl may be prepared by reaction of a compound of formula (IV) in which R¹ is ethoxycarbonyl and G is bromo and a compound of formula (III) as described above. Compounds of formula (IV) in which R¹ is ethoxycarbonyl and G is bromo may be prepared from compounds of formula (V) in which R¹ is ethoxycarbonyl by the halogenation methods described below. Compounds of formula (V) in which R¹ is alkoxycarbonyl, e.g. ethoxycarbonyl, may be prepared from compounds of formula (V) in which R¹ is H by reacting with e.g. diethylcarbonate in the presence of a base such as sodium hydride. Compounds of formula (III) are generally commercially available.

Compounds of formula (IV) in which G is halo may be prepared by reaction of a compound of formula (V):

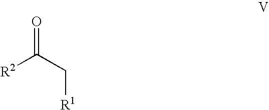

with a halogenating agent, for example a brominating agent such as phenyltrimethylammonium tribromide (PTAT), sodium bromate, bromine or copper(II)bromide in the range 0-200° C. in the presence of a solvent, for example tetrahydrofuran; preferably by heating at a temperature in the range 20-120° C.

Alternatively compounds of formula (IV) in which G is bromo and R² contains basic atoms, e.g. R² is a quinoline or isoquinoline group, may be more suitably prepared by reaction of a compound of formula (V) with pyridinium tribromide in a solvent such as acetic acid at a temperature in the range 20-120° C.

Additionally compounds of formula (V) in which G is halo and R² contains basic atoms, e.g. R² is a quinoline group, may be more suitably prepared by reaction of a compound of formula (V) with tertbutyldimethylsilyl triflate and a base such as triethylamine in a solvent such as dichloromethane in a solvent, such as acetic acid, at a temperature in the range 0-120° C. to give compounds of formula (VI):

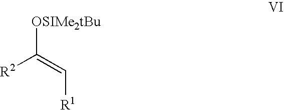

which can then undergo reaction with a halogenating agent, for example a brominating agent such as phenyltrimethylammonium tribromide (PTAT), at a temperature in the range 0-200° C. in the presence of a solvent, for example tetrahydrofuran; preferably by heating at a temperature in the range 20-120° C. to give compounds of formula (IV) after acidic work-up at −78° C. using a mixture of mineral acid e.g. hydrobromic acid and acetic acid.

Compounds of formula (IV) where G is bromo and R¹ is hydrogen may also be prepared by reacting a compound of formula (VII):

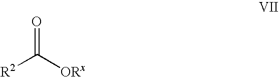

in which R^{x} is C₁₋₆ alkyl, with dibromomethane and an organolithium reagent such as methyllithium in a solvent such as tetrahydrofuran, at a temperature in the range of −78° C. to the boiling point of the chosen solvent, or by using dibromomethane and a base such as lithium diisopropylamine (LDA) in a solvent such as tetrahydrofuran at a temperature in the range of −78° C. to the boiling point of the chosen solvent

Compounds of formula (VII) are generally commercially available.

Compounds of formula (V) may be prepared directly by reacting a compound of formula (VIII):

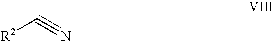

with an organometallic reagent, for example a compound of formula R¹CH₂MgX in which X is halo, for example chloro, in the presence of a solvent, for example tetrahydrofuran or ether, at a temperature in the range of −50° C. to the boiling point of the chosen solvent, followed by hydrolysis of the intermediate imine salt optionally in the presence of an acid, for example hydrochloric acid.

Compounds of formula (VIII) may be prepared by methods known to those skilled in the art or are commercially available.

Additionally compounds of formula (V) in which R¹ is H may also be prepared by reacting a compound of formula (VII) wherein R^{x} is hydrogen with dibromomethane and an organolithium reagent such as methyllithium in a solvent such as tetrahydrofuran at a temperature in the range of −50° C. to the boiling point of the chosen solvent.

Compounds of formula (V) may also be prepared by reacting a compound of formula (IX):

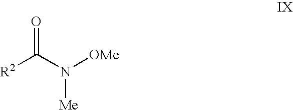

commonly known as a Weinreb amide, with an organometallic reagent, for example a compound of formula R¹CH₂MgX in which X is halo, for example chloro, in the presence of a solvent, for example tetrahydrofuran or ether, at a temperature in the range of −50° C. to the boiling point of the chosen solvent, followed by hydrolysis of the intermediate imine salt optionally in the presence of an acid, for example hydrochloric acid.

Additionally compounds of formula (V) may also be prepared directly from compounds of formula (VII) (where R^{x}═H) by reacting firstly with oxalyl chloride in dichloromethane containing N,N-dimethylformamide (DMF) to give the intermediate acid chloride which is then reacted further with an organometallic reagent, for example a compound of formula R¹CH₂MgX in which X is halo, for example chloro, in the presence of a solvent, for example tetrahydrofuran in the presence of iron (III) acetylacetonate.

Compounds of formula (IX) may be prepared by reacting a compound of formula (VII), where R^{x} is hydrogen, and methoxymethylamine under standard amide coupling conditions known to those skilled in the art.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates as defined above, such as the compounds of formula (II) are also included within the scope of the invention.

Other novel intermediates which are included within the scope of the invention are the compounds of formula (X):

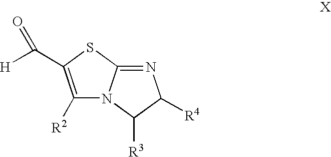

wherein R², R³ and R⁴ are hereinbefore defined. Compounds of formula (X) are useful intermediates for the production of compounds of formula (I) wherein R¹ is e.g. cyano or C₁₋₆ alkyl substituted by hydroxy.

The preferences recited above for the compounds of formulae (I), (Ia) and (Ib) also apply to any intermediate compounds such as those of formulae (II) and (X).

As indicated above the compounds of formula (I) are useful as Noradrenaline and optionally also Serotonin reuptake inhibitors e.g. for the treatment of obesity. For such use the compounds of formula (I) will generally be administered in the form of a pharmaceutical composition.

The invention also encompasses a pharmaceutical composition comprising a compound of formula (I), or a pharmaceutically acceptable salt thereof, in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by inhibiting Noradrenalin and optionally also Serotonin reuptake, e.g. resulting in the treatment of obesity, comprising a pharmaceutically acceptable carrier and a non-toxic therapeutically effective amount of compound of formula (I), including the compounds of provisos a) and b), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 10 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I), including the compounds of provisos a), b) and f) to ai), may be used in the treatment of diseases or conditions in which Noradrenaline and optionally also Serotonin reuptake plays a role. The 5-HT_{1A} agonist activity exhibited by the compounds of formula (I) means that such compounds should provide greater efficacy and lower side effects than a SNRI alone in the treatment of these diseases or conditions.

Thus the invention also provides a method for the treatment of a disease or condition in which Noradrenaline and optionally also Serotonin reuptake plays a role comprising a step of administering to a subject in need thereof an effective amount of a compound of formula (I), including the compounds of provisos a), b) and f) to ai), or a pharmaceutically acceptable salt thereof.

The invention also provides a method for the treatment of a disease or condition in which Noradrenaline and optionally also Serotonin reuptake plays a role and in which 5-HT_{1A} agonism is desirable comprising a step of administering to a subject in need thereof an effective amount of a compound of formula (I), including the compounds of provisos a), b) and f) to ai), or a pharmaceutically acceptable salt thereof.

Diseases or conditions in which Noradrenaline and optionally also Serotonin reuptake plays a role include obesity. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound.

The compounds of the invention may also be used for treating of other diseases in which obesity is a factor including metabolic diseases such as Type II diabetes, metabolic syndrome (syndrome X), impaired glucose tolerance, dyslipidemia, hyperlipidemia, hypertriglyceridemia, hypercholesterolemia, low HDL levels and hypertension.

Other diseases or conditions in which Noradrenaline and optionally also Serotonin reuptake play a role include those described in WO01/68653, for example depression, anxiety, psychoses (e.g. schizophrenia), tardive dyskinesia, drug addiction, drug abuse, cognitive disorders, Alzheimer's disease, obsessive compulsive behaviour, panic attacks, social phobias, eating disorders such as bulimia, anorexia, snacking and binge eating, stress, as an aid to smoking cessation, seizures, neurological disorders such as epilepsy and/or conditions in which there is neurological damage such a stroke, brain trauma, cerebral ischaemia, heads injuries and haemorrhage. Other indications include urinary stress incontinence, neuropathic pain and chronic pain associated with drug therapy or radiation therapy.

The invention also provides a method for the regulation of feeding and/or satiety comprising a step of administering to a subject in need thereof an effective amount of a compound of formula (I), including the compounds of provisos a), b) and f) to ai), or a pharmaceutically acceptable salt thereof.

The invention also provides a method for the treatment of obesity comprising a step of administering to a subject in need thereof an effective amount of a compound of formula (I), including the compounds of provisos a), b) and f) to ai), or a pharmaceutically acceptable salt thereof.

The invention also provides a method for reducing the potential for cardiovascular side effects in the treatment of a disease or condition as defined above comprising a step of administering to a subject in need thereof an effective amount of a compound of formula (I), including the compounds of provisos a), b) and f) to ai), or a pharmaceutically acceptable salt thereof.

The invention also provides a method for the treatment of a metabolic disease selected from Type II diabetes, metabolic syndrome (syndrome X), impaired glucose tolerance, dyslipidemia, hyperlipidemia, hypertriglyceridemia, hypercholesterolemia, low HDL levels and hypertension, comprising a step of administering to a subject in need thereof an effective amount of a compound of formula (I), including the compounds of provisos a), b) and f) to ai), or a pharmaceutically acceptable salt thereof.

The invention also provides the use of a compound of formula (I), including the compounds of provisos a), b) and f) to ai), or a pharmaceutically acceptable salt thereof, in the treatment of a condition as defined above.

The invention also provides the use of a compound of formula (I), including the compounds of provisos a), b) and f) to ai), or a pharmaceutically acceptable salt thereof, in the manufacture of a medicament for the treatment of a condition as defined above.

In the methods of the invention the term “treatment” includes both therapeutic and prophylactic treatment.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I), or a different disease or condition. The therapeutically active compounds may be administered simultaneously, sequentially or separately.

The compounds of formula (I), may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, RXR agonists, fatty acid oxidation inhibitors, α-glucosidase inhibitors, β-agonists, phosphodiesterase inhibitors, lipid lowering agents, glycogen phosphorylase inhibitors, MCH-1 antagonists and CB-1 antagonists, amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, CRF antagonists, CRF binding proteins, thyromimetic compounds, aldose reductase inhibitors, glucocorticoid receptor antagonists, NHE-1 inhibitors or sorbitol dehydrogenase inhibitors.

When used in combination therapy, the compounds of formula (I) are preferably administered in combination with other non-central approaches to obesity e.g. with orlistat (Xenical®) or a with an agonist of GPR119 (GPR119 is identified as SNORF25 in WO00/50562 which discloses both the human and rat receptors and in U.S. Pat. No. 6,468,756 which also discloses the mouse receptor) if peripherally acting.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O—CH₃CN solution, containing 0.1% HCO₂H, over 6 min with UV detection at 220 nm. Gradient information: 0.0-0.3 min 100% H₂O; 0.3-4.25 min: Ramp up to 10% H₂O-90% CH₃CN; 4.25-4.4 min: Ramp up to 100% CH₃CN; 4.4-4.9 min: Hold at 100% CH₃CN; 4.9-5.0 min: Return to 100% H₂O; 5.0-6.0 min: Hold at 100% H₂O. The mass spectra were obtained using an electrospray ionisation source in either the positive (ES⁺) or negative (ES) ion modes. NMR spectra were acquired at 27° C. on a Varian Mercury 400 spectrometer operating at 400 MHz or on a Bruker AMX2 500 spectrometer operating at 500 MHz.

### Abbreviations and Acronyms

Ac: Acetyl; AIBN; 2,2′-Azobisisobutyronitrile; DCM: Dichloromethane; DIPEA: N,N′-Diisopropylethylamine; DMF: N,N-Dimethylformamide; DMSO: Dimethylsulfoxide; EDCI: 1-Ethyl-3-(3′-dimethylaminopropyl)carbodiimide; Et: Ethyl; HOBt: 1-Hydroxybenzotriazole; Me: Methyl; ⁱPr: iso-Propyl; PTAT: Phenyltrimethylammonium tribromide; RT: Retention time; rt: Room temperature; TFA; Trifluoroacetic acid; THF: Tetrahydrofuran.

### Preparation 1

### 2-Bromo-1-naphthalen-1-ylethanone

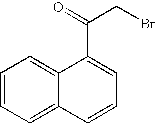

To a stirred solution of 1′-acetonaphthone (0.452 g, 2.66 mmol) in THF (20 mL) was added PTAT (1.10 g, 2.92 mmol) and the reaction was stirred at rt for 16 hr. The reaction mixture was filtered and the solid washed with THF (2×20 mL). The filtrate was concentrated in vacuo and the residue partitioned between water (30 mL) and DCM (2×30 mL). The combined organics were dried (MgSO₄), concentrated in vacuo and chromatographed on silica gel eluting with EtOAc:hexanes (1:9) to afford the title compound. δ_{H} (CDCl₃): 4.56 (2H, s), 7.50 (1H, m), 7.55 (1H, m), 7.63 (1H, m), 7.88 (1H, m), 7.92 (1H, m), 8.01 (1H, m), 8.63 (1H, m).

### Preparation 2

### 1-Naphthalen-2-ylpropan-1-one

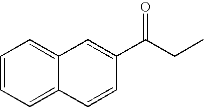

2-Naphthonitrile (3 g, 19.6 mmol) was dissolved in Et₂O (20 mL) followed by addition of ethylmagnesium chloride (2.0M in Et₂O, 9.8 mL, 19.6 mmol) and the reaction was heated to reflux for 5 hr, then stirred at rt for 16 hr. The reaction was quenched with 2N HCl (20 mL) and water (20 mL) then extracted into DCM (3×40 mL). The combined organic fractions were dried (MgSO₄), concentrated in vacuo and purified by chromatography on silica gel eluting with EtOAc:hexanes (1:9) to afford the title compound. δ_{H} (CDCl₃): 1.30 (3H, t), 3.13 (2H, q), 7.57 (2H, m), 7.88 (2H, m), 7.96 (1H, d), 8.05 (1H, dd), 8.47 (1H, s).

### Preparation 3

### 2-Bromo-1-naphthalen-2-ylpropan-1-one

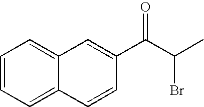

1-Naphthalen-2-ylpropan-1-one (Preparation 2, 3.5 g, 19.0 mmol) was dissolved in THF (50 mL) followed by addition of PTAT (7.1 g, 19.0 mmol) and the reaction stirred at rt for 16 hr. The solid was filtered and the filtrate concentrated in vacuo and purified on silica gel eluting with EtOAc:hexanes (1:9) to afford the title compound. δ_{H} (CDCl₃): 1.98 (3H, d), 5.47 (1H, q), 7.57 (1H, m), 7.63 (1H, m), 7.91 (2H, m), 7.99 (1H, d), 8.08 (1H, dd), 8.57 (1H, s).

### Preparation 4

### Thieno[2,3-b]thiophene-2-carboxylic acid methoxymethylamide

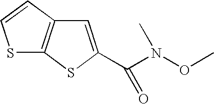

Thieno[2,3-b]thiophene-2-carboxylic acid (1 g, 5.43 mmol), N,O-dimethylhydroxylamine hydrochloride (0.53 g, 5.43 mmol) and HOBt (0.73 g, 5.43 mmol) were dissolved in DMF (20 mL) and DIPEA (2.9 mL, 16.8 mmol). After 5 min, EDCI (1.35 g, 7.1 mmol) was added and the reaction stirred at rt for 24 hr. The solvent was removed in vacuo and the residue partitioned between water (30 mL) and EtOAc (3×30 mL). The combined organic fractions were washed with 1N NaOH (2×20 mL), 1N HCl (2×20 mL), brine (20 mL), dried (MgSO₄) and concentrated in vacuo. The residue was purified by chromatography on silica gel eluting with EtOAc:hexanes (2:3) to afford the title compound. δ_{H} (CDCl₃): 3.40 (3H, s), 3.82 (3H, s), 7.28 (1H, d), 7.39 (1H, d), 8.13 (1H, s).

### Preparation 5

### 1-Thieno[2,3-b]thiophen-2-ylethanone

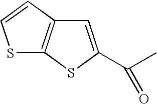

Thieno[2,3-b]thiophene-2-carboxylic acid methoxymethylamide (Preparation 4, 0.958 g, 4.21 mmol) was dissolved in THF (20 mL) under an argon atmosphere and cooled to 0° C. Methylmagnesium bromide (1.4M in toluene:THF, 6.3 mL, 8.85 mmol) was added dropwise and the reaction stirred at 0° C. for 2 hr, then rt for 16 hr. The reaction was quenched with 5% HCl in MeOH then the solvent removed in vacuo. The residue was partitioned between 10% NaHCO₃ solution (50 mL) and EtOAc (3×40 mL). The combined organic fractions were dried (MgSO₄), concentrated in vacuo and chromatographed on silica gel eluting with EtOAc:hexanes (1:3) to afford the title compound. δ_{H} (CDCl₃): 2.60 (3H, s), 7.28 (1H, d), 7.41 (1H, d), 7.83 (1H, s).

### Preparation 6

### 2-Bromo-1-thieno[2,3-b]thiophen-2-ylethanone

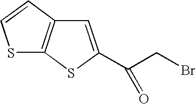

1-Thieno[2,3-b]thiophen-2-ylethanone (Preparation 5, 400 mg, 2.2 mmol) was dissolved in TI (20 mL) followed by addition of PTAT (825 mg, 2.2 mmol). The reaction was stirred at rt for 16 hr and the precipitated solid was filtered and washed with THF (2×20 mL). The filtrate was concentrated in vacuo and the residue partitioned between water (30 mL) and EtOAc (3×30 mL). The combined organic fractions were dried (MgSO₄), concentrated in vacuo and chromatographed on silica gel eluting with EtOAc:hexanes (1:4) to afford the title compound. δ_{H} (CDCl₃): 4.39 (2H, s), 7.32 (1H, d), 7.45 (1H, d), 7.96 (1H, s).

### Preparation 7

### 2-Bromo-1-(4-methylnaphthalen-1-yl)ethanone

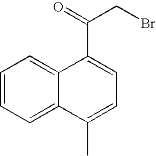

A mixture of 4-methyl-1-acetophone (0.5 g, 2.7 mmol) and PTAT (1.1 g, 3.0 mmol) in anhydrous THF (20 mL) was stirred at rt under argon for 3 hr. The reaction mixture was filtered and washed several times with THF. The filtrate was concentrated in vacuo and the residue partitioned between water (30 mL) and EtOAc (3×30 mL). The combined organic phase was dried (MgSO₄), concentrated in vacuo, and purified by chromatography on silica gel eluting with EtOAc:hexane (1:10) to afford the title compound. δ_{H} (CDCl₃): 2.77 (3H, s) 4.57 (2H, s), 7.37 (1H, d), 7.63 (2H, m), 7.86 (1H, d), 8.07 (1H, d), 8.71 (1H, d).

### Preparation 8

### Quinoline-2-carboxylic acid methoxymethylamide

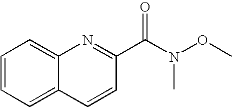

To a suspension of quinoline-2-carboxylic acid (2.5 g, 14.4 mmol), O,N-dimethylhydroxylamine hydrochloride (2.9 g, 29.7 mmol), EDCI (3.4 g, 17.7 mmol) and HOBt monohydrate (2.25 g, 14.7 mmol) in DMF (55 mL) was added DIPEA (10.5 mL, 61.3 mmol). The resulting solution was stirred for 12 hr at rt before the reaction mixture was partitioned between EtOAc (100 mL) and water:brine (200 mL, 1:1). The layers were separated and the aqueous phase was extracted with EtOAc (3×50 mL). After washing with dilute NaOH solution (1M, 50 mL) and brine (50 mL) the combined organic extracts were dried (MgSO₄), filtered and concentrated in vacuo. Purification of the residue by flash-chromatography on silica gel (eluent: EtOAc) gave the title compound. δ_{H} (DMSO): 3.34, 3.36 (6H, 2s), 7.71 (2H, m), 7.86 (1H, m), 8.08 (2H, m), 8.52 (1H, d); m/z (ES⁺)=217.09 [M+H]⁺; RT=2.79 min.

### Preparation 9

### 1-Quinolin-2-ylethanone

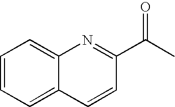

Method A: Methylmagnesium bromide (1.4M solution in toluene:THF, 3:1, 8 mL) was added to a solution of quinoline-2-carboxylic acid methoxymethyl amide (Preparation 8, 2.0 g, 9.25 mmol) in THF at 0° C. under an atmosphere of argon. After stirring in the cold for 2 hr the reaction mixture was added to conc. NH₄Cl solution (400 mL). Extraction with EtOAc (4×100 mL) followed by washing of the combined extracts with brine (150 mL), drying (MgSO₄), concentration in vacuo and purification of the residue by flash-chromatography on silica gel (eluent, hexane:EtOAc, 2:1) gave the title compound.

Method B: Methyl lithium (1.6M solution in diethyl ether, 10 mL) was added to a solution of quinoline-2-carboxylic acid (1.40 g, 8.1 mmol) in THF (40 mL) at 0° C. under an atmosphere of argon. After 2 hr successively chlorotrimethylsilane (10 mL, 79 mmol) and then after a period of 10 min dilute hydrochloric acid (1M, 30 mL) were added under vigorous stirring. The aqueous layer was separated, further diluted with water (200 mL) and neutralised with solid NaHCO₃. Similar work-up and purification to Method A gave the title compound. δ_{H} (DMSO): 2.80 (3H, s), 7.78 (1H, m), 7.90 (1H, m), 8.07 (1H, d), 8.81 (1H, d), 8.20 (1H, d), 8.57 (1H, d); m/z (ES⁺)=172.10 [M+H]⁺; RT=3.34 min.

### Preparation 10

### 2-[2-Bromo-(isopropyldimethylsilanyloxy)vinyl]quinoline

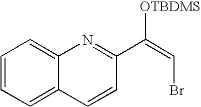

Triethylamine (0.40 mL, 2.85 mmol) and tert-butyldimethylsilyl chloride (0.35 mL, 1.52 mmol) were added to a solution of 1-quinolin-2-ylethanone (Preparation 9, 240 mg, 1.40 mol) in dry DCM (10 mL) at 0° C. under an atmosphere of argon. After 1 hr PTAT (535 mg, 1.42 mmol) was added, the ice bath was removed and the resulting mixture was stirred for an additional 2 hr. Partitioning between EtOAc (100 mL) and a mixture of dilute Na₂S₂O₃ solution (10%, 50 mL) and NaHCO₃ solution (50 mL) was followed by further extraction of the aqueous phase with EtOAc (3×50 mL). After washing with brine (50 mL) the combined organic extracts were dried (MgSO₄), filtered and concentrated in vacuo. Purification of the residue by flash-chromatography on silica gel (eluent, hexane:EtOAc, 4:1) gave the title compound. δ_{H} (DMSO): 0.19 (6H, s), 1.01 (9H, s), 6.91 (1H, s), 7.42 (1H, m), 7.60 (1H, m), 7.63 (1H, d), 7.78 (1H, d), 7.80 (1H, d), 8.38 (1H, d); m/z (ES⁺)=364.09, 364.09 [M+H]⁺; RT=4.95 min.

### Preparation 11

### 2-Bromo-1-quinolin-2-ylethanone

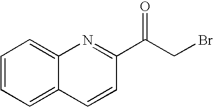

Method A: A suspension of pyridinium tribromide (420 mg, 1.31 mmol) in AcOH (10 mL) was treated with hydrobromic acid (30% in AcOH, 0.26 mL) and then stirred for 30 min at rt A solution of 1-quinolin-2-yl-ethanone (Preparation 9, 200 mg, 1.08 mmol) was added and the mixture was stirred for 12 hr at rt. After removal of the solvent the residue was partitioned between EtOAc (100 mL) and conc. NaHCO₃ solution (100 mL). The layers were separated and the aqueous phase was extracted with EtOAc (3×50 mL). Washing of the combined extracts with brine (100 mL), drying (MgSO₄) and concentration in vacuo followed by recrystallisation from hexane gave the title compound.

Method B: Hydrobromic acid (30% in AcOH, 0.46 mL) was added to a solution of 2-[2-bromo-1-isopropyldimethylsilanyloxy)vinyl]quinoline (Preparation 10, 420 mg, 1.15 mmol) in THF (20 mL) at −78° C. under an atmosphere of argon. The IPA/dry-ice bath was removed and the mixture was stirred for 12 hr at rt. Similar work-up and purification to Method A gave the title compound. δ_{H} (DMSO): 5.24 (2H, s), 7.82 (1H, m), 7.94 (1H, m), 8.14 (2H, m), 8.21 (1H, d), 8.63 (1H, d); m/z (ES⁺)=249.98, 251.98 [M+H]⁺; RT=3.65 min.

### Preparation 12

### 2-Bromo-1-(4-fluoronaphthalen-1-yl)ethanone

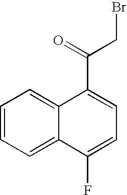

4-Fluoro-1-acetonaphthone (500 mg, 2.66 mmol) was dissolved in THF (20 mL) followed by addition of PTAT (1.1 g, 2.92 mmol). The reaction was stirred at rt for 16 hr then the precipitated solid was filtered and washed with THF (2×20 mL). The filtrate was concentrated in vacuo and purified by chromatography on silica gel eluting with EtOAc:hexanes (1:9) to afford the title compound. δ_{H} (CDCl₃): 4.55 (2H, s), 7.19 (1H, m), 7.64 (1H, m), 7.72 (1H, m), 7.99 (1H, m), 8.19 (1H, d), 8.79 (1H, d).

### Preparation 13

### 3-Naphthalen-2-yl-3-oxopropionic acid ethyl ester

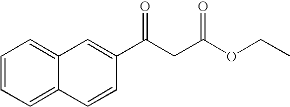

Methyl β-naphthylketone (5 g, 29.4 mmol) was dissolved in diethyl carbonate (50 mL) under an argon atmosphere and sodium hydride (60% in mineral oil, 2.35 g, 58.8 mmol) was added portionwise over 10 min. The reaction mixture was heated to 100° C. for 4 hr then stirred at rt for 16 hr. The solvent was removed in vacuo and the residue partitioned between AcOH (5 mL) in water (200 mL) and Et₂O (3×100 mL). The combined organic fractions were washed with brine (50 mL), dried (MgSO₄) and concentrated in vacuo. The residue was purified by chromatography on silica gel eluting with EtOAc:hexanes (1:9 to 1:4) to afford the title compound. δ_{H} (CDCl₃): 1.27 (3H, t), 4.12 (2H, s), 4.24 (2H, q), 7.58 (2H, m), 7.88 (2H, m), 7.96 (1H, d), 8.02 (1H, m), 8.45 (1H, s).

### Preparation 14

### 2-Bromo-3-naphthalen-2-yl-3-oxopropionic acid ethyl ester

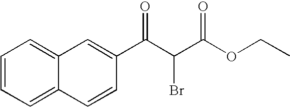

3-Naphthalen-2-yl-3-oxopropionic acid ethyl ester (Preparation 13, 6.33 g, 26.1 mmol) was dissolved in THF (100 mL) and cooled to 0° C. PTAT (9.82 g, 26.1 mmol) was added and the reaction was stirred at 0° C. for 2 hr then at rt for 16 hr. The precipitated solid was filtered and washed with THF (2×30 mL). The filtrate was concentrated in vacuo and purified by chromatography on silica gel eluting with EtOAc:hexanes (1:9) to afford the title compound. δ_{H} (CDCl₃): 1.25 (3H, t), 4.30 (2H, q), 5.84 (1H, s), 7.58 (1H, m), 7.64 (1H, m), 7.90 (2H, m), 7.97 (1H, d), 8.02 (1H, dd), 8.53 (1H, s).

### Preparation 15

### 1-Naphthalen-1-ylpropanone

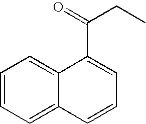

1-Cyanonaphthalene (4.6 g, 30 mmol) was dissolved in Et₂O (15 mL) followed by addition of ethylmagnesium chloride (2.0M in Et₂O, 15 mL) and the reaction heated to reflux for 17 hr. The reaction was the quenched with dilute HCl (2N, 20 mL) and the mixture heated for a further 1 hr before water (20 mL) was added and the mixture separated and extracted with DCM (3×40 mL). The combined organic fractions were dried (MgSO₄), concentrated in vacuo and purified by chromatography on silica gel eluting with Et₂O:iso-hexane (1:19) to afford the title compound. δ_{H} (CDCl₃): 1.27 (3H, t), 3.12 (2H, q), 7.58 (3H, m), 7.90 (2H, m), 8.02 (1H, d), 8.61 (1H, d); m/z (ES⁺)=184.09 [M+H]⁺; RT=2.37 min.

### Preparation 16

### 2-Bromo-1-naphthalen-1-ylpropanone

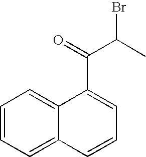

To a stirred solution of 1-naphthalen-1-ylpropanone (Preparation 15, 3.0 g, 16.3 mmol) in THF (20 mL) was added PTAT (6.3 g, 16.3 mmol) and the reaction was stirred at rt for 16 hr. The reaction mixture was filtered and the solid washed with THF (2×20 mL). The filtrate was concentrated in vacuo and the residue partitioned between EtOAc (50 mL) and saturated sodium bicarbonate solution (50 mL). The combined organics were dried (MgSO₄) and concentrated in vacuo to afford the title compound. δ_{H} (CDCl₃): 2.0 (3H, d), 5.4 (1H, q), 7.60 (3H, m), 7.90 (2H, m), 8.06 (1H, d), 8.49 (1H, d).

### Preparation 17

### 3-Naphthalen-1-yl-5,6-dihydroimidazo[2,1-b]thiazole-2-carbaldehyde

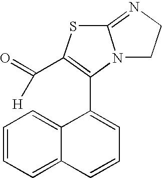

To a solution of ethylmagnesium chloride (2.0M in Et₂O, 3.6 mL, 7.28 mmol) in THF (20 mL) cooled to 0° C. was added 2-bromo-3-naphthalen-1-yl-5,6-dihydroimidazo[2,1-b]thiazole hydrobromide (Example 4, 1 g, 2.43 mmol) portionwise over 10 min, and the reaction was stirred at 0° C. for 2 hr. DMF (0.75 mL, 9.71 mmol) was added over 5 min and the reaction stirred at 0° C. for 30 min then rt for 16 hr. The reaction was quenched with saturated NH₄Cl solution (40 mL) and water (20 mL) then extracted into EtOAc (3×40 mL). The combined organic fractions were dried (MgSO₄), concentrated in vacuo and purified by chromatography on silica gel eluting with MeOH:DCM (1:19) to afford the title compound. δ_{H} (CDCl₃): 3.55 (2H, m), 4.31 (2H, t), 7.63 (4H, m), 7.79 (1H, m), 7.99 (1H, m), 8.07 (1H, m), 9.13 (1H, s).

### Preparation 18

### 1-(7-Chloronaphthalen-1-yl)propan-1-one

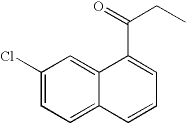

Aluminium chloride (12.5 g, 93.7 mmol) was added to a solution of 2-chloronaphthalene (5.0 g, 30.7 mmol) in DCM (50 mL) at −10° C. After stirring for 20 min the mixture was cooled to −78° C. and propionyl chloride (5.5 mL, 63.3 mmol) was added dropwise. The resulting suspension was maintained at −78° C. for 4 hr before being added to dilute HCl (0.3M, 300 mL). The layers were separated and the aqueous layer was further extracted with EtOAc (2×100 mL). The extracts were combined, washed with brine (100 mL) and dried (MgSO₄). Concentration followed by purification of the residue by flash chromatography on silica gel (eluent, hexane:EtOAc, 10:1) gave the title compound. δ_{H} (DMSO): 1.16 (3H, t), 3.15 (2H, q), 7.62-7.67 (2H, m), 8.07 (1H, d), 8.19-8.21 (2H, m), 8.62 (1H, s); m/z (ES⁺)=219.01 [M+H]⁺; RT=4.02 min.

### Preparation 19

### 2-Bromo-1-(7-chloronaphthalen-1-yl)propan-1-one

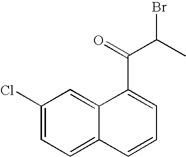

PTAT (2.21 g, 5.88 mmol) was added to a solution of 1-(7-chloronaphthalen-1-yl)propan-1-one (Preparation 18, 1.28 g, 5.85 mmol) in THF (50 mL). Whilst stirring at rt for 2 hr the bright orange solution was almost completely decolorized and a precipitate was formed. The mixture was filtered through Celite and the filtrate diluted with EtOAc (300 mL) and washed with dilute Na₂S₂O₃ solution (10%, 100 mL) and brine (100 mL). Drying (MgSO₄) and concentration in vacuo, followed by recrystallisation from EtOH gave the title compound. δ_{H} (DMSO): 1.98 (3H, d), 5.94 (1H, q), 7.67-7.71 (2H, m), 8.12 (1H, d), 8.26 (1H, d), 8.31 (1H, d), 8.42 (1H, s); m/z (ES⁺)=298.94 [M+H]⁺; RT=4.12 min.

### Preparation 20

### 3-Naphthalen-2-yl-5,6-dihydroimidazo[2,1-b]thiazole-2-carbaldehyde

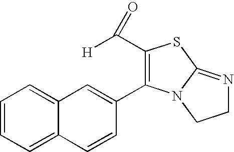

2-Bromo-3-naphthalen-2-yl-5,6-dihydroimidazo[2,1-b]thiazole hydrobromide (Example 11, 4.0 g, 9.7 mmol) was suspended in THF (50 mL) under an argon atmosphere and cooled to 0° C. Ethylmagnesium chloride (2.0M in Et₂O, 14.6 mL, 29.1 mmol) was added and the reaction stirred at 0° C. for 1 hr then rt for 1 hr. The reaction was cooled to 0° C. and DMF (3.0 mL, 38.8 mmol) was added. The reaction was stirred at 0° C. for 1 hr then rt for 16 hr. The reaction was quenched with saturated NH₄Cl solution (50 mL) and water (25 mL) then extracted into EtOAc (3×50 mL). The combined organic fractions were dried (MgSO₄), concentrated in vacuo and purified by chromatography on silica gel eluting with MeOH:DCM (1:19) to afford the title compound. δ_{H} (CDCl₃): 3.87 (2H, m), 4.35 (2H, m), 7.54 (1H, dd), 7.64 (2H, m), 7.94 (2H, m), 8.02 (2H, m), 9.36 (1H, s).

### Preparation 21

### 1-(4-Fluoronaphthalen-1-yl)propan-1-one

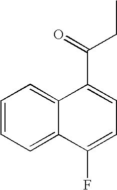

1-Cyano-4-fluoronaphthalene (1 g, 5.8 mmol) was dissolved in Et₂O (10 mL) followed by addition of ethylmagnesium chloride (2.0M in Et₂O, 2.9 mL, 5.8 mmol) and the reaction was heated to reflux for 16 hr. The reaction was quenched with 2N HCl (20 mL) then extracted into DCM (3×40 mL). The combined organic fractions were dried (MgSO₄), concentrated in vacuo and purified by chromatography on silica gel eluting with EtOAc:hexanes (1:9) to afford the title compound. δ_{H} (CDCl₃): 1.29 (3H, t), 3.08 (2H, q), 7.16 (1H, m), 7.60 (1H, m), 7.66 (1H, m), 7.89 (1H, m), 8.16 (1H, d), 8.74 (1H, d).

### Preparation 22

### 2-Bromo-1-(4-fluoronaphthalen-1-yl)propan-1-one

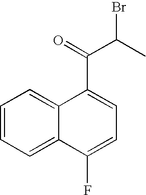

1-(4-Fluoronaphthalen-1-yl)propan-1-one (Preparation 21, 3.30 g, 16.3 mmol) was dissolved in THF (50 mL) and cooled to 0° C. PTAT (6.14 g, 16.3 mmol) was added and the reaction mixture stirred at rt for 16 hr. The solid was filtered and washed with THF (2×20 mL). The filtrate was concentrated in vacuo and the residue partitioned between water (30 mL) and EtOAc (2×50 mL). The combined organic fractions were washed with brine (3×20 mL) dried (MgSO₄), concentrated in vacuo and purified by chromatography on silica gel eluting with DCM to afford the title compound. δ_{H} (CDCl₃): 1.98 (3H, d), 5.36 (1H, q), 7.18 (1H, m), 7.63 (1H, m), 7.70 (1H, m), 7.92 (1H, m), 8.18 (1H, d), 8.59 (1H, d).

### Preparation 23

### 1-Naphthalen-1-ylbutan-1-one

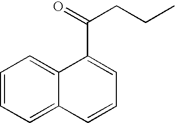

1-Cyanonaphthalene (1 g, 6.5 mmol) was dissolved in THF (20 mL) under an argon atmosphere and cooled to 0° C. Ethylmagnesium chloride (2.0M in Et₂O, 9.8 mL, 19.6 mmol) was added and the reaction stirred at 0° C. for 1 hr then rt for 16 hr. The reaction was quenched with 1N HCl (40 mL) and extracted into DCM (3×30 mL). The combined organic fractions were dried (MgSO₄), concentrated in vacuo and purified on silica gel eluting with EtOAc:hexanes (1:4) to afford the title compound. δ_{H} (CDCl₃): 1.05 (3H, t), 1.84 (2H, m), 3.04 (2H, t), 7.48-7.71 (3H, m), 7.87 (2H, m), 7.98 (1H, d), 8.57 (1H, d).

### Preparation 24

### 2-Bromo-1-naphthalen-1-ylbutan-1-one

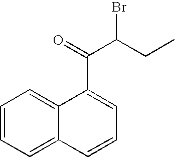

1-Naphthalen-1-ylbutan-1-one (Preparation 23, 0.99 g, 5.0 mmol) was dissolved in THF (20 mL) and cooled to 0° C. PTAT (1.88 g, 5.0 mmol) was added and the reaction stirred at rt for 16 hr. The precipitated solid was filtered and washed with THF (2×10 mL). The filtrate was concentrated in vacuo and the residue partitioned between water (40 mL) and EtOAc (2×40 mL). The combined organic fractions were washed with brine (20 mL), dried (MgSO₄), concentrated in vacuo and purified by chromatography on silica gel eluting with EtOAc:hexanes (1:9) to afford the title compound. δ_{H} (CDCl₃): 1.15 (3H, t), 2.20 (1H, m), 2.33 (1H, m), 5.16 (1H, t), 7.50-7.65 (3H, m), 7.89 (2H, m), 8.03 (1H, d), 8.47 (1H, d).

### Preparation 25

### 3-Methyl-1-naphthalen-1-ylbutan-1-one

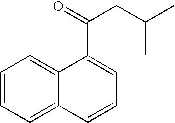

1-Cyanonaphthalene (1 g, 6.5 mmol) was dissolved in THF (20 mL) under an argon atmosphere and cooled to 0° C. Isobutylmagnesium bromide (2.0M in Et₂O, 9.8 mL, 19.6 mmol) was added and the reaction stirred at 0° C. for 1 hr then rt for 16 hr. The reaction was quenched with 1N HCl (30 mL) then extracted into DCM (3×30 mL). The combined organic fractions were dried (MgSO₄), concentrated in vacuo and purified by chromatography on silica gel eluting with EtOAc:hexanes (1:9) to afford the title compound. δ_{H} (CDCl₃): 1.04 (6H, d), 2.35 (1H, m), 2.95 (2H, d), 7.48-7.62 (3H, m), 7.83 (1H, d), 7.89 (1H, d), 7.98 (1H, d), 8.56 (1H, d).

### Preparation 26

### 2-Bromo-3-methyl-1-naphthalen-1-ylbutan-1-one

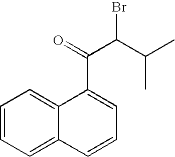

3-Methyl-1-naphthalen-1-ylbutan-1-one (Preparation 25, 0.36 g, 1.7 mmol) was dissolved in THF (10 mL) and cooled to 0° C. PTAT (0.64 g, 1.7 mmol) was added and the reaction stirred at rt for 72 hr. The solid was filtered and washed with THF (2×20 mL). The filtrate was concentrated in vacuo and the residue partitioned between water (40 mL) and EtOAc (2×40 mL). The combined organic fractions were washed with brine (2×20 mL), dried MgSO₄), concentrated in vacuo and purified by chromatography on silica gel eluting with EtOAc:hexanes (1:9) to afford the title compound. δ_{H} (CDCl₃): 1.13 (3H, d), 1.24 (3H, d), 2.53 (1H, m), 5.03 (1H, d), 7.52 (1H, m), 7.57 (1H, m), 7.63 (1H, m), 7.89 (2H, m), 8.03 (1H, d), 8.46 (1H, d).

### Preparation 27

### 5-Chloronaphthalene-1-carboxylic acid methoxymethylamide

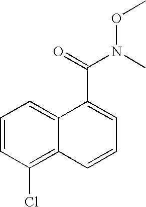

To a solution of 5-chloro-1-naphthoic acid (1.12 g, 5.4 mmol) in DMF (10 mL), N,O-dimethylhydroxylamine (0.53 g, 5.4 mmol), HOBt (0.73 g, 5.4 mmol) and DIPEA (2.9 mL, 17.0 mmol) were added. After 5 min EDCI (1.35 g, 7.0 mmol) was added and the reaction mixture stirred at rt under argon for 96 hr. The solvent was removed in vacuo and the oil partitioned between water (100 mL) and EtOAc (3×100 mL). The combined organic phase was dried (MgSO₄), concentrated in vacuo and purified by chromatography on silica gel eluting with EtOAc:hexane (1:1) to afford the title compound. δ_{H} (CD₃OD): 3.42 (6H, br s), 7.51 (1H, m), 7.62 (1H, d), 7.68 (2H, m), 7.79 (1H, d), 8.37 (1H, d).

### Preparation 28

### 1-(5-Chloronaphthalen-1-yl)propan-1-one

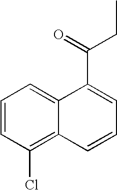

A stirred solution of 5-chloronaphthalene-1-carboxylic acid methoxymethylamide (Preparation 27, 0.89 g, 3.6 mmol) and THF (50 mL) was cooled to 0° C. under an argon atmosphere. Ethylmagnesium bromide (2.0M in Et₂₀, 1.2 mL, 3.6 mmol) was added dropwise and stirred at rt for 24 hr. Further ethylmagnesium bromide (1.2 mL, 3.6 mmol) was added and the reaction stirred at rt for a further 24 hr. The reaction was acidified with conc. HCl (5 mL) in methanol (40 mL) and concentrated in vacuo. The residue was partitioned between water (100 mL) and EtOAc (3×100 mL). The combined organic phase was dried (MgSO₄), concentrated in vacuo and purified by chromatography on silica gel eluting with EtOAc:hexane (1:9) to give the title compound. δ_{H} (CDCl₃): 1.29 (3H, t), 3.08 (2H, q), 7.48 (1H, m), 7.63 (2H, m), 7.88 (1H, d), 8.44 (1H, d), 8.48 (1H, d).

### Preparation 29

### 2-Bromo-1-(5-chloronaphthalen-1-yl)propan-1-one

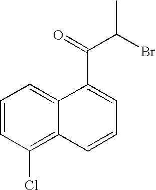

A mixture of 1-(5-chloronaphthalen-1-yl)propan-1-one Preparation 28, 0.13 g, 0.6 mmol) and PTAT (0.25 g, 0.66 mmol) in anhydrous THF (10 mL) was stirred at rt under argon for 48 hr. The reaction mixture was filtered and washed several times with THF. The filtrate was concentrated in vacuo giving an oil which was partitioned between water (100 mL) and EtOAc (3×100 mL). The combined organic phase was dried (MgSO₄), concentrated in vacuo and purified by chromatography on silica gel eluting with EtOAc:hexane (1:19) to give the title compound. δ_{H} (CDCl₃): 1.99 (3H, d), 5.35 (1H, q), 7.53 (1H, m), 7.65 (2H, m), 7.89 (1H, d), 8.31 (1H, d), 8.53 (1H, d).

### Preparation 30

### 6-Fluoronaphthalene-2-carboxylic acid methoxymethylamide

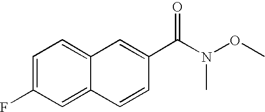

To a solution of 6-fluoro-2-naphthoic acid (1.13 g, 5.9 mmol) in DMF (10 mL) was added N,O-dimethylhydroxylamine (0.58 g 5.9 mmol), HOBt (0.80 g, 5.9 mmol) and DIPEA (3.2 mL, 18.2 mmol). After 5 min, EDCI (1.47 g, 7.7 mmol) was added and the reaction mixture stirred at rt under argon for 24 hr. The solvent was removed in vacuo and the residue partitioned between EtOAc (3×50 mL) and water (50 mL). The combined organic phase was dried (MgSO₄), concentrated in vacuo and purified by chromatography on silica gel eluting with EtOAc:hexane (1:1) to give the title compound. δ_{H} (CDCl₃): 3.42 (3H, s), 3.57 (3H, s), 7.31 (1H, m), 7.48 (1H, dd), 7.80 (2H, s), 7.91 (1H, m), 8.24 (1H, s).

### Preparation 31

### 1-(6-Fluoronaphthalen-2-yl)propan-1-one

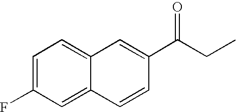

Ethylmagnesium chloride (2.0M in Et₂O, 5.5 mL, 11 mmol) was added dropwise to a solution of 6-fluoronaphthalene-2-carboxylic acid methoxymethylamide (Preparation 30, 0.18 g, 5.0 mmol) in THF (50 mL) at 0° C. under an argon atmosphere. The reaction mixture was warmed to rt and stirred for 24 hr. The reaction was acidified with conc. HCl (5 mL) in methanol (40 mL) and concentrated in vacuo the resulting solid was partitioned between EtOAc (3×100 mL) and water (100 mL). The combined organic phase was dried (MgSO₄), concentrated in vacuo affording the title compound. δ_{H} (CDCl₃): 1.30 (3H, t), 3.14 (2H, q), 7.33 (1H, td), 7.50 (1H, dd), 7.84 (1H, d), 7.97 (1H, m), 8.08 (1H, d), 8.48 (1H, s).

### Preparation 32

### 2-Bromo-1-(6-fluoronaphthalen-2-yl)propan-1-one

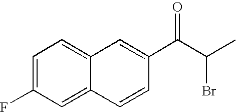

A mixture of 1-(6-fluoronaphthalen-2-yl)propan-1-one (Preparation 31, 0.84 g, 4.0 mmol) and PTAT (1.72 g, 4.6 mmol) in anhydrous THF (10 mL) was left to stir at rt for 24 hr. The reaction mixture was filtered and washed several times with THF. The filtrate was concentrated in vacuo to afford the title compound. δ_{H} (CDCl₃): 1.98 (3H, d), 5.44 (1H, q), 7.36 (1H, td), 7.51 (1H, dd), 7.88 (1H, d), 8.00 (1H, m), 8.10 (1H, d), 8.57 (1H, s).

### Preparation 33

### 6-Chloronaphthalene-2-carboxylic acid

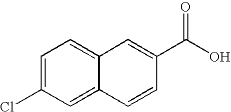

A stirred solution of 6-amino-2-naphthoic acid (3.14 g, 17.0 mmol) in water (10 mL) and conc. HCl (20 mL) was cooled to 0° C. and a solution of sodium nitrite (1.17 g. 17.0 mmol) in water (10 mL) was added dropwise. After 1 hr a solution of copper(I)chloride (3.36 g, 34.0 mmol) in conc. HCl (10 mL) at 0° C. was added portionwise. The reaction mixture was diluted with water (400 mL) and stirred for 30 min. The precipitated solid was collected by filtration, washed several times with water and dried to afford the title compound. δ_{H} (DMSO): 7.63 (1H, dd), 8.03 (2H, m), 8.17 (2H, m), 8.64 (1H, s).

### Preparation 34

### 6-Chloronaphthalene-2-carboxylic acid methoxymethylamide

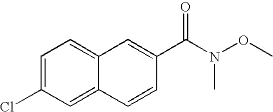

To a solution of 6-chloronaphthalene-2-carboxylic acid (Preparation 33, 1.65 g, 8.0 mmol) in DMF (10 mL) was added N,O-dimethylhydroxylamine (0.78 g 8 mmol), HOBt (1.08 g, 8 mmol) and DIPEA (4.3 mL, 25 mmol). After 5 min EDCI (2.0 g, 10.0 mmol) was added and the reaction mixture stirred at rt under argon for 24 hr. The solvent was removed in vacuo and the residue partitioned between EtOAc (3×100 mL) and water (100 mL). The combined organic phase was dried (MgSO₄) and concentrated in vacuo. The residue was dissolved in EtOAc (100 mL) and washed with HCl (2M, 50 mL). The organic layer was washed with sodium hydroxide (2M, 50 mL), dried (MgSO₄) and concentrated in vacuo to afford the title compound. δ_{H} (CDCl₃): 3.42 (3H, s), 3.56 (3H, s), 7.48 (1H, dd), 7.83, (4H, m), 8.21 (1H, s).

### Preparation 35

### 1-(6-chloronaphthalen-2-yl)propan-1-one

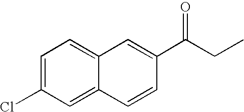

A stirred solution of 6-chloronaphthalene-2-carboxylic acid methoxymethylamide (Preparation 34, 1.84 g, 7.4 mmol) and THF (50 mL) was cooled to 0° C. under an argon atmosphere. Ethylmagnesium chloride (2M in Et₂O, 7.7 mL, 15.4 mmol) was added dropwise and stirred at rt for 24 hr. The reaction mixture was acidified with conc. HCl (5 mL) in methanol (40 mL) and concentrated in vacuo. The residue was partitioned between EtOAc (3×100 mL) and sodium bicarbonate (100 mL). The combined organic phase was dried (MgSO₄) and concentrated in vacuo to afford the title compound. δ_{H} (CDCl₃): 1.30 (3H, t), 3.14 (2H, q), 7.51 (1H, dd), 7.82 (1H, d), 7.90 (2H, m), 8.08 (1H, dd), 8.46 (1H, s).

### Preparation 36

### 2-Bromo-1-(6-chloronaphthalen-2-yl)propan-1-one

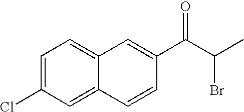

A mixture of 1-(6-chloronaphthalen-2-yl)propan-1-one (Preparation 35, 1.39 g, 6.4 mmol) and PTAT (2.64 g, 7.0 mmol) in anhydrous THF (40 mL) was stirred at rt under argon for 24 hr. The reaction mixture was filtered and the solid washed several times with THF. The filtrate was then concentrated in vacuo. The residue was purified by chromatography on silica gel eluting with EtOAc:hexane (8:92) to afford the title compound. δ_{H} (CDCl₃): 1.98 (3H, d), 5.43 (1H, q), 7.53 (1H, dd), 7.89 (3H, m), 8.11 (1H, dd), 8.55 (1H, s).

### Preparation 37

### 1-Naphthalen-2-ylbutan-1-one

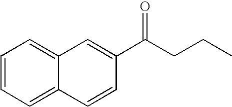

Propylmagnesium chloride (2.0M in Et₂O, 3.3 mL, 6.5 mmol) was added dropwise to a stirred solution of 2-naphthonitrile (1.0 g, 6.5 mmol) in Et₂O (20 mL) at 0° C. under an argon atmosphere. The reaction was warmed to rt and stirred for 48 hr. The reaction was acidified with HCl (2M, 15 mL) and concentrated in vacuo to afford the title compound. δ_{H} (CDCl₃): 1.06 (3H, t), 1.85 (2H, m), 3.09 (2H, t), 7.61 (2H, m), 7.90 (2H, m), 7.98 (1H, d), 8.05 (1H, d), 8.48 (1H, s).

### Preparation 38

### 2-Bromo-1-naphthalen-2-ylbutan-1-one

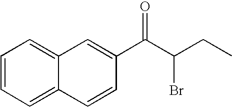

A mixture of 1-naphthalen-2-ylbutan-1-one (Preparation 37, 1.26 g, 6.4 mmol) and PTAT (2.65 g, 7.0 mmol) in anhydrous THF (20 mL) was stirred at rt under argon for 48 hr. The reaction mixture was filtered and the solid washed several times with THF. The filtrate was concentrated in vacuo giving a solid which was purified by chromatography on silica gel eluting with EtOAc:hexane (1:19) to afford the title compound. δ_{H} (CDCl₃): 1.14 (3H, t), 2.23 (1H, m), 2.30 (1H, m), 5.25 (1H, t), 7.61 (2H, m), 7.92 (2H, m), 8.00 (1H, d), 8.08 (1H, dd), 8.56 (1H, s).

### Preparation 39

### Benzo[b]thiophene-2,3-dione

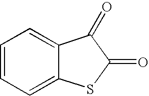

Oxalyl chloride (3.8 mL, 43.0 mmol) was added dropwise to a stirred solution of benzenethiol (3 g, 27.0 mmol) in Et₂O (20 mL) and the mixture heated under reflux for 1.5 hr. The solution was cooled to rt then concentrated in vacuo. The residue was dissolved in DCM (50 mL) and cooled to 0° C. Aluminium chloride (2.3 g, 32.0 mmol) was added portionwise to the reaction mixture and the solution heated under reflux for 1 hr. The solution was cooled to rt and poured onto ice. The organic layer was separated and washed successively with saturated aqueous sodium bicarbonate (100 mL), water (100 mL) and brine (100 mL). The organic layer was dried (MgSO₄) and concentrated in vacuo. With the addition of hexane a precipitate was formed which was collected by filtration to afford the title compound. δ_{H} (CDCl₃): 7.38 (1H, t), 7.43 (1H, d), 7.70 (1H, t), 7.84 (1H, d).

### Preparation 40

### Benzo[d]isothiazole-3-carboxylic acid amide

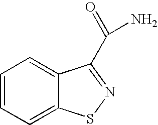

To a stirred solution of benzo[b]thiophene-2,3-dione (Preparation 39, 0.73 g, 4.4 mmol) in ammonium hydroxide (28% in H₂O, 14 mL) was added hydrogen peroxide (30% volume, 1.4 mL) dropwise over 5 min and stirred for 96 hr. The precipitate was collected by filtration to afford the title compound. δ_{H} (CDCl₃): 5.62 (1H, br s) 7.27 (1H, br s), 7.57 (2H, m), 7.97 (1H, d), 8.98 (1H, d).

### Preparation 41

### Benzo[d]isothiazole-3-carbonitrile

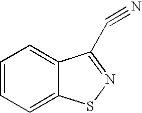

Benzo[d]isothiazole-3-carboxylic acid amide (Preparation 40, 0.52 g, 3.0 mmol) was dissolved in phosphorus oxylchloride (10 mL) at 0° C., warmed to rt then heated under reflux for 3 hr. The solvent was removed in vacuo and the residue partitioned between ice water (100 mL) and EtOAc (3×100 mL). The combined organic phase was dried (MgSO₄) and concentrated in vacuo to afford the title compound. δ_{H} (CDCl₃): 7.67 (2H, m), 8.06 (1H, d), 8.26 (1H, d).

### Preparation 42

### 1-Benzo[d]isothiazol-3-ylpropan-1-one

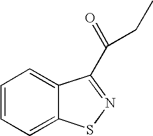

Ethylmagnesium bromide (3.0M in Et₂O, 0.83 mL, 2.5 mmol) was added dropwise to a stirred solution of benzo[d]isothiazole-3-carbonitrile (Preparation 41, 0.39 g, 2.5 mmol) in Et₂O (20 mL) at 0° C. and stirred for 24 hr under an argon atmosphere. The reaction was acidified with HCl (2M, 15 mL) and concentrated in vacuo the resulting solid was partitioned between water (100 mL) and EtOAc (3×100 mL). The combined organic phase was dried (MgSO₄) and concentrated in vacuo to afford the title compound. δ_{H} (CDCl₃): 1.29 (3H, t), 3.33 (2H, q), 7.56 (2H, m), 7.98 (1H, d), 8.88 (1H, d).

### Preparation 43

### 1-Benzo[d]isothiazol-3-yl-2-bromopropan-1-one

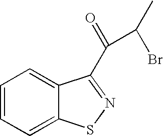

A mixture of 1-benzo[d]isothiazol-3-ylpropan-1-one (Preparation 42, 0.35 g, 1.8 mmol) and PTAT (0.74 g, 2.0 mmol) in anhydrous THF (10 mL) was stirred at rt under argon for 72 hr. The reaction mixture was filtered and washed several times with THF. The filtrate was concentrated in vacuo giving an oil which was partitioned between EtOAc (3×100 mL), water (100 mL) and brine (50 mL). The combined organic phase was dried (MgSO₄), concentrated in vacuo and purified by chromatography on silica gel eluting with EtOAc:hexane (1:19) to afford the title compound. δ_{H} (CDCl₃): 1.97 (3H, d), 5.99 (1H, q), 7.60 (2H, m), 8.00 (1H, dd), 8.85 (1H, dd); m/z (ES⁺)=274 [M+H]⁺; RT=2.12 min.

### Preparation 44

### 2-Bromo-1-(3,4-dichlorophenyl)propan-1-one

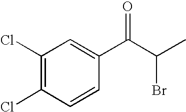

To a stirred solution of 1-(3,4-dichlorophenyl)propan-1-one (1.0 g, 4.924 mmol) in THF (30 mL) was added PTAT (1.94 g, 5.170 mmol) and the reaction was stirred at rt for 16 hr. The reaction mixture was filtered and the solid washed with THF (2×20 mL). The filtrate was concentrated in vacuo and the residue partitioned between a saturated solution of NaHCO₃ (30 mL) and DCM (2×30 mL). The combined organics were dried (MgSO₄), concentrated in vacuo and chromatographed on silica gel eluting with ethyl acetate:hexanes (1:4) to afford the title compound as a pale yellow oil. δ_{H} (CDCl₃): 8.20 (1H, s), 7.90 (1H, d), 7.60 (1H, d), 5.20 (1H, q), 1.95 (3H, m).

### Preparation 45

### 4-Bromo-N-methoxy-3,N-dimethylbenzamide

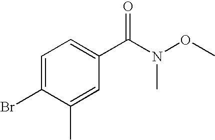

Diisopropylethylamine (7.3 mL, 0.042 mol) was added to a solution of 4-bromo-3-methylbenzoic acid (3 g, 0.014 mol), N,O-dimethylhydroxylamine hydrochloride (1.36 g, 0.014 mol), EDCI (4.01 g, 0.021 mol) and HOBt (1.94 g, 0.01395 mol) in DMF (20 ml) at rt. After 24 hr the solvent was removed in vacuo and the residue purified on silica by elution with DCM to yield the title compound. m/z (ES⁺)=257.93 [M+H]⁺; RT=3.31 min.

### Preparation 46

### 1-(4-Bromo-3-methylphenyl)ethanone

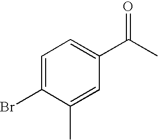

Methyllithium (3.7 mL, 5.93 mmol) was added to a solution of 4-bromo-N-methoxy-3,N-dimethylbenzamide (Preparation 45, 1.53 g, 5.93 mmol) in THF at −70° C. After warming to rt overnight the solvent was partially removed and the residue partitioned between dichloromethane (50 mL) and saturated ammonium chloride (50 mL). The organic layer was dried (MgSO₄), filtered and concentrated in vacuo. The residue was purified on silica gel by elution with hexane:DCM (4:1) to yield the title compound. δ_{H} (CDCl₃): 7.90 (1H, br s), 7.70 (2H, br s), 2.65 (3H, s), 2.45 (3H, s).

### Preparation 47

### 2-Bromo-1-(4-bromo-3-methylphenyl)ethanone

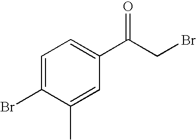

1-(4-Bromo-3-methylphenyl)ethanone (Preparation 46, 0.58 g, 2.732 mmol) and PTAT (1.027 g, 2.732 mmol) were added together in THF (30 ml) at rt. After overnight reaction the solid was filtered off and the filtrate concentrated to leave a brown oil. Purification on silica gel by elution with hexane:EtOAc (25:1) yielded the title compound. m/z (ES⁺)=292.97 [M+H]⁺; RT=2.60 min.

### Preparation 48

### 5-Chloronaphthalene-1-carboxylic acid methoxymethylamide

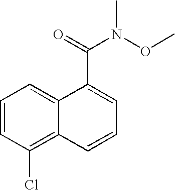

5-Chloro-1-naphthoic acid (1.12 g, 5.4 mmol), N,O-dimethylhydroxylamine hydrochloride (0.53 g, 5.4 mmol) and HOBt (0.73 g, 5.4 mmol) were dissolved in DMF (15 mL) and DIPEA (2.9 mL, 17.0 mmol). After 5 min, EDCI (1.35 g, 7 mmol) was added and the reaction was stirred at rt for 96 hr. The solvent was removed in vacuo and the residue partitioned between water (100 mL) and EtOAc (3×100 mL). The combined organic fractions were dried (MgSO₄), concentrated in vacuo and chromatographed on silica gel eluting with EtOAc:hexanes 1:1 affording the title compound. δ_{H} (CDCl₃): 3.41 (6H, br s), 7.44 (1H, t), 7.61 (3H, m), 7.83 (1H, d), 8.37 (1H, m).

### Preparation 49

### 1-(4-Chloronaphthalen-1-yl)propan-1-one

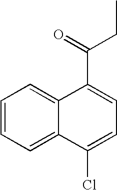

To a stirred solution of 1-chloronaphthalene (10 g, 61.5 mmol) in DCM (150 mL) at −10° C. was added aluminium chloride (24.6 g, 184.5 mmol) portionwise over 5 min, and the reaction mixture was cooled to −78° C. Propionyl chloride (10.7 mL, 123.0 mmol) was added dropwise over 10 min and the reaction stirred at −78° C. for 16 hr. The reaction mixture was warmed to 0° C. and quenched with 1M HCl, followed by addition of water (200 mL). The organic layer was separated and the aqueous layer extracted into DCM (2×125 mL). The combined organic fractions were washed with 1M NaOH solution (2×50 mL), brine (50 mL), dried (MgSO₄) and concentrated in vacuo affording the title compound. δ_{H} (CDCl₃): 1.28 (3H, t), 3.04 (2H, q), 7.58 (1H, d), 7.64 (2H, m), 7.73 (111, d), 8.34 (1H, m), 8.59 (1H, m).

### Preparation 50

### 5-Chloronaphthalene-1-carbonitrile

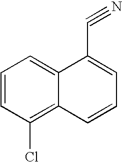

A solution of 5-chloro-1-naphthoic acid (2.08 g, 10.1 mmol) in thionyl chloride (10 mL) was refluxed for 3 hr, then cooled to rt and concentrated in vacuo. Concentrated ammonia solution (10 mL) was added cautiously to the residue at 0° C., and the reaction mixture was warmed to rt and stirred for 30 min. The reaction mixture was diluted with water (50 mL) and the solid was filtered, washed with water and dried under vacuum. The solid was dissolved in phosphorus oxychloride (10 mL) and heated to reflux for 4 hr, then cooled to rt and poured slowly into warm water (50 mL). The precipitated solid was filtered, washed with water (2×20 mL) and dried under vacuum affording the title compound. δ_{H} (CDCl₃): 7.64 (2H, m), 7.73 (1H, m), 8.00 (1H, m), 8.20 (1H, d), 8.56 (1H, d).

### Preparation 51

### Cyclopropylacetic acid methyl ester

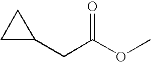

To a solution of cyclopropylacetic acid (2 g, 20.0 mmol) in MeOH (10 mL) was added concentrated H₂SO₄ (5 drops) and the reaction mixture was heated to reflux for 16 hr. The reaction mixture was cooled to rt and concentrated in vacuo. The residue was partitioned between saturated NaHCO₃ solution (30 mL) and Et₂O (2×30 mL). The combined organic fractions were washed with brine (2×20 mL), dried (MgSO₄) and concentrated in vacuo affording the title compound. δ_{H} (CDCl₃): 0.15 (2H, m), 0.54 (2H, m), 1.04 (1H, m), 2.21 (2H, d), 3.68 (3H, s).

### Preparation 52

### 2-Cyclopropyl-3-naphthalen-1-yl-3-oxopropionic acid methyl ester

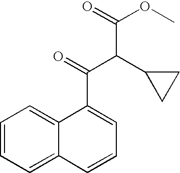

n-Butyllithium (2.5M, 8.3 mL, 20.9 mmol) was added to a solution of diisopropylamine (2.9 mL, 20.9 mmol) in THF (20 mL) at 0° C. under an argon atmosphere. The reaction mixture was stirred at 0° C. for 1 hr, and then cooled to −78° C. A solution of cyclopropylacetic acid methyl ester (Preparation 51, 1.19 g, 10.4 mmol) in THF (15 mL) was added dropwise and the reaction was stirred at −78° C. for 20 min. A solution of 1-naphthaldehyde (1.56 mL, 11.5 mmol) in THF (15 mL) was then added, and the reaction was warmed to rt and stirred for 16 hr. Water (50 mL) was added and the reaction mixture was extracted into Et₂O (3×50 mL). The combined organic fractions were dried (MgSO₄), concentrated in vacuo and chromatographed on silica gel eluting with EtOAc:hexanes 1:9. The product eluted was dissolved in DCM (50 mL) and Dess-Martin periodinane (3.30 g, 7.8 mmol) was added. The reaction was stirred at rt for 16 hr. The reaction mixture was diluted with DCM (50 mL) and water (100 mL) was added. The precipitate was filtered and the layers separated. The aqueous layer was extracted into DCM (2×50 mL), the combined organic fractions were dried (MgSO₄), concentrated in vacuo and chromatographed on silica gel eluting with EtOAc:hexanes 1:9 affording the title compound. δ_{H} (CDCl₃): 0.17 (1H, m), 0.40 (1H, m), 0.63 (1H, m), 0.73 (1H, m), 1.58 (1H, m), 3.60 (1H, d), 3.72 (3H, s), 7.50 (1H, m), 7.56 (1H, m), 7.61 (1H, m), 7.82 (1H, d), 7.89 (1H, d), 8.00 (1H, d), 8.55 (1H, d).

### Preparation 53

### 2-Cyclopropyl-1-naphthalen-1-ylethanone

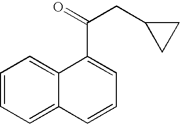

A suspension of cyclopropyl-3-naphthalen-1-yl-3-oxo propionic acid methyl ester (Preparation 52, 0.61 g, 2.3 mmol) and NaCl (0.13 g, 2.3 mmol) in DMSO (15 mL) and water (0.5 mL) was heated to 160° C. for 24 hr. The reaction mixture was cooled to rt and partitioned between water (50 mL) and EtOAc (3×40 mL). The combined organic fractions were dried (MgSO₄), concentrated in vacuo and chromatographed on silica gel eluting with EtOAc:hexanes 1:9 affording the title compound. δ_{H} (CDCl₃): 0.23 (2H, m), 0.61 (2H, m), 1.19 (1H, m), 2.97 (2H, d), 7.50 (1H, m), 7.54 (1H, m), 7.60 (1H, m), 7.82 (1H, m), 7.89 (1H, d), 7.98 (1H, d), 8.60 (1H, d).

### Preparation 54

### 1-Bromo-5-chloronaphthalene

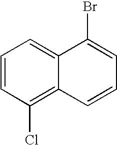

### Preparation 52

### 2-Cyclopropyl-3-naphthalen-1-yl-3-oxopropionic acid methyl ester

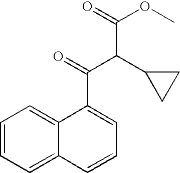

n-Butyllithium (2.5M, 8.3 mL, 20.9 mmol) was added to a solution of diisopropylamine (2.9 mL, 20.9 mmol) in THF (20 mL) at 0° C. under an argon atmosphere. The reaction mixture was stirred at 0° C. for 1 hr, and then cooled to −78° C. A solution of cyclopropylacetic acid methyl ester (Preparation 51, 1.19 g, 10.4 mmol) in THF (15 mL) was added dropwise and the reaction was stirred at −78° C. for 20 min. A solution of 1-naphthaldehyde (1.56 mL, 11.5 mmol) in THF (15 mL) was then added, and the reaction was warmed to rt and stirred for 16 hr. Water (50 mL) was added and the reaction mixture was extracted into Et₂O (3×50 mL). The combined organic fractions were dried (MgSO₄), concentrated in vacuo and chromatographed on silica gel eluting with EtOAc:hexanes 1:9. The product eluted was dissolved in DCM (50 mL) and Dess-Martin periodinane (3.30 g, 7.8 mmol) was added. The reaction was stirred at rt for 16 hr. The reaction mixture was diluted with DCM (50 mL) and water (100 mL) was added. The precipitate was filtered and the layers separated. The aqueous layer was extracted into DCM (2×50 mL), the combined organic fractions were dried (MgSO₄), concentrated in vacuo and chromatographed on silica gel eluting with EtOAc:hexanes 1:9 affording the title compound. δ_{H} (CDCl₃): 0.17 (1H, m), 0.40 (1H, m), 0.63 (1H, m), 0.73 (1H, m), 1.58 (1H, m), 3.60 (1H, d), 3.72 (3H, s), 7.50 (1H, m), 7.56 (1H, m), 7.61 (1H, m), 7.82 (1H, d), 7.89 (1H, d), 8.00 (1H, d), 8.55 (1H, d).

### Preparation 53

### 2-Cyclopropyl-1-naphthalen-1-ylethanone

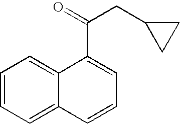

A suspension of cyclopropyl-3-naphthalen-1-yl-3-oxo propionic acid methyl ester (Preparation 52, 0.61 g, 2.3 mmol) and NaCl (0.13 g, 2.3 mmol) in DMSO (15 mL) and water (0.5 mL) was heated to 160° C. for 24 hr. The reaction mixture was cooled to rt and partitioned between water (50 mL) and EtOAc (3×40 mL). The combined organic fractions were dried (MgSO₄), concentrated in vacuo and chromatographed on silica gel eluting with EtOAc:hexanes 1:9 affording the title compound. δ_{H} (CDCl₃): 0.23 (2H, m), 0.61 (2H, m), 1.19 (1H, m), 2.97 (2H, d), 7.50 (1H, m), 7.54 (1H, m), 7.60 (1H, m), 7.82 (1H, m), 7.89 (1H, d), 7.98 (1H, d), 8.60 (1H, d).

### Preparation 54

### 1-Bromo-5-chloronaphthalene

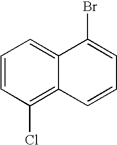

The title compound was prepared according to the method of Robert F. O'Malley et al, J. Org. Chem., 1994, 59, 7335-7340.

### Preparation 55

### 5-chloronaphthalene-1-carbaldehyde

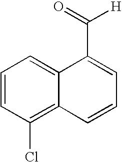

A solution of 1-bromo-5-chloronaphthalene (Preparation 54, 1 g, 4.14 mmol) in THF (20 mL) was cooled to −78° C. under an argon atmosphere. n-Butyllithium (2.5M, 1.8 mL, 4.55 mmol) was added dropwise and the reaction stirred at −78° C. for 1 hr. DMF (0.96 mL, 12.4 mmol) was added and the reaction stirred at −78° C. for 2 hr, then warmed to rt and quenched with 1M HCl (30 mL). The reaction mixture was extracted into EtOAc (3×30 mL) and the combined organic fractions were dried (MgSO₄), concentrated in vacuo and chromatographed on silica gel eluting with EtOAc:hexanes 1:9 affording the title compound. δ_{H} (CDCl₃): 7.60 (1H, m), 7.70 (1H, d), 7.75 (1H, m), 8.05 (1H, d), 8.61 (1H, d), 9.22 (1H, d), 10.41 (1H, s).

### Preparation 56

### 3-(5-chloronaphthalen-1-yl)-2-cyclopropyl-3-oxopropionic acid methyl ester

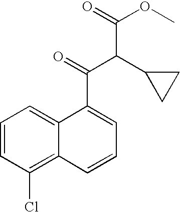

The title compound was prepared using an identical method to that for Preparation 52 starting from 5-chloronaphthalene-1-carbaldehyde (Preparation 55). δ_{H} (CDCl₃): 0.15 (1H, m), 0.40 (1H, m), 0.62 (1H, m), 0.73 (1H, m), 1.54 (1H, m), 3.56 (1H, d), 7.51 (1H, m), 7.62 (1H, m), 7.66 (1H, m), 7.84 (1H, d), 8.40 (1H, d), 8.50 (1H, d).

### Preparation 57

### 1-(5-Chloronaphthalen-1-yl)-2-cyclopropylethanone

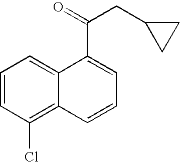

The title compound was prepared using an identical method to that for Preparation 53 starting from 3-(5-chloronaphthalen-1-yl)-2-cyclopropyl-3-oxopropionic acid methyl ester (Preparation 56). δ_{H} (CDCl₃): 0.22 (2H, m), 0.60 (2H, m), 1.16 (1H, m), 2.95 (2H, d), 7.49 (1H, m), 7.62 (2H, m), 7.83 (1H, m), 8.47 (2H, m).

### Preparation 58

### (3,3-Diethoxy-1-methylpropyl)triphenylphosphonium bromide

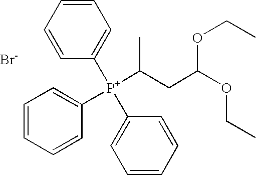

The title compound was prepared according to the method of Henri Brunner et al., Synthesis, 1997, 79-86.

### Preparation 59

### 1-Chloro-7-methylnaphthalene

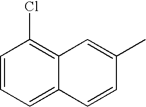

(3,3-Diethoxy-1-methylpropyl)triphenylphosphonium bromide (30 g, 61.6 mmol) was suspended in THF (90 mL) under an argon atmosphere and cooled to −78° C. n-Butyllithium (2.5M, 27 mL, 67.7 mmol) was added dropwise over 15 min, and the reaction stirred at −78° C. for 1.5 hr. A solution of 2-chlorobenzaldehyde (6.9 mL, 61.6 mmol) in THF (10 mL) was added and the reaction warmed to rt and stirred for 16 hr. EtOH (20 mL) and triethylamine (2 mL) were added and the reaction mixture partitioned between water (100 mL) and Et₂O (3×100 mL). The combined organic fractions were dried MgSO₄) and concentrated in vacuo. The residue was dissolved in glacial acetic acid (100 mL) and 48% HBr in water (100 mL) and heated to 100° C. for 16 hr. The reaction was cooled to rt and partitioned between water (150 mL) and Et₂O (3×100 mL). The combined organic fractions were dried (MgSO₄), concentrated in vacuo and chromatographed on silica gel eluting with hexanes affording the title compound. δ_{H} (CDCl₃): 2.59 (3H, s), 7.33 (1H, m), 7.39 (1H, m), 7.56 (1H, m), 7.73 (1H, d), 7.77 (1H, d), 8.07 (1H, s).

### Preparation 60

### 8-Chloronaphthalene-2-carbaldehyde

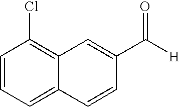

To a refluxing solution of N-bromosuccinimide (1.20 g, 6.7 mmol) and AIBN (84 mg, 0.5 mmol) in CCl₄ (25 mL) was added a solution of 1-chloro-7-methylnaphthalene (Preparation 59, 1.13 g, 6.4 mmol) in CCl₄ (25 mL). The reaction was refluxed for 3 hr, then cooled to rt and the solid filtered and discarded. The filtrate was concentrated in vacuo and dissolved in CHCl₃ (50 mL). Hexamethylenetetramine (1.08 g, 7.7 mmol) was added and the reaction was refluxed for 3 hr, then stirred at rt for 72 hr. The reaction mixture was concentrated in vacuo and the residue dissolved in acetic acid:water (1:1, 40 mL) and concentrated HCl (10 mL). The reaction mixture was refluxed for 3 hr, then cooled to rt and partitioned between water (100 mL) and EtOAc (3×100 mL). The combined organic fractions were dried (MgSO₄), concentrated in vacuo and chromatographed on silica gel eluting with EtOAc:hexanes 1:9 affording the title compound. δ_{H} (CDCl₃): 7.56 (1H, t), 7.68 (1H, m), 7.84 (1H, d), 7.98 (1H, d), 8.03 (1H, dd), 8.77 (1H, s), 1023 (1H, s).

### Preparation 61

### 8-Chloronaphthalene-2-carbonitrile

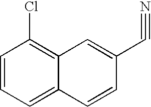

A solution of 8-chloronaphthalene-2-carbaldehyde (Preparation 60, 117 mg, 0.61 mmol) and hydroxylamine hydrochloride (51 mg, 0.74 mmol) in formic acid (5 mL) was heated to reflux for 24 hr. The reaction was cooled to rt and partitioned between water (30 mL) and EtOAc (3×30 mL). The combined organic fractions were dried (MgSO₄), concentrated in vacuo and chromatographed on silica gel eluting with EtOAc:hexanes 1:9 affording the title compound. δ_{H} (CDCl₃): 7.57 (1H, m), 7.70 (2H, m), 7.83 (1H, d), 7.98 (1H, d), 8.69 (1H, s).

### Preparation 62

### 3-Dimethylamino-1-naphthalen-1-ylpropan-1-one hydrochloride

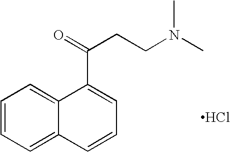

To a solution of 1-acetonaphthone (10 g, 59 mmol), paraformaldehyde (2.3 g, 78 mmol) and dimethylamine hydrochloride (6.36 g, 78 mmol) in ethanol (20 mL) was added concentrated HCl (0.5 mL). The reaction was heated to reflux for 10 hr, then cooled to rt and concentrated in vacuo. The residue was triturated with Et₂O (20 mL) affording the title compound. m/z (ES⁺)=228 [M+H]⁺; RT=2.22 min.

### Preparation 63

### 1-Naphthalen-1-ylpropenone

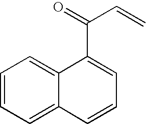

3-Dimethylamino-1-naphthalen-1-ylpropan-1-one hydrochloride (Preparation 62, 15.47 g, 59 mmol) was dissolved in water (400 mL) and basified to pH9 with saturated Na₂CO₃ solution. The free base was extracted into EtOAc (3×150 mL), and the combined organic fractions were dried (MgSO₄) and concentrated in vacuo. The residue was dissolved in MeOH (25 mL) and cooled to 0° C. and iodomethane (8.4 mL, 135 mmol) was added. The reaction mixture was warmed to rt and stirred for 1 hr. The resulting solid was filtered and washed with Et₂O (2×20 mL). The solid was stirred vigorously between Et₂O (100 mL) and saturated NaHCO₃ solution (100 mL) for 16 hr. The layers were separated and the aqueous extracted into EtOAc (2×100 mL). The combined organic fractions were washed with 1M HCl (100 mL), brine (150 mL), dried (MgSO₄) and concentrated in vacuo affording the title compound. δ_{H} (CDCl₃): 6.06 (1H, d), 6.28 (1H, d), 6.97 (1H, dd), 7.55 (3H, m), 7.74 (1H, m), 7.91 (1H, m), 8.00 (1H, d), 8.35 (1H, m).

### Preparation 64

### 3-Benzyloxy-1-naphthalen-1-ylpropan-1-one

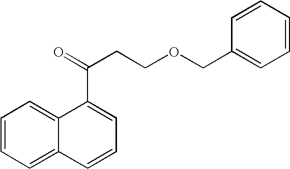

1-Naphthalen-1-ylpropenone Preparation 63, 3.78 g, 20.7 mmol) and benzyl alcohol (3.35 g, 31 mmol) were dissolved in DCM (40 mL) and to this was added concentrated H₂SO₄ (4 drops). The reaction was stirred at rt for 16 hr, then diluted with DCM (40 mL) and washed with saturated NaHCO₃ solution (50 mL) and water (50 mL). The organic layer was dried (MgSO₄), concentrated in vacuo and chromatographed on silica gel eluting with EtOAc:hexanes 1:9. The product was further chromatographed on silica gel eluting with DCM affording the title compound. δ_{H} (CDCl₃): 3.37 (2H, t), 3.99 (2H, t), 4.57 (2H, s), 7.32 (4H, m), 7.56 (4H, m), 7.90 (2H, m), 8.00 (1H, d), 8.63 (1H, d).

### Preparation 65

### 3-Benzyloxy-2-bromo-1-naphthalen-1-ylpropan-1-one

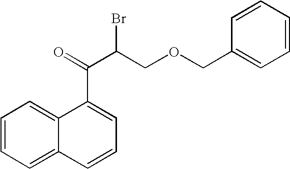

3-Benzyloxy-1-naphthalen-1-ylpropan-1-one (Preparation 64, 2.89 g, 9.9 mmol) and PTAT (4.12 g, 10.9 mmol) were dissolved in THF (40 mL) and stirred at rt for 72 hr. The precipitate was filtered and washed with THF (10 mL) and discarded. The filtrate was concentrated in vacuo and chromatographed on silica gel eluting with EtOAc:hexanes 1:19 affording the title compound. δ_{H} (DMSO): 3.95 (1H, dd), 4.20 (1H, dd), 4.60 (2H, d), 5.96 (1H, t), 7.31 (4H, m), 7.65 (4H, m), 8.07 (1H, d), 8.22 (2H, m), 8.38 (1H, d).

### Preparations 66-70

The procedure described in Preparation 15 was used to prepare the compounds of Preparations 66-70 from the appropriate nitrile and Grignard reagent

| Prep | Structure | Name | ¹H-NMR data |
|---|---|---|---|
| 66 | 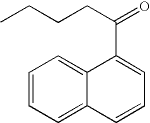 | 1-Naphthalen-1-ylpentan-1- one | δ_{H}(CDCl₃): 0.97 (3 H, t), 1.46 (2 H, m), 1.79 (2 H, m), 3.06 (2 H, t), 7.48-7.61 (3 H, m), 7.85 (1 H, m), 7.89 (1 H, m), 7.98 (1 H, d), 8.55 (1 H, d) |
| 67 | 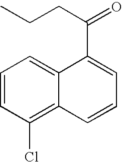 | 1-(5-Chloronaphthalen-1-yl)- butan-1-one | δ_{H}(CDCl₃): 1.04 (3 H, t), 1.83 (2 H, m), 3.03 (2 H, t), 7.48 (1 H, m), 7.64 (2 H, m), 7.87 (1 H, m), 8.43 (1 H, d), 8.48 (1 H, d) |
| 68 | 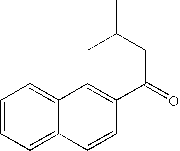 | 3-Methyl-1-naphthalen-2-yl- butan-1-one | δ_{H}(CDCl₃): 1.05 (6 H, d), 2.38 (1 H, m), 2.98 (2 H, d), 7.61 (2 H, m), 7.91 (2 H, m), 7.98 (1 H, d), 8.04 (1 H, m), 8.47 (1 H, s) |
| 69 | 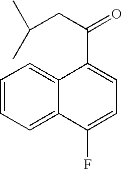 | 1-(4-Fluoronaphthalen-1-yl)- 3-methylbutan-1-one | δ_{H}(CDCl₃): 1.03 (6 H, d), 2.34 (1 H, m), 2.92 (2 H, d), 7.16 (1 H, m), 7.64 (1 H, m), 7.66 (1 H, m), 7.87 (1 H, m), 8.17 (1 H, d), 8.69 (1 H, d) |
| 70 | 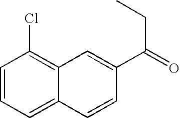 | 1-(8-Chloronaphthalen-2-yl)- propan-1-one | δ_{H}(CDCl₃): 1.32 (3 H, t), 3.20 (2 H, q), 7.50 (1 H, t), 7.64 (1 H, d), 7.80 (1 H, d), 7.92 (1 H, d), 8.11 (1 H, m), 8.89 (1 H, s) |

### Preparations 71-79

The procedure described in Preparation 1 was used to prepare the compounds of Preparations 71-79 from the appropriate ketone.

| Prep | Structure | Name | ¹H-NMR data |
|---|---|---|---|
| 71 | 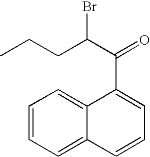 | 2-Bromo-1-naphthalen-1-yl- pentan-1-one | δ_{H}(CDCl₃): 1.02 (3 H, t), 1.54 (1 H, m), 1.63 (1 H, m), 2.17 (1 H, m), 2.28 (1 H, m), 5.23 (1 H, t), 7.52 (1 H, t), 7.57 (1 H, m), 7.64 (1 H, m), 7.89 (2 H, m), 8.03 (1 H, d), 8.47 (1 H, d) |
| 72 | 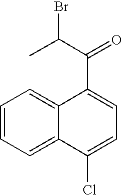 | 2-Bromo-1-(4-chloro- naphthalen-1-yl)propan-1-one | δ_{H}(CDCl₃): 1.99 (3 H, d), 5.33 (1 H, q), 7.63 (1 H, d), 7.69 (2 H, m), 7.77 (1 H, d), 8.38 (1 H, m), 8.45 (1 H, m) |
| 73 | 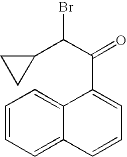 | 2-Bromo-2-cyclopropyl-1- naphthalen-1-ylethanone | δ_{H}(CDCl₃): 0.57 (2 H, m), 0.95 (2 H, m), 1.86 (1 H, m), 4.55 (1 H, d), 7.50 (1 H, t), 7.57 (1 H, t), 7.64 (1 H, t), 7.82 (1 H, d), 7.90 (1 H, d), 8.02 (1 H, d), 8.48 (1 H, d) |
| 74 | 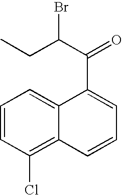 | 2-Bromo-1-(5-chloro- naphthalen-1-yl)butan-1-one | δ_{H}(CDCl₃): 1.15 (3 H, t), 2.19 (1 H, m), 2.34 (1 H, m), 5.12 (1 H, t), 7.52 (1 H, m), 7.64 (2 H, m), 7.89 (1 H, d), 8.32 (1 H, d), 8.52 (1 H, d) |
| 75 | 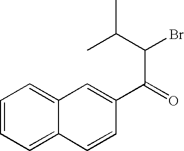 | 2-Bromo-3-methyl-1- naphthalen-2-ylbutan-1-one | δ_{H}(CDCl₃): 1.08 (3 H, d), 1.28 (3 H, d), 2.57 (1 H, m), 5.11 (1 H, d), 7.59 (1 H, m), 7.64 (1 H, m), 7.90 (1 H, d), 7.94 (1 H, d), 8.00 (1 H, d), 8.07 (1 H, m), 8.53 (1 H, s) |
| 76 | 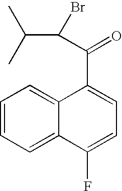 | 2-Bromo-1-(4-fluoro- naphthalen-1-yl)-3-methyl- butan-1-one | δ_{H}(CDCl₃): 1.28 (6 H, m), 2.54 (1 H, m), 4.98 (1 H, d), 7.18 (1 H, m), 7.64 (1 H, m), 7.70 (1 H, m), 7.90 (1 H, m), 8.19 (1 H, d), 8.57 (1 H, d) |
| 77 | 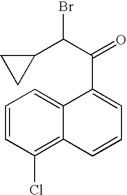 | 2-Bromo-1-(5-chloro- naphthalen-1-yl)-2- cyclopropylethanone | δ_{H}(CDCl₃): 0.57 (2 H, m), 0.96 (2 H, m), 1.83 (1 H, m), 4.51 (1 H, d), 7.53 (1 H, t), 7.62 (1 H, m), 7.67 (1 H, d), 7.83 (1 H, d), 8.33 (1 H, d), 8.51 (1 H, d) |
| 78 | 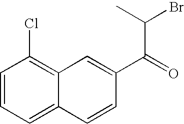 | 2-Bromo-1-(8-chloro- naphthalen-2-yl)propan-1-one | δ_{H}(CDCl₃): 2.00 (3 H, d), 5.52 (1 H, q), 7.53 (1 H, t), 7.66 (1 H, d), 7.82 (1 H, d), 7.95 (1 H, d), 8.13 (1 H, d), 8.99 (1 H, s) |

### Preparation 79

### 6-Methylnaphthalen-1-ylamine

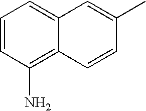

6-Methylnaphthalene-1-carboxylic acid (2.50 g, 13.4 mmol) was mixed with polyphosphoric acid (˜50 mL) and hydroxylamine hydrochloride (990 mg, 14.2 mmol). The mixture was heated to 80° C. and stirred for 30 min. The temperature was slowly raised to 160° C. (froth!) and the stirring was continued for 1 hr before the hot solution was added to a water/ice mixture (˜1.5 L). The resulting solution was washed with EtOAc (200 mL) and then made alkaline with solid NaOH. Extraction with EtOAc (3×300 mL), washing of the combined extracts with water (200 mL) and brine (200 mL), drying (MgSO₄) and concentration in vacuo afforded the title compound. δ_{H} (DMSO): 2.44 (3H, s), 5.63 (2H, br s), 6.60 (1H, d), 7.01 (1H, d), 7.16 (1H, dd), 7.20 (1H, d), 7.50 (1H, s), 7.97 (1H, d); m/z (ES⁺)=158.12 [M+H]⁺; RT=2.31 min.

### Preparation 80

### 1,1-Difluoro-1H-naphthalen-2-one

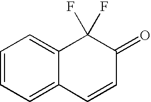

2-Naphthol (4.5 g, 31.21 mmol) was dissolved in DMF (50 mL) and then Selectfluor (22.11 g, 62.42 mmol) was added slowly. The mixture was stirred for 1 hr at rt before the organics were diluted with EtOAc (2×50 mL) and washed with brine (100 mL). The combined extracts were dried (MgSO₄) and concentrated in vacuo to give the title compound. δ_{H} (DMSO): 6.38 (1H, m), 7.60-7.72 (3H, m), 7.89 (2H, m).

### Preparation 81

### 1,1,2,2-Tetrafluoro-1,2-dihydronaphthalene

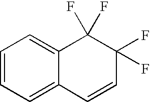

1,1-Difluoro-1H-naphthalen-2-one (Preparation 80, 6.9 g, 31.21 mmol) was dissolved in toluene (5 mL). BF₃.Et₂O (0.44 mL) and then Deoxyfluor (10 mL, 54.61 mmol) were added under an inert atmosphere and the mixture stirred at 60° C. for 2 hr before being cooled to rt and stirred for a further 16 hr. The mixture was cooled to 0° C. and methanol (0.5 mL) added before NaHCO₃ (100 mL) was added dropwise. The organic layer was then diluted with toluene (2×50 mL). The combined extracts were dried (MgSO₄) and concentrated in vacuo to give the title compound. δ_{H} (DMSO): 6.37-6.40 (1H, m), 7.15 (1H, d), 7.54-7.58 (2H, m), 7.68 (1H, t), 7.82 (1H, d).

### Preparation 82

### 1,2 Difluoronaphthalene

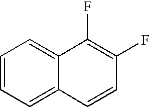

1,1,2,2-Tetrafluoro-1,2-dihydronaphthalene (Preparation 81, 3.9 g, 19.29 mmol) was dissolved in THF (15 mL) and ammonium hydroxide (30 mL) and zinc (6.29 g, 96.30 mmol) were then added. The mixture was stirred at rt under an inert atmosphere for 4 hr then filtered and washed with hexane. The washings were filtered through a short silica column with hexane and concentrated in vacuo to give the title compound. δ_{H} (DMSO): 7.58-7.62 (3H, m), 7.81 (1H, m), 8.01 (2H, t).

### Preparation 83

### 1-(7,8-Difluoronaphthalene-1-yl)propan-1-one

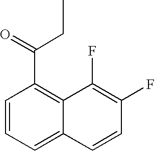

1,2-Difluoronaphthalene (Preparation 82, 1.2 g, 7.3 mmol) was dissolved in DCM (15 mL) and cooled to −15° C. Aluminium chloride (2.9 g, 21.9 mmol) was added portionwise and the mixture was stirred at −15° C. for 15 min. The solution was cooled to −78° C. and propionyl chloride (1.27 mL, 14.6 mmol) was then added dropwise. The mixture was stirred for 16 hr and HCl (20 mL) added slowly. The organics were diluted with EtOAc (100 mL), washed with 1M HCl solution (2×50 mL) dried (MgSO₄) and concentrated in vacuo to afford the title compound. δ_{H} (CDCl₃) 1.25 (3H, t), 2.86 (2H, m), 7.40-7.45 (3H, m), 7.64 (1H, m), 7.90 (1H, d).

### Preparation 84

### 2-Bromo-1-(7,8-difluoronaphthalene-1-yl)propan-1-one

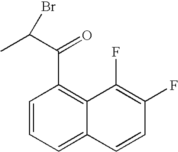

The title compound was prepared from 1-(7,8-difluoronaphthalene-1-yl)propan-1-one (Preparation 83) under similar conditions as described in Preparation 1. δ_{H} (CDCl₃): 2.00 (3H, m), 5.01 (1H, m), 7.25 (1H, q), 7.38 (1H, t), 7.64 (1H, d), 7.73 (1H, m), 7.98 (1H, d).

### Preparation 85

### 4-Bromo-2-fluoro-N-methoxy-N-methylbenzamide

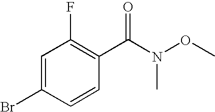

The title compound was prepared from 4-bromo-2-fluorobenzoic acid (9.4 g, 42.9 mmol) under similar conditions as described in Preparation 27. δ_{H} (CDCl₃): 3.40 (3H, s), 3.60 (3H, s), 7.35 (3H, m).

### Preparation 86

### 1-(4-Bromo-2-fluorophenyl)propan-1-one

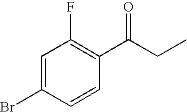

The title compound was prepared from 4-bromo-2-fluoro-N-methoxy-N-methylbenzamide (Preparation 85, 5.62 g, 21.45 mmol) under similar conditions as described in Preparation 28. δ_{H} (CDCl₃): 1.20 (3H, t), 3.00 (3H, q), 7.40 (2H, m), 7.80 (1H, t).

### Preparation 87

### 2-Bromo-1-(4-bromo-2-fluorophenyl)propan-1-one

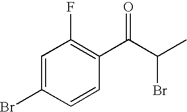

The title compound was prepared from 1-(4-bromo-2-fluorophenyl)propan-1-one (Preparation 86, 1.64 g, 7.1 mmol) under similar conditions as described in Preparation 29. δ_{H} (CDCl₃): 1.90 (3H, d), 5.30 (1H, q), 7.35 (1H, d), 7.45 (1H, d), 7.80 (1H, t).

### Preparation 88

### 1-(4-Bromo-3-methylphenyl)propan-1-one

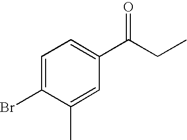

The title compound was prepared from 4-bromo-3-methylbenzonitrile (7.7 g, 39.3 mmol) under similar conditions as described in Preparation 42. δ_{H} (CDCl₃): 1.20 (3H, t), 3.00 (2H, q), 7.60 (2H, s), 7.80 (1H, s).

### Preparation 89

### 2-Bromo-1-(4-bromo-3-methylphenyl)propan-1-one

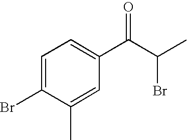

The title compound was prepared from 1-(4-bromo-3-methylphenyl)propan-1-one (Preparation 88, 2.31 g, 10.13 mmol) under similar conditions as described in Preparation 16. δ_{H} (CDCl₃): 1.97 (3H, d), 2.50 (3H, s), 5.25 (1H, q), 7.70 (2H, m), 7.80 (1H, s).

### Preparation 90

### 1-(3,4-Dichlorophenyl)-3-methylbutan-1-one

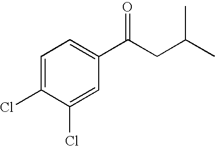

The title compound was prepared from 3,4-dichlorobenzonitrile (5 g, 29.1 mmol) under similar conditions as described in Preparation 42. δ_{H} (CDCl₃): 1.05 (6H, d), 2.30 (1H, m), 2.80 (2H, d), 7.60 (1H, d), 7.80 (1H, d), 8.05 (1H, s).

### Preparation 91

### 2-Bromo-1-(3,4-dichlorophenyl)-3-methylbutan-1-one

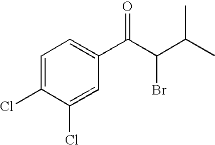

The title compound was prepared from 1-(3,4-dichlorophenyl)-3-methylbutan-1-one (Preparation 90, 1.6 g, 6.9 mmol) under similar conditions as described in Preparation 16. δ_{H} (CDCl₃): 1.05 (3H, d), 1.10 (3H, d), 2.50 (1H, m), 4.80 (1H, d), 7.60 (1H, d), 7.80 (1H, d), 8.15 (1H, s).

### Preparation 92

### 1-(4,5-Difluoronaphthalen-1-yl)propan-1-one

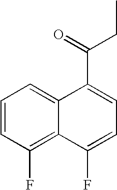

A solution of 1,8-difluoronaphthalene (1 g, 6.1 mmol) (prepared by the procedure of Mallory F. B, J. Amer. Chem. Soc. 1974, 96, 3536) and propionyl chloride (0.54 mL, 6.16 mmol) in DCM (20 mL) was added to a suspension of aluminium trichloride (2.44 g, 18.3 mmol) in DCM (5 mL) at −40° C. After 1 hr the reaction was warmed to rt and quenched with hydrochloric acid 2N HCl (10 mL). The mixture was extracted with EtOAc (3×10 mL). The combined organic layers were washed with sodium hydroxide (2N, 10 mL), dried (MgSO₄) and concentrated in vacuo. Purification by flash-chromatography on silica gel (eluent hexane/EtOAc, 98:2) gave the title compound. δ_{H} (CDCl₃): 1.30 (3H, t), 3.10 (2H, q), 7.20 (2H, m), 7.60 (1H, m), 7.90 (1H, m), 8.50 (1H, d).

### Preparation 93

### 2-Bromo-1-(4,5-difluoronaphthalen-1-yl)propan-1-one

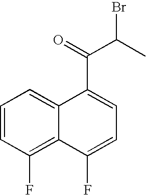

The title compound was prepared from 1-(4,5-difluoronaphthalen-1-yl)propan-1-one (Preparation 92, 0.86 g, 3.91 mmol) under similar conditions as described in Preparation 16. δ_{H} (DMSO): 1.80 (3H, d), 5.90 (1H, q), 7.45 (2H, m), 7.80 (1H, d), 8.20 (1H, d), 8.30 (1H, m).

### Preparation 94

### 1-(4,5-Difluoronaphthalen-1-yl)butan-1-one

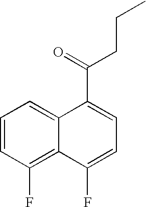

A solution of 1,8-difluoronaphthalene (1.0 g, 6.1 mmol) and butyryl chloride (0.72 g, 6.16 mmol) in DCM (20 mL) was added to a suspension of aluminium trichloride (2.44 g, 18.3 mmol) in DCM (5 mL) at 40° C. After 1 hr the reaction was warmed to rt and quenched with hydrochloric acid 2N HCl (10 mL). The mixture was extracted with EtOAc (3×10 mL). The combined organic layers were washed with sodium hydroxide (2N, 10 mL), dried (MgSO₄) and concentrated in vacuo. Purification by flash-chromatography on silica gel (eluent: hexane/EtOAc 98:2) gave the title compound. δ_{H} (CDCl₃): 1.05 (3H, t), 1.80 (2H, m), 3.00 (2H, t), 7.20 (2H, m), 7.60 (1H, m), 7.90 (1H, m), 8.50 (1H, d).

### Preparation 95

### 2-Bromo-1-(4,5-difluoronaphthalen-1-yl)butan-1-one

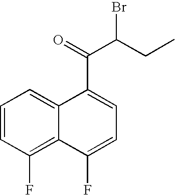

The title compound was prepared from 1-(4,5-difluoronaphthalen-1-yl)butan-1-one (Preparation 94, 0.8 g, 3.41 mmol) under similar conditions as described in Preparation 16. δ_{H} (CDCl₃): 1.20 (3H, t), 2.2 (1H, m), 2.4 (1H, m), 5.1 (1H, t), 7.2 (2H, m), 7.6 (1H, m), 7.9 (1H, m), 8.3 (1H, d).

### Preparation 96

### 1-(4,5-Dichloronaphthalen-1-yl)propan-1-one

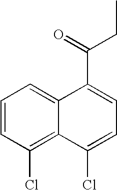

The title compound was prepared from 1,8-dichloronaphthalene (0.8 g, 4.1 mmol) (prepared by the procedure of Hodgson J. Chem. Soc. 1947, 80) under similar conditions as described in Preparation 92. δ_{H} (CDCl₃): 1.30 (3H, t), 3.00 (2H, q), 7.50 (1H, t), 7.60-7.80 (3H, m), 8.40 (1H, d).

### Preparation 97

### 2-Bromo-1-(4,5-dichloronaphthalen-1-yl)propan-1-one

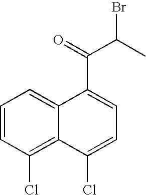

The title compound was prepared from 1-(4,5-dichloronaphthalen-1-yl)propan-1-one (Preparation 96, 0.84 g, 2.53 mmol) under similar conditions as described in Preparation 16. δ_{H} (CDCl₃): 2.00 (3H, d), 525 (1H, q), 7.45 (1H, t), 7.60-7.80 (3H, m), 8.20 (1H, d).

### Preparation 98

### 1-(5,7-Dichloronaphthalen-1-yl)propan-1-one

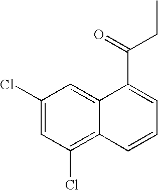

5,7-Dichloronaphthalene-1-carboxylic acid (1.5 g, 6.25 mmol) (Sestanj, Kazinmir Eur. Pat Appl. (1982), EP 59596 A1 19820908), oxalyl chloride (0.6 mL, 6.87 mmol), DMF (1 drop) were combined in DCM (50 mL) and stirred for 12 hr. The mixture was concentrated in vacuo, solubilised in THF (40 mL) and cooled to −78° C. Iron(III)acetylacetonate (66 mg, 0.19 mmol) was added followed by dropwise addition of ethylmagnesium bromide (2.7 mL, 3M solution, 8.12 mmol). After 1 hr the reaction was warmed to rt and saturated ammonium chloride solution (30 mL) was added. The mixture was extracted with DCM (3×50 mL) and the combined organics dried (MgSO₄) and concentrated in vacuo. Purification by flash-chromatography on silica gel (eluent: hexane/ethyl acetate 99:1) gave the title compound. δ_{H} (CDCl₃): 1.10 (3H, t), 3.00 (2H, q), 7.60 (2H, m), 7.09 (1H, d), 8.40 (1H, d), 8.50 (1H, s).

### Preparation 99

### 2-Bromo-1-(5,7-dichloronaphthalen-1-yl)propan-1-one

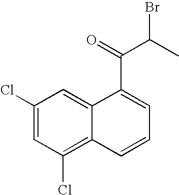

The title compound was prepared from 1-(5,7-dichloronaphthalen-1-yl)propan-1-one (Preparation 98, 0.719 g, 2.84 mmol) under similar conditions as described in Preparation 16. δ_{H} (CDCl₃): 2.00 (3H, d), 5.40 (1H, q), 7.70 (2H, m), 8.00 (1H, d), 8.42 (1H, s), 8.50 (1H, d).

### Preparations 100-106

The procedure described in Preparation 18 was used to prepare the compounds of Preparations 100-104 and the procedure described in Preparation 23 was used to prepare the compounds of Preparations 105 and 106.

| Prep | Structure | Name | LCMS | ¹H-NMR |
|---|---|---|---|---|
| 100 | 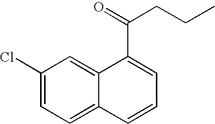 | 1-(7- Chloronaphthalen- 1-yl)butan-1-one | m/z (ES⁺) = 233.10 [M + H]⁺; RT = 4.18 min | δ_{H}(CDCl₃): 1.08 (3 H, t), 1.86 (2 H, m), 3.07 (2 H, t), 7.50-7.56 (2 H, m), 7.84 (1 H, d), 7.95- 8.00 (2 H, m), 8.74 (1 H, s) |
| 101 | 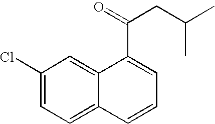 | 1-(7- Chloronaphthalen- 1-yl)-3- methylbutan-1- one | m/z (ES⁺) = 247.08 [M + H]⁺; RT = 4.32 min | δ_{H}(CDCl₃): 1.07 (6 H, d), 2.34-2.41 (1 H, m), 2.96 (2 H, d), 7.51-7.56 (2 H, m), 7.84 (1 H, d), 7.94 (1 H, d), 7.98 (1 H, d), 8.72 (1 H, s) |
| 102 | 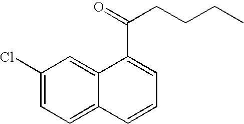 | 1-(7- Chloronaphthalen- 1-yl)pentan-1- one | m/z (ES⁺) = 247.09 [M + H]⁺; RT = 4.39 min | δ_{H}(CDCl₃): 1.00 (3 H, t), 1.45-1.50 (2 H, m), 1.77-1.85 (2 H, m), 3.08 (2 H, t), 7.49-7.54 (2 H, m), 7.82 (1 H, d), 7.96 (2 H, m), 8.73 (1 H, s) |
| 103 | 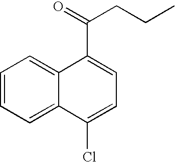 | 1-(4- Chloronaphthalen- 1-yl)butan-1- one | m/z (ES⁺) = 233.14 [M + H]⁺; RT = 4.09 min | δ_{H}(CDCl₃): 1.04 (3 H, t), 1.83 (2 H, m), 3.01 (2 H, t), 7.60 (1 H, d), 7.64- 7.66 (2 H, m), 7.74 (1 H, d), 8.34-8.37 (1 H, m), 8.56 (1 H, m) |
| 104 | 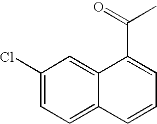 | (7- Chloronaphthalen- 1-yl)ethanone | — | δ_{H}(CDCl₃): 2.77 (3 H, s), 7.50-7.55 (2 H, m), 7.82 (1 H, d), 7.99-8.04 (2 H, m), 8.92 (1 H, s) |
| 105 | 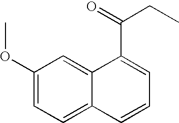 | 1-(7- Methoxynaphthalen- 1-yl)propan- 1-one | — | δ_{H}(CDCl₃): 1.33 (3 H, t), 3.14 (2 H, q), 3.99 (3 H, s), 7.22-7.25 (1 H, m), 7.36-7.40 (1 H, m), 7.79 (1 H, d), 7.94- 7.99 (2 H, m), 8.30 (1 H, s) |
| 106 | 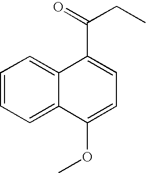 | 1-(4- Methoxynaphthalen- 1-yl)propan-1- one | m/z (ES⁺) = 215.10 [M + H]⁺; RT = 3.76 min | δ_{H}(CDCl₃): 1.31 (3 H, t), 3.09 (2 H, q), 4.07 (3 H, s), 6.79 (1 H, d), 7.53- 7.57 (1 H, t), 7.62- 7.68 (1 H, t), 7.97 (1 H, d), 8.35 (1 H, d), 8.93 (1 H, d) |

### Preparations 107-114

The procedure described in Preparation 16 was used to prepare the compounds of Preparations 107-114 from the appropriate ketone.

| Prep | Structure | Name | ¹H-NMR data |
|---|---|---|---|
| 107 | 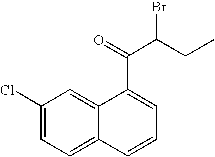 | 2-Bromo-1-(7-chloro- naphthalen-1-yl)butan-1- one | δ_{H}(CDCl₃): 1.17 (3 H, t), 2.18-2.25 (1 H, m), 2.34-2.41 (1 H, m), 5.17 (1 H, t), 7.51- 7.55 (2 H, m), 7.84 (1 H, d), 7.97 (1 H, d), 8.02 (1 H, d), 8.62 (1 H, s) |
| 108 | 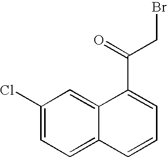 | 2-Bromo-1-(7-chloro- naphthalen-1-yl)ethanone | δ_{H}(CDCl₃): 4.59 (2 H, s), 7.52-7.57 (2 H, m), 7.84 (1 H, d), 8.02-8.06 (2 H, m), 8.80 (1 H, s) |
| 109 | 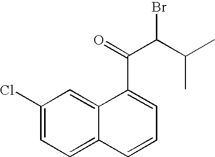 | 2-Bromo-1-(7-chloro- naphthalen-1-yl)-3- methyl-butan-1-one | δ_{H}(CDCl₃): 1.16 (3 H, d), 1.28 (3 H, d), 2.54-2.59 (1 H, m), 5.03 (1 H, d), 7.52-7.56 (2 H, m), 7.85 (1 H, d), 7.96 (1 H, d), 8.03 (1 H, d), 8.59 (1 H, s) |
| 110 | 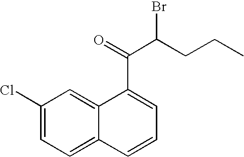 | 2-Bromo-1-(7-chloro- naphthalen-1-yl)pentan- 1-one | δ_{H}(CDCl₃): 1.06 (3 H, t), 1.51-1.69 (2 H, m), 2.18-2.34 (2 H, m), 5.24 (1 H, t), 7.55 (2 H, m), 7.85 (1 H, d), 7.98 (1 H, d), 8.03 (1 H, d), 8.61 (1 H, s) |
| 111 | 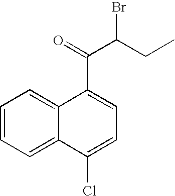 | 2-Bromo-1-(4-chloro- naphthalen-1-yl)butan-1- one | δ_{H}(CDCl₃): 1.16 (3 H, t), 2.15-2.23 (1 H, m), 2.32-2.39 (1 H, m), 5.11 (1 H, m), 7.62 (1 H, d), 7.68-7.72 (2 H, m), 7.78 (1 H, d), 8.37-8.39 (1 H, m), 8.46-8.48 (1 H, m) |
| 112 | 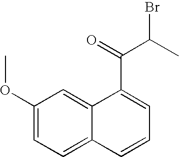 | 2-Bromo-1-(7-methoxy- naphthalen-1-yl)propan- 1-one | δ_{H}(CDCl₃): 2.01 (3 H, d), 4.00 (3 H, s), 5.47 (1 H, q), 7.24-7.27 (1 H, m), 7.40 (1 H, t), 7.81 (1 H, d), 7.97-8.00 (2 H, m), 8.12 (1 H, s) |
| 113 | 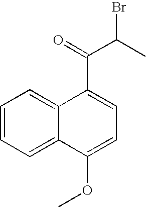 | 2-Bromo-1-(4-methoxy- naphthalen-1-yl)propan- 1-one | δ_{H}(CDCl₃): 1.99 (3 H, d), 4.11 (3 H, s), 5.46 (1 H, q), 6.84 (1 H, d), 7.57 (1 H, t), 7.68 (1 H, t), 8.04 (1 H, d), 8.37 (1 H, d), 8.81 (1 H, d) |
| 114 | 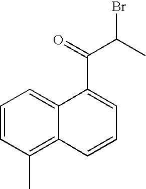 | 2-Bromo-1-(5-methyl- naphthalen-1-yl)propan- 1-one | δ_{H}(CDCl₃): 2.01 (3 H, d), 2.76 (3 H, s), 5.38 (1 H, q), 7.44 (1 H, d), 7.51-7.60 (2 H, m), 7.85 (1 H, d), 8.22-8.25 (2 H, m) |

### Preparation 115

### 5-Methylnaphthalene-1-carboxylic acid tert-butyl ester

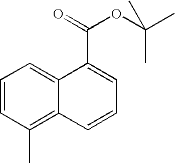

To a solution of 5-bromo-1-naphthalene-1-carboxylic acid tert-butyl ester (J. Org. Chem.; 2002; 67(4); 1171-1177) (1.00 g, 3.26 mmol) in THF (10 mL) at −78° C. was added butyl lithium (2.5M, 1.56 mL) over a period of 2 min. After stirring for 40 min methyl iodide (0.55 g) was added and the reaction allowed to warm to rt over 1.5 hr. The reaction was quenched water (10 mL) and partitioned between water and diethyl ether (3×50 mL). The combined extracts were dried (MgSO₄) and concentrated. Purification of the residue by flash-chromatography (eluent: isohexane/EtOAc: 98:2) gave the title compound. δ_{H} (CDCl₃): 1.70 (9H, s), 2.78 (3H, s), 7.37 (d, 1H), 7.46-7.54 (m, 2H), 8.04 (d, 1H), 8.18 (d, 1H), 8.66 (d, 1H); m/z (ES⁺)=242.23 [M+H]⁺; RT=2.86 min.

### Preparation 116

### 5-Methylnaphthalene-1-carboxylic acid

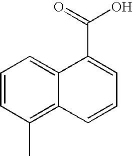

5-Methylnaphthalene-1-carboxylic acid tert-butyl ester (Preparation 115, 1.37 g, 4.45 mmol) was dissolved in DCM (10 mL) and TFA (3 mL) was added. After stirring for 16 hr, the reaction was concentrated in vacuo to give the title compound. δ_{H} (DMSO): 2.71 (3H, s), 7.45 (1H, d), 7.53 (1H, m), 7.64 (1H, m), 8.13 (1H, d), 8.28 (1H, d), 8.68 (1H, d).

### Preparation 117

### 1-(5-Methylnaphthalen-1-yl)propan-1-one

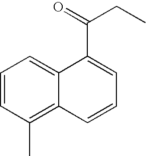

The title compound was prepared from 5-methylnaphthalene-1-carboxylic acid (Preparation 116) via acid chloride under similar conditions as described in Preparation 98. δ_{H} (CDCl₃): 1.20 (3H, t), 2.63 (3H, s), 2.97 (2H, q), 7.29 (1H, d), 7.35-7.46 (2H, m), 7.69 (1H, d), 8.07 (1H, d), 8.22 (1H, d); m/z (ES⁺)=199.07 [M+H]⁺; RT=3.86 min.

### Preparation 118

### 5-Methoxynaphthalene-1-carboxylic acid methyl ester

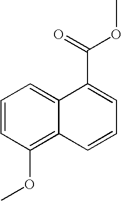

The title compound was prepared according to the method of Can. J. Chem. (2004), 240-253.

### Preparation 119

### 5-Methoxynaphthalene-1-carboxylic acid

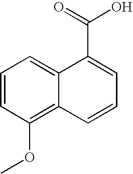

5-Methoxynaphthalene-1-carboxylic acid methyl ester (Preparation 118, 1.43 g) was dissolved in methanol (30 mL) and 2M sodium hydroxide solution added and stirred for 16 hr then concentrated in vacuo. 1M NaOH (50 mL) was added, washed EtOAc (50 mL), acidified using concentrated HCl then extracted into DCM (3×50 mL). The combined organic fractions were dried (MgSO₄) and concentrated in vacuo to afford the title compound. δ_{H} (DMSO): 4.00 (3H, s), 7.06 (1H, d), 7.60 (2H, m), 8.16 (1H, d), 8.42 (2H, m), 13.10 (1H, s).

### Preparation 120

### 1-(5-Methoxynaphthalen-1-yl)propan-1-one

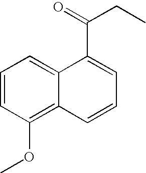

The title compound was prepared from 5-methoxynaphthalene-1-carboxylic acid (Preparation 119) via the acid chloride as described in Preparation 98. δ_{H} (DMSO): 1.18 (3H, t), 3.10 (2H, q), 3.98 (3H, s), 7.08 (1H, d), 7.55 (2H, m), 7.96 (1H, d), 8.04 (1H, d), 8.40 (1H, d).

### Preparation 121

### 2-Bromo-1-(5-methoxynaphthalen-1-yl)propan-1-one

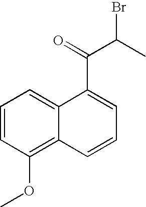

The title compound was prepared from 1-(5-methoxynaphthalen-1-yl)propan-1-one (Preparation 120) under conditions described in Preparation 16. δ_{H} (CDCl₃): 2.01 (3H, d), 4.04 (3H, s), 5.45 (1H, q), 7.14 (1H, d), 7.48 (2H, m), 7.98 (1H, d), 8.10 (1H, d), 8.40 (1H, d).

### Preparation 122

### 1-(5-Chloronaphthalen-1-yl)ethanol

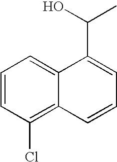

The title compound was prepared from 5-chloronaphthalene-1-carbaldehyde (Preparation 55) with methyl magnesium chloride under similar conditions as described in Preparation 187. δ_{H} (CDCl₃): 1.75 (3H, d), 5.70 (1H, q), 7.45 (1H, m), 7.42 (1H, t), 7.60 (2H, m), 7.78 (2H, d), 8.06 (1H, d), 8.27 (1H, d).

### Preparation 123

### 1-(5-Chloronaphthalen-1-yl)ethanone

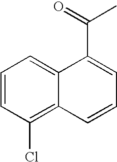

The title compound was prepared from 1-(5-chloronaphthalen-1-yl)ethanol (Preparation 122) under similar conditions as described in Preparation 129. δ_{H} (CDCl₃): 2.75 (3H, s), 7.55 (1H, t), 7.72 (2H, m), 8.04 (1H, m), 8.56 (1H, d), 8.70 (1H, d).

### Preparation 124

### 2-Bromo-1-(5-Chloronaphthalen-1-yl)ethanone

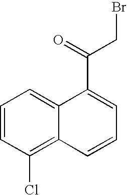

The title compound was prepared from 1-(5-chloronaphthalen-1-yl)ethanone (Preparation 123) under similar conditions as described in Preparation 19. δ_{H} (CDCl₃): 4.60 (2H, d), 7.63 (1H, m), 7.74 (3H, m), 8.04 (2H, m).

### Preparation 125

### 2-Bromo-1-(5,6,7,8-tetrahydronaphthalen-2-yl)ethanone

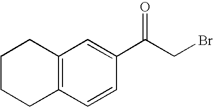

The title compound was prepared from 1-(5,6,7,8-tetrahydronaphthalen-2-yl)ethanone under similar conditions as described in Preparation 44. δ_{H} (DMSO): 1.77 (4H, m), 2.80 (4H, m), 4.87 (2H, s), 7.24 (1H, d), 7.72 (2H, m).

### Preparation 126

### 3-(5,6,7,8-Tetrahydronaphthalen-2-yl)-5,6-dihydroimidazo[2,1-b]thiazole hydrobromide

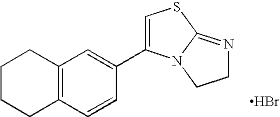

The title compound was prepared from 2-bromo-1-(5,6,7,8-tetrahydronaphthalen-2-yl)ethanone (Preparation 125) and 2-imidazolidinethione under similar conditions as described in Example 66. γ_{H} (DMSO): 1.77 (4H, m), 2.80 (4H, m), 4.30 (2H, t), 4.53 (2H, t), 6.95 (1H, s), 7.24 (1H, d), 7.34 (2H, d), 9.61 (1H, br); m/z (ES⁺)=257.07 [M+H]⁺; RT=2.51 min.

### Preparation 127

### 2-Bromo-3-(5,6,7,8-tetrahydronaphthalen-2-yl)-5,6-dihydroimidazo[2,1-b]thiazole hydrobromide

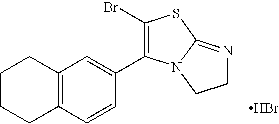

The title compound was prepared from 3-(5,6,7,8-tetrahydronaphthalen-2-yl)-5,6-dihydroimidazo[2,1-b]thiazole hydrobromide (Preparation 126) under similar conditions as described in Example 11. δ_{H} (DMSO): 1.79 (4H, m), 2.80 (4H, m), 4.23 (2H, m), 4.32 (2H, m), 7.29 (3H, m), 9.60 (1H, br).

### Preparation 128

### 1-(5-Trifluoromethylnaphthalen-1-yl)propan-1-ol

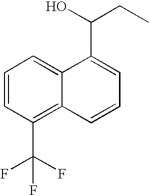

1-Bromo-5-trifluoromethylnaphthalene (4.0 g, 14.54 mmol) was dissolved in THF (80 mL) and cooled under inert atmosphere at −78° C. n-BuLi (6.4 mL, 2.5M solution) was then added dropwise over 10 min and the solution allowed to stir for further 2 hr. Propionaldehyde (3.2 mL, 43.62 mmol) was then added slowly over 5 min and the mixture allowed to stir for 1 hr. The cooling bath was removed and the mixture was stirred for 16 hr at rt before a 2M HCl solution (50 mL) was added. The mixture was stirred for 5 min then the aqueous layer was extracted with EtOAc (3×60 mL). The combined organic layers were washed (brine), dried (MgSO₄) and concentrated in vacuo. Purification by flash-chromatography on silica gel (eluent: hexane/EtOAc: 5/1 then 4/1) gave the title compound. δ_{H} (CDCl₃): 1.05 (3H, t), 1.90-2.05 (2H, m), 5.45 (1H, m), 7.55 (1H, t), 7.65 (1H, t), 7.78 (1H, d), 7.90 (1H, d), 8.18 (1H, d), 8.40 (1H, d).

### Preparation 129

### 1-(5-Trifluoromethylnaphthalen-1-yl)propan-1-one

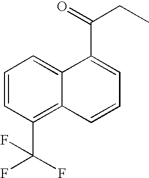

1-(5-Trifluoromethylnaphthalen-1-yl)propan-1-ol (Preparation 128, 2.5 g, 9.832 mmol) was dissolved in DCM (80 mL) and Dess-Martin reagent was added portionwise under inert atmosphere at rt. The mixture was stirred for 16 hr at rt before sat. NaHCO₃ solution (80 mL) was added, extracted with DCM, dried (MgSO₄) and concentrated in vacuo. Purification by flash-chromatography on silica gel (eluent: DCM) gave the title compound. δ_{H} (DMSO): 1.20 (3H, t), 3.17 (2H, q), 7.75 (1H, t), 7.85 (1H, t), 8.10 (1H, d), 8.20 (1H, d), 8.30 (1H, br d), 8.60 (1H, d).

### Preparation 130

### 2-Bromo-1-(5-trifluoromethylnaphthalen-1-yl)propan-1-one

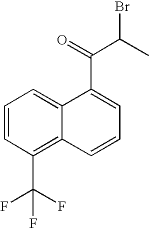

The title compound was prepared from 1,1-(5-trifluoromethylnaphthalen-1-yl)propan-1-one (Preparation 129) under similar conditions as described in Preparation 16. δ_{H} (DMSO): 1.95 (3H, d), 5.95 (1H, q), 7.80-7.90 (2H, m), 8.15 (1H, d), 8.25-8.35 (2H, m), 8.50 (1H, d).

### Preparation 131

### 2-Fluoronaphthalene

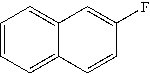

6-Fluoronaphthalene-1-carboxylic acid (15.0 g, 78.87 mmol) was suspended in Quinidine (35 mL) and Cu(0) powder (8.8 g, 138.03 mmol) was added under inert atmosphere and the reaction was heated under reflux for 48 hr. The mixture was allowed to cool to rt and filtered, the filter cake was washed with EtOH (20 mL) then ⁱhexane (20 mL). The washings were combined and concentrated in vacuo. Organics were diluted with EtOAc (200 mL), washed with 2M HCl solution (2×50 mL) then brine (100 mL), dried (MgSO₄) and concentrated in vacuo. Purification by flash-chromatography on silica gel (eluent: hexane) gave the title compound. δ_{H} (DMSO): 7.40-7.60 (3H, m), 7.72 (1H, d), 7.90-8.10 (3H, m).

### Preparation 132

### 1-(7-Fluoronaphthalen-1-yl)propan-1-one

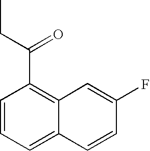

2-Fluoronaphthalene (Preparation 131, 8.0 g, 55.10 mmol) was dissolved in DCM (120 mL) under inert atmosphere and the reaction was cooled at 0° C. AlCl₃ (22.8 g, 170.82 mmol) was then added in one portion and the mixture stirred for 15 min. The mixture was then cooled at −78° C. and propionyl chloride (9.6 mL, 110.2 mmol) was added slowly over 15 min. The mixture was then stirred for 16 hr and allowed over this period to reach rt. Organics were diluted with EtOAc (200 mL), washed with 1M HCl solution (2×50 mL) dried (MgSO₄) and concentrated in vacuo to afford the title compound. δ_{H} (DMSO): 1.15 (3H, t), 3.15 (2H, q), 7.52 (1H, m), 7.61 (1H, m), 8.15 (1H, m), 8.25 (2H, m), 8.35 (1H, m).

### Preparation 133

### 2-Bromo-1-(7-fluoronaphthalen-1-yl)propan-1-one

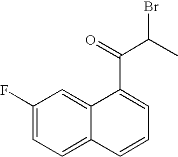

The title compound was prepared from 1⇓-fluoronaphthalen-1-yl)propan-1-one (Preparation 132, 11.7 g, 55.10 mmol) under similar conditions as described in Preparation 16. δ_{H} (DMSO): 1.90 (3H, m), 5.95 (1H, q), 7.55-7.70 (2H, m), 8.10-8.20 (2H, m), 8.25-8.35 (2H, m).

### Preparation 134

### 1-(7-Fluoronaphthalen-1-yl)butan-1-one

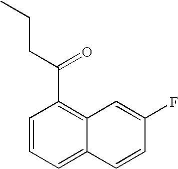

The title compound was prepared from 2-fluoronaphthalene (Preparation 131, 1.0 g, 6.88 mmol) and butyryl chloride under similar conditions as described in Preparation 18. δ_{H} (DMSO): 0.95 (3H, t), 1.70 (2H, m), 3.10 (2H, t), 7.55 (1H, m), 7.65 (1H, m), 8.15 (1H, m), 8.22 (2H, m), 8.30 (1H, m).

### Preparation 135

### 2-Bromo-1-(7-fluoronaphthalen-1-yl)butan-1-one

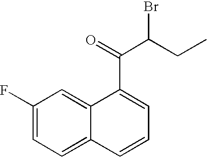

The title compound was prepared from 1-(7-fluoronaphthalen-1-yl)butan-1-one (Preparation 134, 1.47 g, 6.88 mmol) under similar conditions as described in Preparation 16. δ_{H} (DMSO): 1.10 (3H, t), 2.05 (1H, m), 2.15 (1H, m), 5.80 (1H, t), 7.55-7.70 (2H, m), 8.10-8.20 (2H, m), 8.25-8.35 (2H, m).

### Preparation 136

### 3-(3,4-Dichlorophenyl)-5,6-dihydroimidazo[2,1-b]thiazole hydrobromide

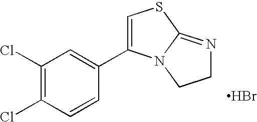

The title compound was prepared from 2-bromo-1-(3,4-dichlorophenyl)ethanone and 2-imidazolidinethione under similar conditions as described in Example 1. δ_{H} (DMSO): 4.30 (2H, m), 4.50 (2H, m), 7.20 (1H, s), 7.60 (1H, d), 7.80 (1H, d), 7.95 (1H, s), 9.60 (1H, br s); m/z (ES⁺)=271.03 [M+H]⁺; RT=2.70 min.

### Preparation 137

### 2-Bromo-3-(3,4-dichlorophenyl)-5,6-dihydroimidazo[2,1-b]thiazole Hydrobromide

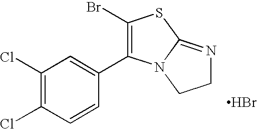

3-(3,4-Dichlorophenyl)-5,6-dihydroimidazo[2,1-b]thiazole hydrobromide (Preparation 136, 5.0 g, 13.85 mmol) was suspended in DCM (100.0 mL) and sat. NaHCO₃ solution (100 mL) was added. The mixture was stirred vigorously for 15 min and the two phases separated; the organic layer was dried by using a phase-separation cartridge and cooled at 0° C. by using an ice bath. Bromine (0.71 mL, 13.85 mmol) was then added over 5 min and the mixture was stirred at 0° C. under an inert atmosphere for 1 hr. After few minutes a yellow precipitate formed, the ice bath was removed and the mixture stirred for 16 hr. The suspension was filtered and the solid washed with diethyl ether (30.0 mL) to afford the title compound. δ_{H} (DMSO): 4.35-4.15 (4H, m), 7.60 (1H, d), 7.90 (2H, m), 9.55 (1H, br s).

### Preparation 138

### 3-(3,4-Dichlorophenyl)-5,6-dihydroimidazo[2,1-b]thiazole-2-carbaldehyde

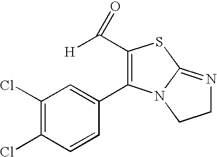

Ethyl magnesium bromide 2.0 M solution in diethyl ether (10.2 mL, 20.36 mmol) was added to a solution of THF (50.0 mL) under inert atmosphere and the mixture cooled at 0° C. by using an ice bath. 2-Bromo-3-(3,4-dichlorophenyl)-5,6-dihydroimidazo[2,1-b]thiazole hydrobromide (Preparation 137, 3.0 g, 6.79 mmol) was added portionwise to the above solution over 5 min. After stirring for 2 hr, DMF (2.10 mL, 27.15 mmol) was added to the solution and the mixture stirred for 16 hr. Saturated ammonium chloride (50 mL) was added slowly to the mixture and then partitioned between EtOAc (150 mL) and saturated sodium chloride (100 mL). The organic layer was dried (MgSO₄) and concentrated in vacuo to afford the title compound. δ_{H} (DMSO): 3.90 (2H, m), 4.20 (2H, m), 7.75 (1H, d), 7.85 (1H, d), 8.10 (1H, s), 9.20 (1H, s).

### Preparation 139

### 2-Bromo-3-(3,4-dichlorophenyl)-3-oxopropionic acid methyl ester

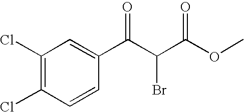

The title compound was prepared from 3-(3,4-dichlorophenyl)-3-oxopropionic acid methyl ester (1.0 g, 4.05 mmol) under similar conditions as described in Preparation 16. δ_{H} (DMSO): 3.80 (3H, s), 6.75 (1H, s), 7.90 (1H, d), 8.00 (1H, d), 8.30 (1H, s).

### Preparation 140

### 3-(3,4-Dichlorophenyl)-5,6-dihydroimidazo[2,1-b]thiazole-2-carboxylic acid methyl ester

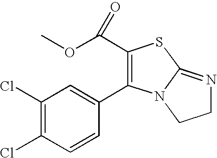

The title compound was prepared from 2-bromo-3-(3,4-dichlorophenyl)-3-oxopropionic acid methyl ester (Preparation 139, 1.40 g, 4.05 mmol) and 2-imidazolidinethione under similar conditions as described in Example 1. δ_{H} (DMSO): 3.70 (3H, s), 4.30 (4H, m), 7.65 (1H, d), 7.90 (1H, m), 8.00 (1H, s).

### Preparation 141

### 2-Bromo-1-(4-chloro-3-trifluoromethylphenyl)ethanone

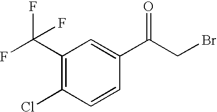

The title compound was prepared from 1-(4-chloro-3-trifluoromethylphenyl)ethanone (5.0 g, 22.46 mmol) under similar conditions as described in Preparation 16. δ_{H} (DMSO): 4.40 (2H, s), 7.70 (1H, d), 8.10 (1H, d), 8.35 (1H, s).

### Preparation 142

### 3-(4-Chloro-3-trifluoromethylphenyl)-5,6-dihydroimidazo[2,1-b]thiazole hydrobromide

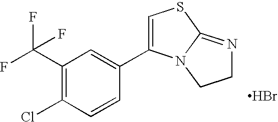

The title compound was prepared from 2-bromo-1-(4-chloro-3-trifluoromethylphenyl)ethanone (Preparation 141, 6.44 g, 21.32 mmol) and 2-imidazolidinethione under similar conditions as described in Example 1. δ_{H} (DMSO): 4.30 (2H, m), 4.55 (2H, m), 7.30 (1H, s), 7.95 (2H, m), 8.10 (1H, s), 9.65 (1H, br s); m/z (ES⁺)=304.93 [M+H]⁺; RT=2.44 min.

### Preparation 143

### 2-Bromo-3-(4-chloro-3-trifluoromethylphenyl)-5,6-dihydroimidazo[2,1-b]thiazole hydrobromide

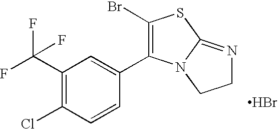

The title compound was prepared from 3-(4-chloro-3-trifluoromethylphenyl-5,6-dihydroimidazo[2,1-b]thiazole hydrobromide (Preparation 142, 5.0 g, 12.99 mmol) and bromine under similar conditions as described in Example 11. δ_{H} (DMSO): 4.35-4.20 (4H, m), 7.95 (1H, d), 8.05 (1H, d), 8.10 (1H, s), 9.60 (1H, br s).

### Preparation 144

### 3-(4-Chloro-3-trifluoromethylphenyl)-5,6-dihydroimidazo[2,1-b]thiazole-2-carbaldehyde

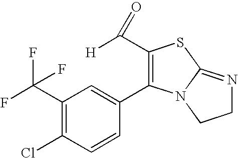

The title compound was prepared from 2-bromo-3-(4-chloro-3-trifluoromethylphenyl)-5,6-dihydroimidazo[2,1-b]thiazole hydrobromide (Preparation 143, 1.5 g, 3.39 mmol) under similar conditions as described in Preparation 138. δ_{H} (DMSO): 3.90 (2H, m), 4.20 (2H, m), 7.95 (1H, m), 8.10 (1H, d), 8.25 (1H, s), 9.20 (1H, s).

### Preparation 145

### 1-(4-Chloro-3-trifluoromethylphenyl)-3-methylbutan-1-one

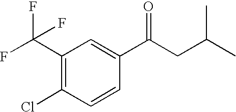

Iso-butyl magnesium bromide 2.0 M solution in diethyl ether (24.0 mL, 48.16 mmol) was slowly added over 5 min to a solution of 4-chloro-3-trifluoromethylbenzonitrile (3.3 g, 16.05 mmol) in THF (50.0 mL) at 0° C. under inert atmosphere. The mixture was stirred for 1 hr before removing the ice bath and stirred for further 16 hr. Saturated ammonium chloride (30 mL) was added slowly to the mixture and then partitioned between EtOAc (150 mL) and saturated sodium chloride (100 mL). The organic layer was dried (MgSO₄) and concentrated in vacuo to afford the title compound. δ_{H} (DMSO): 0.95 (6H, m), 2.15 (1H, m), 3.00 (2H, m), 7.90 (1H, d), 8.30 (2H, m).

### Preparation 146

### 2-Bromo-1-(4-chloro-3-trifluoromethylphenyl)-3-methylbutan-1-one

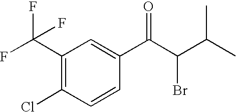

The title compound was prepared from 1-(4-chloro-3-trifluoromethylphenyl)-3-methylbutan-1-one (Preparation 145, 4.0 g, 15.15 mmol) under similar conditions as described in Preparation 16. δ_{H} (DMSO): 1.00 (3H, d), 1.15 (3H, d), 2.35 (1H, m), 5.80 (1H, d), 7.95 (1H, d), 8.40 (2H, m).

### Preparation 147

### 4-Chloro-N-methoxy-3,N dimethylbenzamide

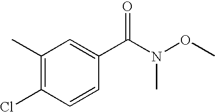

The title compound was prepared from 4-chloro-3-methylbenzoic acid (10.0 g, 58.62 mmol) under similar conditions as described in Preparation 27. δ_{H} (CDCl₃): 2.42 (3H, s), 3.40 (3H, s), 3.60 (3H, s), 7.40 (1H, d), 7.50 (1H, d), 7.60 (1H, s).

### Preparation 148

### 1-(4-Chloro-3-methylphenyl)propan-1-one

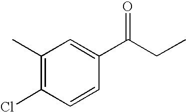

The title compound was prepared from 4-chloro-N-methoxy-3,N-dimethylbenzamide (Preparation 147, 5.0 g, 23.40 mmol) under similar conditions as described in Preparation 28. δ_{H} (DMSO): 1.10 (3H, t), 2.40 (3H, s), 3.05 (2H, q), 7.60 (1H, d), 7.80 (1H, d), 8.00 (1H, s).

### Preparation 149

### 2-Bromo-1-(4-chloro-3-methylphenyl)propan-1-one

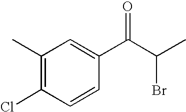

The title compound was prepared from 1-(4-chloro-3-methylphenyl)propan-1-one (4.0 g, 21.90 mmol) under similar conditions as described in Preparation 16. δ_{H} (DMSO): 1.80 (3H, d), 2.42 (3H, s), 5.85 (1H, q), 7.62 (1H, d), 7.90 (1H, d), 8.10 (1H, s).

### Preparation 150

### 2-Bromo-1-(5-fluoronaphthalen-1-yl)propan-1-one

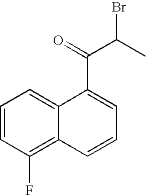

To a stirred solution of 1-(5-fluoronaphthalen-1-yl)propan-1-one (Preparation 183, 0.8 g, 3.96 mmol) in THF (30 mL) was added PTAT (1.49 g, 3.96 mmol) and the reaction was stirred at rt for 72 hr. The reaction mixture was filtered and the filtrate was concentrated in vacuo to afford the title compound. δ_{H} (CDCl₃): 2.00 (3H, d), 5.40 (1H, q), 7.23 (1H, d), 7.60 (2H, m), 7.98 (1H, d), 8.20 (1H, d), 8.40 (1H, d).

### Preparation 151

### 2-Bromo-3-(7-chloronaphthalen-1-yl)-5,6-dihydroimidazo[2,1-b]thiazole hydrobromide

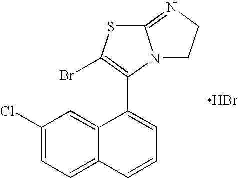

The title compound was prepared from 3-(7-chloronaphthalen-1-yl)-5,6-dihydroimidazo[2,1-b]thiazole hydrobromide (Example 44) under similar conditions as described in Example 11. δ_{H} (DMSO): 3.90 (1H, m), 4.20 (3H, m), 7.71 (1H, d), 7.80 (3H, m), 8.05 (1H, s), 8.17 (1H, d), 8.26 (1H, d), 9.60 (1H, s).

### Preparations 152-157

The following compounds were prepared by the indicated method.

| Prep | Structure | Name | Method | RT min | m/z (ES⁺) |
|---|---|---|---|---|---|
| 152 | 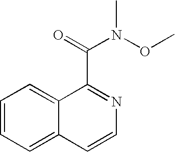 | Isoquinoline-1- carboxylic acid methoxymethyl- amide | Prep 48 | 2.74 | 217.11 [M + H]⁺ |
| 153 | 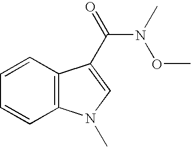 | 1-Methyl-1H- indole-3- carboxylic acidmethoxy- methylamide | Prep 48 | 2.97 | 219.09 [M + H]⁺ |
| 154 | 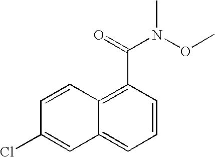 | 6-Chloro- naphthalene-1- carboxylic acid methoxymethyl- amide | Prep 48 | 3.30 | 250.05 [M + H]⁺ |
| 155 | 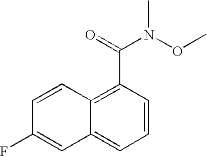 | 6-Fluoro- naphthalene-1- carboxylic acid methoxymethyl- amide | via acid chloride | 3.03 | 234.09 [M + H]⁺ |
| 156 | 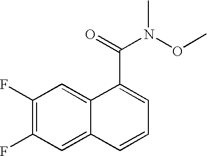 | 6,7-Difluoro- naphthalene-1- carboxylic acid methoxymethyl- amide | via acid chloride | 3.09 | 252.08 [M + H]⁺ |
| 157 | 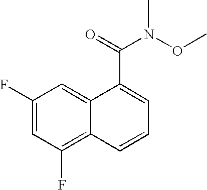 | 5,7-Difluoro- naphthalene-1- carboxylic acid methoxymethyl- amide | via acid chloride | 3.17 | 252.10 [M + H]⁺ |

### Preparations 158-166

The following compounds were prepared by the indicated method

| Prep | Structure | Name | Method | ¹H-NMR data |
|---|---|---|---|---|
| 158 | 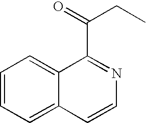 | 1-isoquinolin-1- ylpropan-1-one | Prep 28 | δ_{H}(CD₃OD): 1.23 (3 H, t), 3.29 (2 H, q), 7.68 (1 H, dd), 7.76 (1 H, dd), 7.90-7.96 (2 H, m), 8.51 (1 H, dd), 8.70 (1 H, d) |
| 159 | 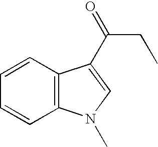 | 1-(1-Methyl-1H- indol-3-yl)- propan-1-one | Prep 28 | δ_{H}(DMSO): 1.14 (3 H, t), 2.86 (2 H, q), 3.88 (3 H, s), 7.24, 7.29 (2 H, 2dd), 7.54 (1 H, d), 8.22 (1 H, d), 8.34 (1 H, s) |
| 160 | 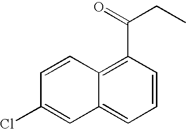 | 1-(6-Chloro- naphthalen-1-yl)- propan-1-one | Prep 28 | δ_{H}(DMSO): 1.16 (3 H, t), 3.14 (2 H, q), 7.63 (1 H, dd), 7.67 (1 H, dd), 8.11-8.16 (3 H, m), 8.50 (1 H, d) |
| 161 | 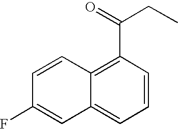 | 1-(6-Fluoro- naphthalen-1-yl)- propan-1-one | Prep 28 | δ_{H}(DMSO): 1.16 (3 H, t), 3.14 (2 H, q), 7.53 (1 H, m), 7.66 (1 H, dd), 7.83 (1 H, dd), 8.07 (1 H, d), 8.13 (1 H, d), 8.54 (1 H, dd) |
| 162 | 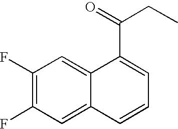 | 1-(6,7-Difluoro- naphthalen-1-yl)- propan-1-one | Prep 28 | δ_{H}(DMSO): 1.08 (3 H, t), 3.09 (2 H, q), 7.59 (1 H, dd), 8.03 (1 H, dd), 8.09 (1 H, d), 8.14 (1 H, d), 8.46 (1 H, dd) |
| 163 | 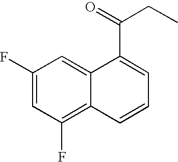 | 1-(5,7-Difluoro- naphthalen-1-yl)- propan-1-one | Prep 28 | δ_{H}(DMSO): 1.16 (3 H, t), 3.17 (2 H, q), 7.59 (1 H, m), 7.72 (1 H, dd), 8.17 (1 H, dd), 8.28 (1 H, d), 8.31 (1 H, d) |
| 164 | 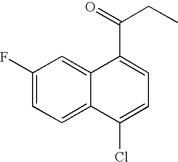 | 1-(4-Chloro-7- fluoro- naphthalen-1-yl)- propan-1-one | Prep 18 | δ_{H}(DMSO): 1.16 (3 H, t), 3.15 (2 H, q), 7.69 (1 H, m), 7.81 (1 H, d), 8.19 (1 H, d), 8.36-8.38 (2 H, m) |
| 165 | 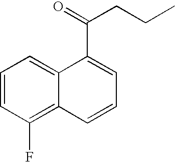 | 1-(5-Fluoro- naphthalen-1-yl)- butan-1-one | Prep 183 | δ_{H}(DMSO): 0.97 (3 H, t), 1.70 (2 H, m), 3.10 (2 H, t), 7.43 (1 H, dd), 7.62 (1 H, m), 7.73 (1 H, dd), 8.15 (1 H, d), 8.24 (1 H, d), 8.28 (1 H, d) |
| 166 | 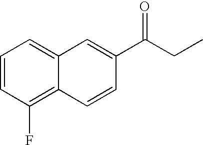 | 1-(5-Fluoro- naphthalen-1-yl)- propan-1-one | Prep 183 | δ_{H}(CDCl₃): 1.33 (3 H, t), 3.17 (2 H, q), 7.30 (1 H, dd), 7.50 (1 H, m), 7.78 (1 H, d), 8.12 (1 H, d), 8.19 (1 H, d), 8.51 (1 H, s) |

### Preparations 167-175

The following compounds were prepared by the indicated methods.

| Prep | Structure | Name | Method | ¹H-NMR data |
|---|---|---|---|---|
| 167 | 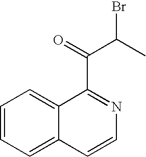 | 2-Bromo-1- isoquinolin-1-yl- propan-1-one | Prep 11 Method A | δ_{H}(DMSO): 1.91 (3 H, d), 6.23 (1 H, q), 7.85 (1 H, dd), 7.91 (1 H, dd), 8.14 (1 H, d), 8.20 (1 H, d), 8.70 (2 H, m) |
| 168 | 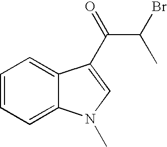 | 2-Bromo-1-(1- methyl-1H-indol- 3-yl)-propan-1- one | Prep 19 | δ_{H}(DMSO): 1.80 (3 H, d), 3.91 (3 H, s), 5.56 (1 H, q), 7.31 (2 H, 2dd), 7.59 (1 H, d), 8.22 (1 H, d), 8.55 (1 H, s) |
| 169 | 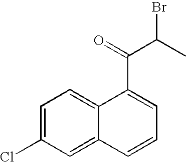 | 2-Bromo-1-(6- chloro- naphthalen-1- yl)propan-1-one | Prep 19 | δ_{H}(DMSO): 1.88 (3 H, d), 5.91 (1 H, q), 7.68-7.73 (2 H, m), 8.18-8.23 (3 H, m), 8.33 (1 H, d) |
| 170 | 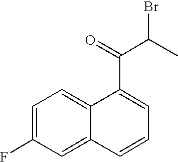 | 2-Bromo-1-(6- fluoro- naphthalen-1- yl)propan-1-one | Prep 19 | δ_{H}(DMSO): 1.89 (3 H, d), 5.91 (1 H, q), 7.59 (1 H, m), 7.67 (1 H, dd), 7.86 (1 H, dd), 8.17-8.19 (2 H, m), 8.38 (1 H, dd) |
| 171 | 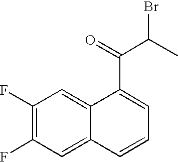 | 2-Bromo-1-(6,7- difluoro- naphthalen-1- yl)propan-1-one | Prep 19 | δ_{H}(DMSO): 1.89 (3 H, d), 5.96 (1 H, q), 7.72 (1 H, dd), 8.17 (1 H, dd), 8.24 (1 H, d), 8.31-8.37 (2 H, m) |
| 172 | 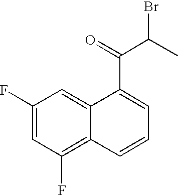 | 2-Bromo-1-(5,7- difluoro- naphthalen-1- yl)propan-1-one | Prep 19 | δ_{H}(DMSO): 1.89 (3 H, d), 5.95 (1 H, q), 7.63 (1 H, m), 7.75 (1 H, dd), 7.97 (1 H, d), 8.33 (1 H, d), 8.40 (1 H, d) |
| 173 | 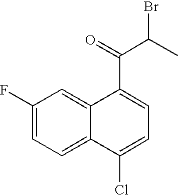 | 2-Bromo-1-(4- chloro-7-fluoro- naphthalen-1- yl)propan-1-one | Prep 19 | δ_{H}(DMSO): 1.88 (3 H, d), 5.92 (1 H, q), 7.75 (1 H, dd), 7.88 (1 H, d), 8.16 (1 H, dd), 8.30 (1 H, d), 8.42 (1 H, dd) |
| 174 | 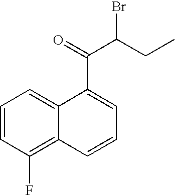 | 2-Bromo-1-(5- fluoro- naphthalen-1- yl)butan-1-one | Prep 19 | δ_{H}(DMSO): 1.10 (3 H, t), 2.07 (2 H, m), 5.75 (1 H, dd), 7.48 (1 H, dd), 7.68 (1 H, m), 7.76 (1 H, dd), 8.13 (1 H, d), 8.28 (1 H, d), 8.34 (1 H, d) |
| 175 | 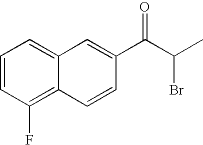 | 2-Bromo-1-(5- fluoro- naphthalen-2- yl)propan-1-one | Prep 19 | δ_{H}(CDCl₃): 2.00 (3 H, d), 5.47 (1 H, q), 7.32 (1 H, dd), 7.53 (1 H, ddd), 7.81 (1 H, d), 8.14 (1 H, dd), 8.22 (1 H, d), 8.60 (1 H, s) |

### Preparation 176

### 2-Bromo-3-(7-chloronaphthalen-1-yl)-5,6-dihydroimidazo[2,1-b]thiazole hydrobromide

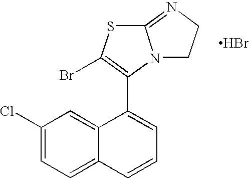

3-(7-Chloronaphthalen-1-yl)-5,6-dihydroimidazo[2,1-b]thiazole hydrobromide (Example 51, 5.30 g, 14.4 mmol) was distributed between saturated NaHCO₃ solution (200 mL) and EtOAc (200 mL). Separation of both layers was followed by further extraction of the aqueous layer with EtOAc (2×100 mL). The combined extracts were washed with brine (100 mL), dried (MgSO₄) and concentrated. The crystalline residue was dissolved in DCM, cooled to 0° C. and treated with bromine (0.75 mL, 14.6 mmol). After removal of the ice bath the thick suspension was stirred for 10 hr at rt. The precipitate (the title compound) was collected and washed with iso-hexane/DCM: 1/1 before being dried in vacuo. δ_{H} (DMSO): 3.93 (1H, m), 4.21-4.26 (3H, m), 7.70 (1H, dd), 7.76 (1H, dd), 7.82 (1H, d), 8.03 (1H, d), 8.17 (1H, d), 8.26 (1H, d), 9.64 (1H, br s); m/z (ES⁺)=365.00, 366.98 [M+H]⁺; RT=2.64 min.

### Preparation 177

### 3-(7-Chloronaphthalen-1-yl)-5,6-dihydroimidazo[2,1-b]thiazole-2-carbaldehyde

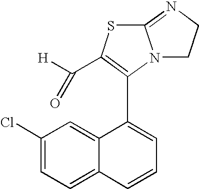

2-Bromo-3-(7-chloronaphthalen-1-yl)-5,6-dihydroimidazo[2,1-b]thiazole hydrobromide (Preparation 176, 4.57 g, 12.4 mmol) in THF (100 mL) at 0° C. After 2 hr DMF (5.6 mL, 64.6 mmol) was added and the mixture was stirred at rt for 12 hr before being distributed between EtOAc (300 mL) and saturated NH₄Cl solution (100 mL). The organic layer was separated, washed (sat NaHCO₃ solution, then brine) and dried (MgSO₄), then concentration in vacuo gave the title compound. δ_{H} (DMSO): 3.57 (2H, m), 4.37 (2H, m), 7.59-7.66 (3H, m), 7.77 (1H, d), 7.96 (1H, d), 8.08 (1H, dd), 9.14 (1H, s); m/z (ES⁺)=315.00 [M+H]⁺; RT=2.49 min.

### Preparation 178

### 6-Fluoronaphthalene-1-carboxylic acid

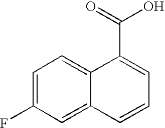

Anhydrous aluminium chloride (65 g, 487 mmol) was added carefully to a suspension of furan-2-carboxylic acid (25 g, 260 mmol) in fluorobenzene (250 mL) at 0° C. After 1 hr the ice bath was removed and the mixture was slowly heated to 75° C. and kept at this temperature for a further 12 hr. The mixture was added to a 2N HCl solution (1.5 L) before extraction into ether (3×300 mL). The combined ether layers were washed with water (250 mL) and then extracted with saturated NaHCO₃ solution (3×250 mL). The alkaline solution was made acidic with conc. HCl solution and re-extracted with EtOAc (3×250 mL). Concentration in vacuo after drying (MgSO₄) gave a solid residue which was stirred in the presence of toluene (50 mL) for 12 hr. Filtration gave the title compound. δ_{H} (DMSO): 7.56 (1H, ddd), 7.66 (1H, dd), 7.84 (1H, dd), 8.16 (2H, m), 8.97 (1H, dd), 13.27 (1H, br s); m/z (ES⁻)=189.19 [M−H]⁺; RT=3.14 min.

### Preparation 179

### 6,7-Difluoronaphthalene-1-carboxylic acid

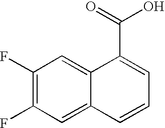

The title compound was prepared from 1,2-difluorobenzene using similar conditions as described in Preparation 178. δ_{H} (DMSO): 2.31 (3H, s), 7.66 (1H, dd), 8.13 (1H, dd), 8.21 (1H, d), 8.27 (1H, d), 8.91 (1H, dd), 13.70 (1H, br s).

### Preparation 180

### 6-Fluoronaphthalen-1-ylamine

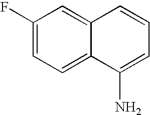

Sodium azide (10.2 g, 157 mmol) was added in small portions over a period of 5 hr to a mixture of 6-fluoronaphthalene-1-carboxylic acid (Preparation 178, 20.0 g, 105 mmol) in CHCl₃ (400 mL) and conc. H₂SO₄ (100 mL) at 40° C. After separation of the CHCl₃ layer the aqueous layer was added onto ice (1 kg). The resulting suspension was made alkaline with conc. NH₄OH solution under cooling and then extracted with Et₂O (3×250 mL). Washing of the combined extracts (brine), drying (MgSO₄) and concentration in vacuo afforded the title compound. δ_{H} (DMSO): 5.82 (2H, br s), 6.66 (1H, d), 7.06 (1H, d), 7.23-7.27 (2H, m), 7.49 (1H, dd), 8.16 (1H, dd); m/z (ES⁺)=162.06 [M+H]⁺; RT=2.57 min.

### Preparation 181

### 1-Chloro-6-fluoronaphthalene

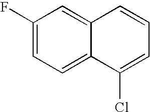

A solution of sodium nitrite (2.16 g, 31.3 mmol) in water (50 mL) was added over 15 min to a suspension of 6-fluoronaphthalen-1-ylamine (Preparation 180, 5.50 g, 34.2 mmol) in dilute HCl (100 mL, 6M) at 0° C. The resulting mixture was stirred for 1 hr in the cold and then added to a suspension of copper(I)chloride in dilute HCl (100 mL, 6M). After 2 hr of vigorous stirring water (1 L) was added to the suspension which was extracted with ether (3×200 mL). Drying of the combined extracts (MgSO₄) and subsequent concentration in vacuo gave a residue which was purified by flash-chromatography on silica gel (eluent: hexane/EtOAc: 6/1) to give the title compound. δ_{H} (DMSO): 7.33 (1H, dd), 7.38 (1H, ddd), 7.46 (1H, d), 7.63 (1H, dd), 7.72 (1H, d), 8.01 (1H, dd).

### Preparation 182

### 1-(5-Fluoronaphthalen-1-yl)propan-1-ol

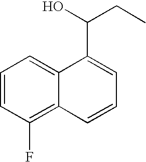

n-Butyllithium (5.5 mL, 2.5 M solution in hexane) was added slowly to a solution of 1-bromo-5-fluoronaphthalene (M. S. Newman et al, J. Org. Chem., 1959, 24, 509-512) (2.40 g, 10.7 mmol) in THF (50 mL) at −78° C. After stirring for 1 hr propionaldehyde (2.5 mL, 34.7 mmol) was added to the green solution and the CO₂/IPA bath was removed. After stirring for a further 90 min the mixture was added to sat. NH₄Cl solution (250 mL). Extraction with EtOAc (3×100 mL), drying of the combined extracts (MgSO₄) and subsequent concentration in vacuo gave a residue which was purified by flash-chromatography on silica gel (eluent: hexane/EtOAc: 4/1) to give the title compound. δ_{H} (DMSO): 0.93 (3H, t), 1.72 (2H, m), 5.24 (1H, m), 5.37 (1H, d), 7.33 (1H, dd), 7.53 (1H, m), 7.64 (1H, dd), 7.74 (1H, d), 7.98 (1H, d), 8.01 (1H, d).

### Preparation 183

### 1-(5-Fluoronaphthalen-1-yl)propan-1-one

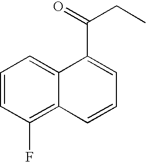

To a solution of 1-(5-fluoronaphthalen-1-yl)propan-1-ol (Preparation 182, 1.62 g, 7.93 mmol) in dry DCM (75 mL) was added Dess-Martin periodinane (3.40 g, 8.02 mmol). After stirring for 2 hr at rt alkaline sodium thiosulfate solution was added (8.0 g Na₂SO₃ dissolved in 30 mL saturated NaHCO₃ solution) and the emulsion was vigorously stirred for an additional 10 min before further diluted with water (˜100 mL). Extraction with EtOAc (3×50 mL), washing of the combined extracts with saturated sodium hydrogen carbonate (50 mL) and brine (50 mL). After drying (MgSO₄) and concentration in vacuo the residue was purified by flash-chromatography on silica gel (eluent: hexane/EtOAc: 4/1) to give the title compound. δ_{H} (DMSO): 1.17 (3H, t), 3.14 (2H, q), 7.43 (1H, dd), 7.61 (1H, m), 7.73 (1H, dd), 8.16 (1H, d), 8.26 (2H, m).

### Preparation 184

### 1-(5-Fluoronaphthalen-1-yl)butan-1-ol

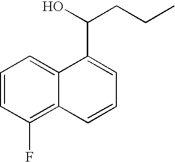

The title compound was prepared from 1-bromo-5-fluoronaphthalene and butyraldehyde under similar conditions as described in Preparation 182. δ_{H} (DMSO): 0.91 (3H, t), 1.44, 1.55 (4H, 2m), 5.30 (1H, m), 5.37 (1H, d), 7.33 (111, dd), 7.54 (1H, m), 7.62 (1H, dd), 7.75 (1H, d), 7.98 (1H, d), 8.01 (1H, d).

### Preparation 185

### 1-Fluoro-6-methylnaphthalene

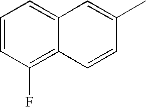

A solution of sodium nitrite (1.84 g, 26.7 mmol) in water (20 mL) was added in portions under vigorous sting to a suspension of 6-methylnaphthalen-1-ylamine (Preparation 79, 4.10 g, 26.1 mmol) in dilute HCl (40 mL, 6M) at 0° C. After 2 hr tetrafluoroboric acid (9.5 mL, 48%) was added and the mixture was stirred for a further 30 min in the cold. The precipitate was collected, washed with aqueous tetrafluoroboric acid (50 mL, 20%) and water (50 mL) and dried in vacuo at rt for 3 days. The powdered diazonium salt was heated to 160° C. for 15 min. After cooling to rt the residue was taken up in ether (300 mL) and washed with NaHCO₃ solution and brine. Drying (MgSO₄) and concentration in vacuo gave a residue which was purified by flash-chromatography on silica gel (eluent: hexane/EtOAc: 9/1) to give the title compound. Diazonium salt: δ_{H} (DMSO): 2.62 (3H, s), 7.98-8.05 (2H, m), 8.22 (1H, s), 8.43 (1H, d), 8.83 (1H, d), 9.13 (1H, d); Title compound. δ_{H} (DMSO): 2.51 (3H, s), 7.25 (1H, dd), 7.45-7.50 (1H, m), 7.69 (1H, d), 7.79 (1H, s), 7.97 (1H, d).

### Preparation 186

### 5-Fluoronaphthalene-2-carbaldehyde

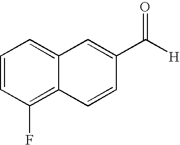

A mixture of selenium dioxide (2.07 g, 18.7 mmol) and 1-fluoro-6-methylnaphthalene (Preparation 185, 734 mg, 4.58 mmol) in dioxane (20 mL) was heated under reflux for 7 days. The mixture was diluted with EtOAc (200 mL), filtered and washed with water (50 mL) and brine (50 mL). Drying (MgSO₄) and concentration in vacuo gave a residue which was purified by flash-chromatography on silica gel (eluent: hexane/EtOAc: 4/1) to give the title compound. δ_{H} (CDCl₃): 7.27 (1H, dd), 7.46 (1H, ddd), 7.74 (1H, d), 7.93 (1H, dd), 8.13 (1H, d), 8.29 (1H, s), 10.11 (1H, s).

### Preparation 187

### 1-(5-Fluoronaphthalen-2-yl)propan-1-ol

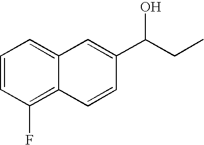

Ethyl magnesium chloride (0.8 mL, 2 M solution in ether) was added to a solution of 5-fluoronaphthalene-2-carbaldehyde (Preparation 186, 117 mg, 0.672 mmol) in THF at −78° C. The CO₂/IPA bath was removed and the mixture was stirred for 12 hr at rt before sat. NH₄Cl solution (50 mL) was added. The layers were separated and the aqueous layer was extracted with EtOAc (3×30 mL). The combined organic layers were washed (brine), dried (MgSO₄) and concentrated in vacuo. Purification by flash-chromatography on silica gel (eluent: hexane/EtOAc: 4/1) gave the title compound. δ_{H} (CDCl₃): 0.99 (3H, t), 1.91 (2H, dq), 4.82 (1H, t), 7.16 (1H, dd), 7.43 (1H, ddd), 7.56 (1H, dd), 7.65 (1H, d), 7.84 (1H, s), 8.13 (1H, d).

### Example 1

### 3-Naphthalen-1-yl-5,6-dihydroimidazo[2,1-b]thiazole hydrobromide

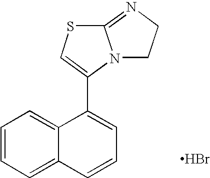

2-Bromo-1-naphthalen-1-yl-ethanone (Preparation 1, 0.643 g, 2.58 mmol) and 2-imidazolidinethione (0.264 g, 2.58 mmol) were dissolved in EtOH (15 mL) and AcOH (7 mL) and the reaction heated under reflux for 16 hr. The reaction mixture was cooled to rt and the precipitate filtered, washed with Et₂O (2×20 mL) and dried to afford the title compound. δ_{H} (DMSO): 4.06 (2H, m), 4.26 (2H, m), 7.03 (1H, s), 7.62-7.70 (4H, m), 7.98 (1H, m), 8.07 (1H, m), 8.14 (1H, m), 9.66 (1H, br s); m/z (ES⁺)=253 [M+H]⁺; RT=2.44 min.

### Example 2

### 3-Naphthalen-2-yl-5,6-dihydroimidazo[2,1-b]thiazole hydrobromide

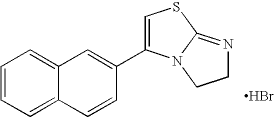

2-Bromo-1-naphthalen-2-ylethanone (0.25 g, 1 mmol) and 2-imidazolidinethione (0.1 g, 1 mmol) were dissolved in EtOH (8 mL) and AcOH (4 mL) and the reaction heated under reflux for 4 hr. The reaction mixture was then cooled to rt and the precipitate was filtered, washed with Et₂O (2×20 mL) and dried in vacuo to afford the title compound. δ_{H} (DMSO): 4.06 (2H, m), 4.26 (2H, m), 7.03 (1H, s), 7.62-7.70 (4H, m), 7.98 (1H, m), 8.07 (1H, m), 8.14 (1H, m), 9.66 (1H, br s); m/z (ES⁺)=253 [M+H]⁺; RT=2.44 min.

### Example 3

### 2-Methyl-3-naphthalen-2-yl-5,6-dihydroimidazo[2,1-b]thiazole hydrobromide

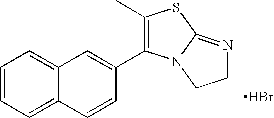

2-Bromo-1-naphthalen-2-ylpropan-1-one (Preparation 3, 3.74 g, 14.2 mmol) and 2-imidazolidinethione (1.45 g, 14.2 mmol) were dissolved in AcOH (20 mL) and EtOH (40 mL) and heated to reflux for 16 hr. The reaction mixture was cooled to rt and the precipitated solid was filtered, washed with Et₂O (2×40 mL) and dried to afford the title compound. δ_{H} (DMSO): 2.31 (3H, s), 4.25 (2H, m), 4.37 (2H, m), 7.64 (3H, m), 8.04 (2H, m), 8.11-8.15 (2H, m), 9.60 (1H, br s); m/z (ES⁺)=267.1 [M+H]⁺; RT=2.42 min.

### Example 4

### 2-Bromo-3-naphthalen-2-yl-5,6-dihydroimidazo[2,1-b]thiazole hydrobromide

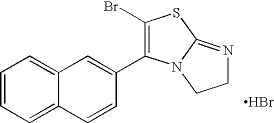

3-Naphthalen-2-yl-5,6-dihydroimidazo[2,1-b]thiazole hydrobromide (Example 2, 1 g, 3.0 mmol) was partitioned between saturated Na₂CO₃ solution (30 mL) and DCM (3×30 mL). The combined organic fractions were dried (MgSO₄) and concentrated in vacuo. The residue was dissolved in DCM (10 mL) and cooled to 0° C. Bromine (154 μL, 3.0 mmol) was added and the reaction stirred at 0° C. for 30 min then warmed to rt and stirred for 1 hr. The reaction mixture was diluted with Et₂O (30 mL) and the solid filtered and washed with Et₂O (2×30 mL) to afford the title compound. δ_{H} (DMSO): 4.25 (2H, m), 4.38 (2H, m), 7.66 (3H, m), 8.04 (1H, d), 8.08 (1H, d), 8.14 (1H, d), 8.21 (1H, s), 9.62 (1H, br s); m/z (ES⁺)=331 [M+H]⁺; RT=2.86 min.

### Example 5

### 2-Chloro-3-naphthalen-1-yl-5,6-dihydroimidazo[2,1-b]thiazole hydrochloride

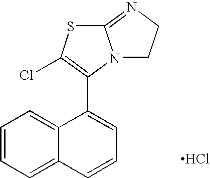

3-Naphthalen-1-yl-5,6-dihydroimidazo[2,1-b]thiazole hydrobromide (Example 1, 1 g, 3.0 mmol) was partitioned between saturated NaHCO₃ solution (40 mL) and DCM (2×50 mL). The combined organic fractions were dried (MgSO₄) and concentrated in vacuo. The residue was dissolved in acetone (70 mL) and cooled to 0° C. Benzyltrimethylammonium tetrachloroiodate (1.28 g, 3.1 mmol) was added portionwise over 5 min and the reaction stirred at 0° C. for 1 hr, then rt for 2 hr. The reaction mixture was filtered and washed with MeCN:Et₂O (1:2, 2×25 mL), then Et₂O (2×25 mL). The solid was triturated with hot ⁱPrOH to afford the title compound. δ_{H} (DMSO): 3.91 (1H, m), 4.19 (3H, m), 7.71 (4H, m), 7.91 (1H, m), 8.10 (1H, m), 8.20 (1H, m), 10.42 (1H, br s); m/z (ES⁺)=286 [M+H]⁺; RT=2.56 min.

### Example 6

### 3-Thieno[2,3-b]thiophen-2-yl-5,6-dihydroimidazo[2,1-b]thiazole hydrobromide

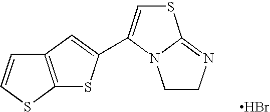

2-Bromo-1-thieno[2,3-b]thiophen-2-ylethanone (Preparation 6, 420 mg, 1.61 mmol) and 2-imidazolidinethione (164 mg, 1.61 mmol) were heated to reflux in AcOH (5 mL) and EtOH (10 mL) for 48 hr. The reaction mixture was cooled to rt and the precipitated solid was filtered and washed with Et₂O to afford the title compound. δ_{H} (DMSO): 4.34 (2H, m), 4.64 (2H, m), 7.05 (1H, s), 7.38 (1H, d), 7.72 (1H, d), 7.75 (1H, s), 9.75 (1H, br s); m/z (ES⁺)=265 [M+H]⁺; RT=2.54 min.

### Example 7

### 3-(4-Methylnaphthalen-1-yl)-5,6-dihydroimidazo[2,1-b]thiazole hydrobromide

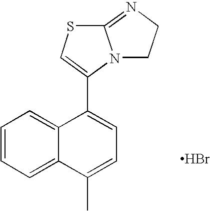

A stirred solution of 2-bromo-1-(4-methylnaphthalen-1-yl)ethanone (Preparation 7, 0.54 g, 1.9 mmol) and imidazolidine-2-thione (0.2 g, 1.9 mmol) in EtOH (10 mL) was heated to reflux and AcOH (5 mL) was added. The reaction was stirred at reflux for 24 hr then cooled to 0° C. The solid was collected by filtration to afford the title compound. δ_{H} (DMSO): 2.73 (3H, s), 4.01 (2H, m), 4.24 (2H, m), 6.94 (1H, s), 7.51 (1H, d), 7.57 (1H, d), 7.67 (2H, m), 7.97 (1H, d), 8.16 (1H, d), 9.59 (1H, br s); m/z (ES⁺)=267 [M+H]⁺; RT=2.90 min.

### Example 8

### 2-(5,6-Dihydroimidazo[2,1-b]thiazol-3-yl)quinoline hydrobromide

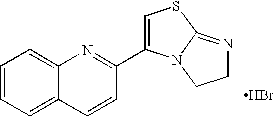

To a solution of 2-bromo-1-quinolin-2-ylethanone (Preparation 11, 98 mg, 0.392 mmol) in a mixture of EtOH and AcOH (2:1, 15 mL) was added imidazolidine-2-thione (42 mg, 0.411 mmol) and the resulting suspension heated under reflux for 12 hr. On cooling in an ice bath a precipitate formed spontaneously, which was collected and washed with EtOAc to give the title compound. δ_{H} (DMSO): 4.39 (2H, dd), 5.07 (2H, dd), 7.72 (1H, m), 7.83 (1H, s), 7.88 (1H, m), 8.08 (2H, m), 8.16 (1H, d), 8.55 (1H, d), 9.69 (1H, br s); m/z (ES⁺)=254.09 [M+H]⁺; RT=2.64 min.

### Example 9

### 3-(4-Fluoronaphthalen-1-yl)-5,6-dihydroimidazo[2,1-b]thiazole hydrobromide

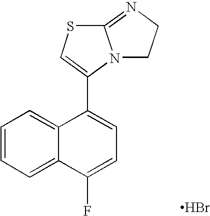

2-Bromo-1-(4-fluoronaphthalen-1-yl)ethanone (Preparation 12, 0.60 g, 2.25 mmol) and 2-imidazolidinethione (0.23 g, 2.25 mmol) were heated to reflux in AcOH (7 mL) and EtOH (14 mL) for 16 hr. The reaction mixture was cooled to rt and the precipitated solid was filtered and washed with Et₂O to afford the title compound. δ_{H} (DMSO): 4.06 (2H, m), 4.27 (2H, m), 7.04 (1H, s), 7.52 (1H, m), 7.75 (3H, m), 8.05 (1H, m), 8.19 (1H, m), 9.68 (1H, br s); m/z (ES⁺)=271 [M+H]⁺; RT=1.77 min.

### Example 10

### 3-Naphthalen-2-yl-5,6-dihydroimidazo[2,1-b]thiazole-2-carboxylic acid ethyl ester hydrobromide

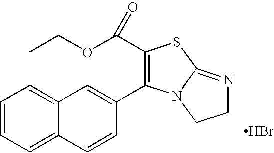

2-Bromo-3-naphthalen-2-yl-3-oxopropionic acid ethyl ester (Preparation 14, 5.58 g, 17.4 mmol) and 2-imidazolidinethione (1.78 g, 17.4 mmol) were heated to reflux in AcOH (25 mL) and EtOH (50 mL) for 16 hr. The solvent was removed in vacuo and the residue dissolved in MeCN (80 mL) and Et₂O (5 mL). The precipitated solid was filtered and washed with Et₂O (2×40 mL) to afford the title compound. δ_{H} (DMSO): 1.03 (3H, t), 4.12 (2H, q), 4.29 (4H, m), 7.66 (3H, m), 8.04 (2H, d), 8.08 (1H, d), 8.24 (1H, s), 10.10 (1H, br s); m/z (ES⁺)=325 [M+H]⁺; RT=2.69 min.

### Example 11

### 2-Bromo-3-naphthalen-1-yl-5,6-dihydroimidazo[2,1-b]thiazole hydrobromide

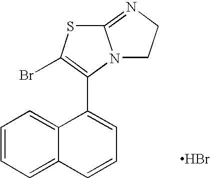

3-Naphthalen-1-yl-5,6-dihydroimidazo[2,1-b]thiazole hydrobromide (Example 1, 5 g, 15.0 mmol) was partitioned between saturated NaHCO₃ solution (70 mL) and DCM (2×75 mL). The combined organic fractions were dried MgSO₄) and concentrated in vacuo. The residue was dissolved in DCM (50 mL) and cooled to 0° C. A solution of bromine (0.77 mL, 15.0 mmol) in DCM (7 mL) was added dropwise, the reaction warmed to rt and stirred for 3 hr. The reaction mixture was diluted with Et₂O (50 mL) and the precipitate was filtered, washed with MeCN:Et₂O (2:1, 3×50 mL) and then Et₂O (2×30 mL) to afford the title compound. δ_{H} (DMSO): 3.90 (1H, m), 4.17 (3H, m), 7.66 (2H, m), 7.72 (2H, m), 7.89 (1H, m), 8.10 (1H, m), 8.20 (1H, d), 9.66 (1H, br s); m/z (ES⁺)=331 [M+H]⁺; RT=2.44 min.

### Example 12

### 2-Methyl-3-naphthalen-1-yl-5,6-dihydroimidazo[2,1-b]thiazole hydrobromide

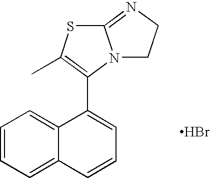

2-Bromo-1-naphthalen-1-ylpropanone (Preparation 16, 4.28 g, 16.3 mmol) and imidazolidine-2-thione (1.64 g, 16.3 mmol) were dissolved in EtOH (15 mL) and AcOH (7.5 mL) and the reaction heated to reflux for 4 hr. The reaction mixture was cooled overnight and the precipitate collected by filtration, washed with acetonitrile (20 mL) and Et₂O (2×20 mL) then dried to afford the title compound. δ_{H} (DMSO): 2.05 (3H, s), 3.82 (1H, m), 4.07 (1H, m), 4.28 (2H, m), 7.65-7.70 (4H, m), 7.81 (1H, m), 8.05 (1H, m), 8.18 (1H, m), 9.58 (1H, br s); m/z (ES⁺)=266.99 [M+H]⁺; RT=2.62 min.

### Example 13

### 3-Naphthalen-1-yl-5,6-dihydroimidazo[2,1-b]thiazol-2-yl methanol

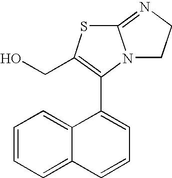

3-Naphthalen-1-yl-5,6-dihydroimidazo[2,1-b]thiazole-2-carbaldehyde (Preparation 17, 281 mg, 1.0 mmol) was suspended in MeOH (10 mL) under an argon atmosphere and cooled to 0° C. Sodium borohydride (57 mg, 1.5 mmol) was added and the reaction stirred at 0° C. for 2 hr, then rt for 16 hr. Water (40 mL) was added and the precipitate was filtered, washed with water (2×20 mL) and dried under vacuum at 40° C. to afford the title compound. δ_{H} (DMSO): 3.32 (2H, s), 3.98 (4H, m), 5.19 (1H, br s), 7.59 (4H, m), 7.85 (1H, m), 8.04 (2H, m); m/z (ES⁺)=283 [M+H]⁺; RT=2.26 min.

### Example 14

### 2-Ethynyl-3-naphthalen-1-yl-5,6-dihydroimidazo[2,1-b]thiazole

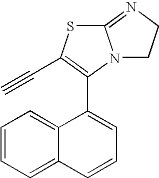

N-Butyllithium (0.256 mL, 0.64 mmol) was added to diisopropylamine (89.4 μL, 0.64 mmol) in THF at 0° C. After 10 min the solution was cooled to −78° C. and trimethylsilyldiazomethane (0.51 mL, 1.02 mmol, 2M in THF) was added. After 1 hr 3-naphthalen-1-yl-5,6-dihydroimidazo[2,1-b]thiazole-2-carbaldehyde (Preparation 17, 150 mg, 0.535 mmol) in THF (10 mL) was added dropwise and the reaction allowed to warm to rt. After heating under reflux for 3 hr the solution was allowed to cool and partitioned between DCM (3×25 mL) and water (20 mL). The organic layer was separated, dried (MgSO₄), filtered and concentrated in vacuo. The residue was purified on silica gel by elution with acetone:hexane (1:1) to afford the title compound. δ_{H} (CDCl₃) 7.85 (3H, m), 7.42 (4H, m), 4.10 (2H, m), 3.40 (2H, m), 2.90 (1H, s); m/z (ES⁺)=277.01 [M−H]⁺; RT=2.45 min.

### Example 15

### 3-(7-chloronaphthalen-1-yl)-2-methyl-5,6-dihydroimidazo[2,1-b]thiazole hydrobromide

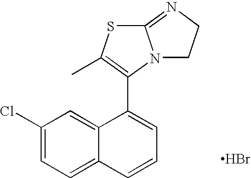

To a solution of 2-bromo-1-(7-chloronaphthalen-1-yl)propan-1-one (Preparation 19, 1.45 g, 4.87 mmol) in a mixture of EtOH and AcOH (1:1, 100 mL) was added imidazolidine-2-thione (580 mg, 5.68 mmol) and the resulting suspension heated under reflux for 12 hr. The solvent was removed in vacuo and the residue distributed between dilute NaOH solution (300 mL) and EtOAc (100 mL). Separation of both layers was followed by further extraction of the aqueous layer with EtOAc (2×100 mL). The combined extracts were washed with brine (100 mL), dried (MgSO₄) and concentrated. Purification by flash-chromatography on silica gel (eluent DCM:MeOH, 10:1) gave the free base of the title compound. The free base was taken up in MeOH (150 mL) and treated with hydrobromic acid (30% in acetic acid, 1.0 mL). After removal of the solvent the residue was stirred in a mixture of EtOAc and acetonitrile (10:1, 50 mL) until the title compound crystallised out and was collected by filtration. δ_{H} (DMSO): 2.06 (3H, s), 3.89 (1H, ddd), 4.06-4.27 (3H, m), 7.69 (1H, dd), 7.72-7.78 (2H, m), 7.94 (1H, s), 8.16 (1H, d), 8.23 (1H, dd), 9.55 (1H, br s); m/z (ES⁺)=300.96 [M+H]⁺; RT=2.56 min.

### Example 16

### 1-(3-Naphthalen-1-yl-5,6-dihydroimidazo[2,1-b]thiazol-2-yl)ethanol

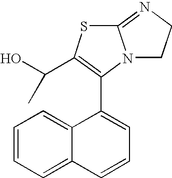

3-Naphthalen-1-yl-5,6-dihydroimidazo[2,1-b]thiazole-2-carbaldehyde (Preparation 17, 200 mg, 0.7 mmol) was dissolved in THF (25 mL) under an argon atmosphere and cooled to 0° C. Methylmagnesium bromide (3.0M in Et₂O, 0.71 mL, 2.14 mmol) was added and the reaction stirred at 0° C. for 1 hr, then rt for 16 hr. The reaction was quenched with water (40 mL) and extracted into EtOAc (3×40 mL). The combined organic fractions were dried (MgSO₄) and concentrated in vacuo to afford the title compound. δ_{H} (CDCl₃): 1.24, 1.35 (3H, 2d), 2.52 (1H, br s), 3.30 (2H, m), 4.03 (2H, m), 4.49 (1H, m), 7.27-7.53 (4H, m), 7.73-7.90 (3H, m); m/z (ES⁺)=297 [M+H]⁺; RT=2.26 min.

### Example 17

### 3-Naphthalen-1-yl-5,6-dihydroimidazo[2,1-b]thiazole-2-Carbonitrile

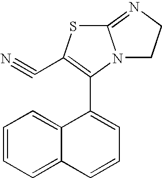

3-Naphthalen-1-yl-5,6-dihydroimidazo[2,1-b]thiazole-2-carbaldehyde (Preparation 17, 300 mg, 1.07 mmol) and hydroxylamine hydrochloride (97 mg, 1.39 mmol) were dissolved in formic acid (10 mL) and heated to 100° C. for 16 hr. The reaction mixture was diluted with Et₂O (50 mL) and the solid filtered and washed with Et₂O (2×30 mL). The solid was purified by chromatography on silica gel eluting with MeOH:DCM (3:97) to afford the title compound. δ_{H} (CDCl₃): 3.50 (1H, m), 3.61 (1H, m), 4.28 (2H, m), 7.62 (4H, m), 7.79 (1H, d), 7.97 (1H, d), 8.04 (1H, d); m/z (ES⁺)=278 [M+H]⁺; RT=2.31 min.

### Example 18

### 2-Methylsulfanyl-3-naphthalen-1-yl-5,6-dihydroimidazo[2,1-b]thiazole

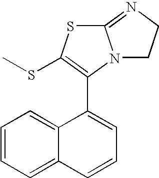

2-Bromo-3-naphthalen-1-yl-5,6-dihydroimidazo[2,1-b]thiazole hydrobromide (Example 11, 0.5 g, 1.21 mmol) was suspended in THF (20 mL) under an argon atmosphere and cooled to 0° C. Ethylmagnesium chloride (2.0M in Et₂O, 1.8 mL, 3.64 mmol) was added dropwise over 5 min and the reaction stirred at 0° C. for 2 hr. Dimethyl disulphide (0.22 mL, 2.43 mmol) was added and the reaction mixture stirred at rt for 16 hr. The reaction was quenched with saturated NH₄Cl solution (40 mL) then extracted into EtOAc (3×30 mL). The combined organic fractions were dried (MgSO₄), concentrated in vacuo and purified by chromatography on silica gel eluting with MeOH:DCM (1:19) to afford the title compound. δ_{H} (CDCl₃): 2.18 (3H, s), 3.41 (2H, m), 4.10 (2H, m), 7.45 (1H, d), 7.55 (3H, m), 7.83 (1H, m), 7.95 (2H, m); m/z (ES⁺)=299 [M+H]⁺; RT=2.45 min.

### Example 19

### (3-Naphthalen-2-yl-5,6-dihydroimidazo[2,1-b]thiazol-2-yl)methanol

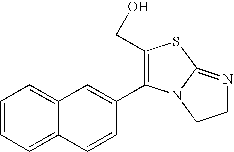

3-Naphthalen-2-yl-5,6-dihydroimidazo[2,1-b]thiazole-2-carbaldehyde (Preparation 20, 0.5 g, 1.78 mmol) was suspended in MeOH (20 mL) under an argon atmosphere and cooled to 0° C. Sodium borohydride (0.10 g, 2.68 mmol) was added and the reaction stirred at 0° C. for 0.5 hr, then warmed to rt for 2 hr. Water (40 mL) was added and the precipitate was filtered and washed with water (2×20 mL), and Et₂O (2×20 mL) and dried to afford the title compound. δ_{H} (DMSO): 3.70 (2H, m), 4.01 (2H, m), 4.28 (2H, d), 5.34 (1H, m), 7.58 (3H, m), 7.98 (3H, m), 8.03 (1H, m); m/z (ES⁺)=283 [M+H]⁺; RT=2.27 min.

### Example 20

### 1-(3-Naphthalen-2-yl-5,6-dihydroimidazo[2,1-b]thiazol-2-yl)ethanol

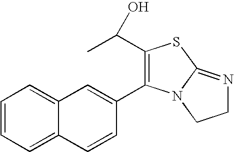

3-Naphthalen-2-yl-5,6-dihydroimidazo[2,1-b]thiazole-2-carbaldehyde (Preparation 20, 200 mg, 0.71 mmol) was dissolved in THF (25 mL) under an argon atmosphere and cooled to 0° C. Methylmagnesium bromide (3.0M in Et₂O, 0.71 mL, 2.14 mmol) was added dropwise and the reaction maintained at 0° C. for 1 hr, then stirred at rt for 16 hr. Water (40 mL) was added and the reaction mixture extracted into EtOAc (3×30 mL). The combined organic fractions were dried (MgSO₄) and concentrated in vacuo to afford the title compound. δ_{H} (CDCl₃): 1.46 (3H, d), 3.40 (1H, br s), 3.66 (2H, m), 4.14 (2H, m), 4.86 (1H, m), 7.47 (1H, d), 7.56 (2H, m), 7.90 (4H, m); m/z (ES⁺)=297 [M+H]⁺; RT=2.40 min.

### Example 21

### 2-(3-Naphthalen-2-yl-5,6-dihydroimidazo[2,1-b]thiazol-2-yl)propan-2-ol

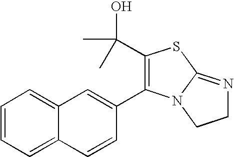

3-Naphthalen-2-yl-5,6-dihydroimidazo[2,1-b]thiazole-2-carboxylic acid ethyl ester hydrobromide (Example 10, 1 g, 2.5 mmol) was partitioned between saturated NaHCO₃ solution (40 mL) and DCM (3×30 mL). The combined organic fractions were dried (MgSO₄) and concentrated in vacuo. The residue was dissolved in THF (20 mL) under an argon atmosphere and cooled to 0° C. Methylmagnesium bromide (3.0M in Et₂O, 2.5 mL, 7.4 mmol) was added dropwise and the reaction stirred at 0° C. for 1 hr, then rt for 16 hr. Water (40 mL) was added and the reaction mixture was extracted into EtOAc (3×30 mL). The combined organic fractions were dried (MgSO₄) and concentrated in vacuo to afford the title compound. δ_{H} (CDCl₃): 1.37 (6H, s), 3.43 (2H, m), 4.06 (2H, m), 7.44 (1H, dd), 7.56 (2H, m), 7.89 (4H, m); m/z (ES⁺)=311 [M+H]⁺; RT=2.45 min.

### Example 22

### 3-(4-Fluoronaphthalen-1-yl)-2-methyl-5,6-dihydroimidazo[2,1-b]thiazole hydrobromide

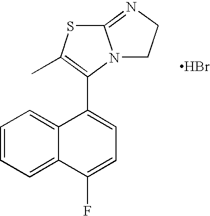

2-Bromo-1-(4-fluoronaphthalen-1-yl)propan-1-one (Preparation 22, 4.59 g, 16.3 mmol) and 2-imidazolidinethione (1.67 g, 16.3 mmol) were heated to reflux in AcOH (20 mL) and EtOH (40 mL) for 24 hr. The solvent was removed in vacuo and the residue dissolved in MeCN (10 mL), followed by addition of Et₂O (80 mL). The precipitated solid was filtered and washed with Et₂O (2×30 mL) to afford the title compound. δ_{H} (DMSO): 2.05 (3H, s), 3.85 (1H, m), 4.09 (1H, m), 4.20 (2H, m), 7.54 (1H, m), 7.71 (1H, m), 7.75 (2H, m), 7.88 (1H, m), 8.20 (1H, m), 9.56 (1H, br s); m/z (ES⁺)=285 [M+H]⁺; RT=2.56 min.

### Example 23

### 2-Ethyl-3-naphthalen-1-yl-5,6-dihydroimidazo[2,1-b]thiazole hydrobromide

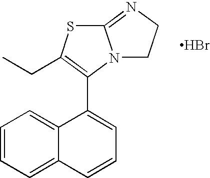

2-Bromo-1-naphthalen-1-ylbutan-1-one (Preparation 24, 0.82 g, 3.0 mmol) and 2-imidazolidinethione (0.30 g, 3.0 mmol) was dissolved in AcOH (10 mL) and EtOH (20 mL) and heated to reflux for 8 hr, then stirred at rt for 72 hr. The solvent was removed in vacuo and the residue partitioned between saturated NaHCO₃ solution (40 mL) and EtOAc (2×40 mL). The combined organic fractions were dried (MgSO₄), concentrated in vacuo and purified by chromatography on silica gel eluting with MeOH:DCM (2:23). The residue was dissolved in EtOAc (20 mL) and 30% HBr in AcOH (1 mL) was added. The solvent was removed in vacuo to afford the title compound. δ_{H} (DMSO): 1.04 (3H, t), 2.33-2.47 (2H, m), 3.83 (1H, m), 4.06 (1H, m), 4.21 (2H, m), 7.67 (4H, m), 7.83 (1H, m), 8.09 (1H, m), 8.17 (1H, m), 9.68 (1H, br s); m/z (ES⁺)=281 [M+H]⁺; RT=2.52 min.

### Example 24

### 2-Isopropyl-3-naphthalen-1-yl-5,6-dihydroimidazo[2,1-b]thiazole hydrobromide

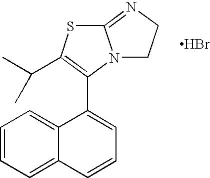

2-Bromo-3-methyl-1-naphthalen-1-ylbutan-1-one (Preparation 26, 0.39 g, 1.3 mmol) and 2-imidazolidinethione (0.14 g, 1.3 mmol) were dissolved in AcOH (5 mL) and EtOH (10 mL) and heated to reflux for 16 hr. The solvent was removed in vacuo and the residue partitioned between saturated NaHCO₃ solution (40 mL) and EtOAc (3×30 mL). The combined organic fractions were dried (MgSO₄), concentrated in vacuo and purified by chromatography on silica gel eluting with MeOH:DCM (2:23). The residue was dissolved in MeCN (20 mL) followed by addition of 30% HBr in AcOH (0.5 mL). The solvent was removed in vacuo and the residue dissolved in MeCN (5 mL) and added to Et₂O (25 mL). The solid was filtered and dried to afford the title compound. δ_{H} (DMSO): 1.10 (3H, d), 1.15 (3H, d), 2.69 (1H, m), 3.80 (1H, m), 4.01 (1H, m), 420 (2H, m), 7.68 (4H, m), 7.84 (1H, m), 8.09 (1H, m), 8.17 (1H, m), 9.74 (1H, br s); m/z (ES⁺)=295 [M+H]⁺; RT=2.62 min.

### Example 25

### [3-(7-Chloronaphthalen-1-yl)-5,6-dihydroimidazo[2,1-b]thiazol-2-yl]-methanol

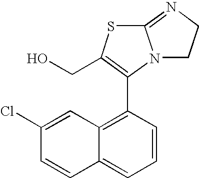

To a solution of 3-(7-chloronaphthalen-1-yl)-5,6-dihydroimidazo[2,1-b]thiazole-2-carbaldehyde (Preparation 177, 1.14 g, 3.62 mmol) in ethanol/water: 5/1 (60 mL) was added sodium borohydride (300 mg, 7.92 mmol). After stirring for 12 hr the solution was acidified with dilute HCl solution and further diluted with water (100 mL). After concentration in vacuo the residue was twice codestillated with methanol (˜50 mL) before being distributed between EtOAc (200 mL) and sat. NaHCO₃ solution (200 mL). The organic layer was separated and the aqueous layer was further extracted with EtOAc (2×100 mL). The combined extracts were washed (brine), dried (MgSO₄) and concentrated. Purification of the residue by flash-chromatography (eluent: DCM/methanol: 8/2) gave the title compound. δ_{H} (DMSO): 3.35 (1H, m), 3.47 (1H, m), 3.95-4.05 (3H, m), 5.40 (1H, br s), 7.64-7.69 (3H, m), 7.86 (1H, d), 8.11-8.15 (2H, m); m/z (ES⁺)=317.05 [M+H]⁺; RT=2.50 min.

### Example 26

### 3-(6-Fluoronaphthalen-2-yl)-2-methyl-5,6-dihydroimidazo[2,1-b]thiazole hydrobromide

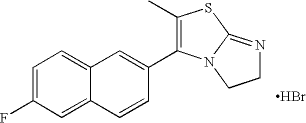

A stirred solution of 2-bromo-1-(6-fluoronaphthalen-2-yl)propan-1-one (Preparation 32, 0.86 g, 4.1 mmol) and imidazolidine-2-thione (0.42 g, 4.1 mmol) in EtOH (20 mL) was heated to reflux and AcOH (10 mL) was added. The reaction was stirred under reflux for 24 hr and then cooled to rt. Acetonitrile (20 mL) was added and a solid precipitated. The precipitate was collected by filtration to afford the title compound. δ_{H} DMSO): 2.31 (3H, s), 4.24 (2H, m), 4.35 (2H, m), 7.56 (1H, td), 7.69 (1H, d), 7.85 (1H, dd), 8.10 (1H, d) 8.15 (1H, m), 8.19 (1H, s), 9.50 (1H, br s); m/z (ES⁺)=285 [M+H]⁺; RT=2.56 min.

### Example 27

### 3-(6-Chloronaphthalen-2-yl)-2-methyl-5,6-dihydroimidazo[2,1-b]thiazole hydrobromide

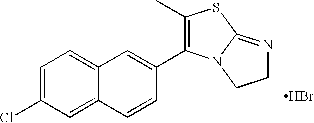

A stirred solution of 2-bromo-1-(6-chloronaphthalen-2-yl)propan-1-one (Preparation 36, 1.27 g, 4.3 mmol) and imidazolidine-2-thione (0.43 g, 4.3 mmol) in EtOH (20 mL) was heated to reflux and AcOH (10 mL) added. The reaction was stirred at reflux for 24 hr. The reaction was cooled to rt and the resulting precipitate was collected by filtration to afford the title compound. δ_{H} (DMSO): 2.31 (3H, s), 4.25 (2H, m), 4.35 (2H, m), 7.65 (1H, dd), 7.77 (1H, dd), 8.11 (2H, m), 8.18 (2H, s), 9.52 (1H, br s); m/z (ES⁺)=301 [M+H]⁺; RT=2.74 min.

### Example 28

### 2-Ethyl-3-naphthalen-2-yl-5,6-dihydroimidazo[2,1-b]thiazole hydrobromide

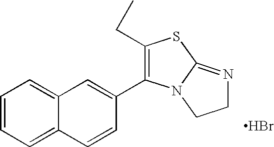

A stirred solution of 2-bromo-1-naphthalen-2-ylbutan-1-one (Preparation 38, 0.87 g, 3.5 mmol) and imidazolidine-2-thione (0.36 g, 3.5 mmol) in EtOH (10 mL) was heated to reflux and AcOH (5 mL) added. The reaction was stirred at reflux for 48 hr and then cooled to rt. The solvent was removed in vacuo, EtOAc and acetonitrile were added and a solid precipitated out. The solvent was removed in vacuo to give a solid which was triturated with Et₂O and then collected by filtration to afford the title compound. δ_{H} (DMSO): 1.17 (3H, t), 2.69 (2H, q), 4.24 (2H, m), 4.31 (2H, m), 7.64 (3H, m), 8.04 (2H, m), 8.11 (2H, m), 9.70 (1H, br s); m/z (ES⁺)=281 [M+H]⁺; RT=2.64 min.

### Example 29

### 3-(2-Methyl-5,6-dihydroimidazo[2,1-b]thiazol-3-yl)benzo[d]isothiazole hydrobromide

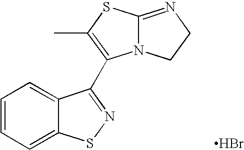

A stirred solution of 1-benzo[d]isothiazol-3-yl-2-bromopropan-1-one (Preparation 43, 0.4 g, 1.8 mmol) and imidazolidine-2-thione (0.18 g, 1.8 mmol) in EtOH (10 mL) was heated to reflux and AcOH (5 mL) was added. The reaction was heated under reflux for 48 hr, cooled to rt and the solvent removed in vacuo. EtOAc was added to the residue and precipitation was observed. The solid was collected by filtration to afford the title compound. δ_{H} (DMSO): 2.28 (3H, s), 4.24 (4H, m), 7.64 (1H, t), 7.74 (1H, t), 8.12 (1H, d), 8.39 (1H, d), 9.57 (1H, br s); m/z (ES⁺)=274 [M+H]⁺; RT=2.12 min.

### Example 30

### 3-(5-Chloronaphthalen-1-yl)-2-methyl-5,6-dihydroimidazo[2,1-b]thiazole hydrobromide

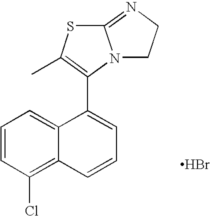

2-Bromo-1-(5-chloronaphthalen-1-yl)propan-1-one (Preparation 71, 118 mg, 0.4 mmol) and 2-imidazolidinethione (41 mg, 0.4 mmol) were heated to reflux in EtOH (10 mL) and AcOH (5 mL) for 24 hr. The reaction mixture was cooled to rt and concentrated in vacuo. EtOAc (20 mL) was added to the residue and the resulting precipitate was filtered affording the title compound. δ_{H} (DMSO): 2.05 (3H, s), 3.84 (1H, m), 4.07 (1H, m), 4.19 (2H, t), 7.62 (1H, t), 7.85 (4H, m), 8.43 (1H, d), 9.52 (1H, br s); RT=2.56 min; m/z (ES⁺)=301 [M+H]⁺.

### Example 31

### 3-Naphthalen-1-yl-2-propyl-5,6-dihydroimidazo[2,1-b]thiazole hydrobromide

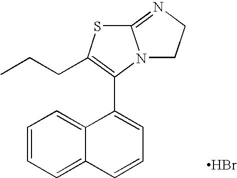

2-Bromo-1-naphthalen-1-ylpentan-1-one (Preparation 71, 1.57 g, 5.4 mmol) and 2-imidazolidinethione (0.55 g, 5.4 mmol) were heated to reflux in EtOH (20 mL) and AcOH (10 mL) for 16 hr. The reaction mixture was cooled to rt and concentrated in vacuo. EtOAc (20 mL) was added and the resulting precipitate was triturated with Et₂O and filtered, washed with Et₂O (2×20 mL) and dried under vacuum affording the title compound. δ_{H} (DMSO): 0.73 (3H, t), 1.44 (2H, m), 2.31 (1H, m), 2.43 (1H, m), 3.82 (1H, m), 4.05 (1H, m), 4.21 (2H, t), 7.67 (4H, m), 7.84 (1H, m), 8.08 (1H, m), 8.16 (1H, m), 9.71 (1H, br s); m/z (ES⁺)=295 [M+H]⁺; RT=2.69 min.

### Example 32

### 2-Methoxymethyl-3-naphthalen-1-yl-5,6-dihydroimidazo[2,1-b]thiazole

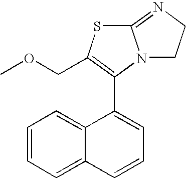

3-Benzyloxy-2-bromo-1-naphthalen-1-ylpropan-1-one (Preparation 65, 1.44 g, 3.9 mmol) and 2-imidazolidinethione (0.40 g, 3.9 mmol) were heated to reflux in AcOH (10 mL) and MeOH (20 mL) for 48 hr. The solvent was removed in vacuo and the residue partitioned between saturated NaHCO₃ solution (100 mL) and EtOAc (3×100 mL). The combined organic fractions were dried (MgSO₄), concentrated in vacuo and chromatographed on silica gel eluting with MeOH:DCM 1:49 to 3:97 affording the title compound. δ_{H} (CDCl₃): 3.45 (2H, m), 4.09 (2H, s), 4.18 (2H, m), 5.34 (3H, s), 7.47 (1H, m), 7.57 (3H, m), 7.95 (3H, m); m/z (ES⁺)=297 [M+H]⁺; RT=2.47 min.

### Example 33

### 3-(4-Chloronaphthalen-1-yl)-2-methyl-5,6-dihydroimidazo[2,1-b]thiazole hydrobromide

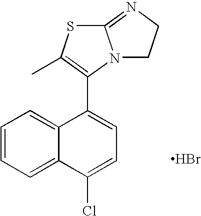

2-Bromo-1-(4-chloronaphthalen-1-yl)propan-1-one (Preparation 72, 6.05 g, 20.3 mmol) and 2-imidazolidinethione (2.08 g, 20.3 mmol) were heated to reflux in EtOH (100 mL) and AcOH (50 mL) for 24 hr. The reaction mixture was cooled to rt and concentrated in vacuo. Et₂O (100 mL) was added to the residue and this was stirred at rt for 30 min. The solid was filtered and washed with Et₂O (2×40 mL) and dried under vacuum affording the title compound. δ_{H} (DMSO): 2.06 (3H, s), 3.86 (1H, m), 4.09 (1H, m), 4.20 (2H, t), 7.69 (1H, d), 7.76 (1H, m), 7.83 (1H, m), 7.91 (2H, d), 8.34 (1H, d), 9.53 (1H, br s); m/z (ES⁺)=301 [M+H]⁺; RT=2.61 min.

### Example 34

### 2-Cyclopropyl-3-naphthalen-1-yl-5,6-dihydroimidazo[2,1-b]thiazole hydrobromide

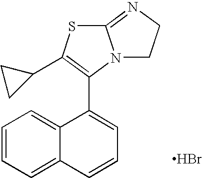

2-Bromo-2-cyclopropyl-1-naphthalen-1-ylethanone (Preparation 73, 282 mg, 0.98 mmol) and 2-imidazolidinethione (100 mg, 0.98 mmol) were heated to reflux in EtOH (10 mL) and AcOH (5 mL) for 16 hr. The reaction mixture was cooled to rt and concentrated in vacuo. The residue was partitioned between saturated NaHCO₃ solution (30 mL) and EtOAc (3×30 mL). The combined organic fractions were dried (MgSO₄), concentrated in vacuo and chromatographed on silica gel eluting with MeOH:DCM 7:93. The product was acidified with HBr (30% in AcOH) and the residue crystallised from MeCN and acetone affording the title compound. δ_{H} (DMSO): 0.51 (2H, m), 0.75 (2H, m), 1.69 (1H, m), 3.83 (1H, m), 4.09 (1H, m), 4.19 (2H, t), 7.65 (2H, m), 7.71 (2H, m), 7.89 (1H, m), 8.09 (1H, m), 8.17 (1H, d), 9.69 (1H, br s); m/z (ES⁺)=293 [M+H]⁺; RT=2.45 min.

### Example 35

### 3-(5-Chloronaphthalen-1-yl)-2-ethyl-5,6-dihydroimidazo[2,1-b]thiazole hydrobromide

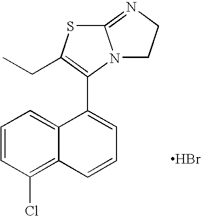

2-Bromo-1-(5-chloronaphthalen-1-yl)butan-1-one (Preparation 74, 0.43 g, 1.38 mmol) and 2-imidazolidinethione (0.14 g, 1.38 mmol) were heated to reflux in EtOH (10 mL) and AcOH (5 mL) for 16 hr. The reaction was cooled to rt and concentrated in vacuo. The residue was triturated with acetone (20 mL) and EtOAc (30 mL), and the filtered solid washed with EtOAc (20 mL), Et₂O (2×20 mL) and dried under vacuum affording the title compound. δ_{H} (DMSO): 1.03 (3H, t), 2.39 (2H, m), 3.82 (1H, m), 4.05 (1H, m), 4.20 (2H, t), 7.63 (1H, m), 7.84 (4H, m), 8.43 (1H, d), 9.66 (1H, br s); m/z (ES⁺)=315 [M+H]⁺; RT=2.79 min.

### Example 36

### 2-Isopropyl-3-naphthalen-2-yl-5,6-dihydroimidazo[2,1-b]thiazole

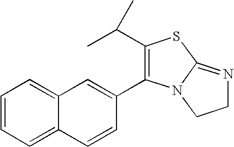

2-Bromo-3-methyl-1-naphthalen-2-ylbutan-1-one (Preparation 75, 0.70 g, 2.4 mmol) and 2-imidazolidinethione (0.24 g, 2.4 mmol) were heated to reflux in EtOH (10 mL) and AcOH (5 mL) for 16 hr. The reaction mix was cooled to rt and concentrated in vacuo. The residue was partitioned between saturated NaHCO₃ solution (5 mL) and EtOAc (3×20 mL). The combined organic fractions were dried (MgSO₄), concentrated in vacuo and chromatographed on silica gel eluting with MeOH:DCM 7:93 affording the title compound. δ_{H} (CDCl₃): 1.20 (6H, d), 3.04 (1H, m), 3.65 (2H, t), 4.15 (2H, t), 7.42 (1H, m), 7.56 (2H, m), 7.79 (1H, s), 7.90 (3H, m); m/z (ES⁺)=295 [M+H]⁺; RT=2.62 min.

### Example 37

### 3-(4-Fluoronaphthalen-1-yl)-2-isopropyl-5,6-dihydroimidazo[2,1-b]thiazole

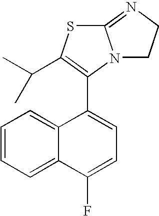

2-Bromo-1-(4-fluoronaphthalen-1-yl)-3-methylbutan-1-one (Preparation 76, 0.59 g, 1.9 mmol) and 2-imidazolidinethione (0.22 g, 2.1 mmol) were heated to reflux in EtOH (10 mL) and AcOH (5 mL) for 24 hr. The reaction mixture was cooled to rt and concentrated in vacuo. The residue was partitioned between saturated NaHCO₃ solution (50 mL) and EtOAc (3×20 mL). The combined organic fractions were dried (MgSO₄), concentrated in vacuo and chromatographed on silica gel eluting with MeOH:DCM 1:9 affording the title compound. δ_{H} (CDCl₃): 1.07 (3H, d), 1.18 (3H, d), 2.69 (1H, m), 3.47 (2H, m), 4.16 (2H, m), 7.22 (1H, m), 7.38 (1H, m), 7.65 (2H, m), 7.81 (1H, m), 8.21 (1H, m); m/z (ES⁺)=313 [M+H]⁺; RT=2.87 min.

### Example 38

### 3-(5-Chloronaphthalen-1-yl)-2-cyclopropyl-5,6-dihydroimidazo[2,1-b]thiazole hydrobromide

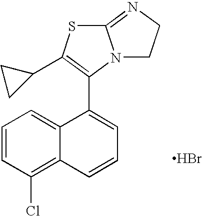

2-Bromo-1-(5-chloronaphthalen-1-yl)-2-cyclopropylethanone (Preparation 77, 140 mg, 0.43 mmol) and 2-imidazolidinethione (44 mg, 0.43 mmol) were heated to reflux in EtOH (6 mL) and AcOH (3 mL) for 24 hr. The reaction mixture was cooled to rt and concentrated in vacuo. The residue was triturated with Et₂O (20 mL), filtered and washed with Et₂O (2×10 mL) affording the title compound. δ_{H} (DMSO): 0.51 (2H, m), 0.75 (2H, m), 1.68 (1H, m), 3.83 (1H, m), 4.08 (1H, m), 4.17 (2H, m), 7.64 (1H, t), 7.88 (4H, m), 8.43 (1H, d), 9.65 (1H, br s); m/z (ES⁺)=327 [M+H]⁺; RT=2.74 min.

### Example 39

### 3-(8-Chloronaphthalen-2-yl)-2-methyl-5,6-dihydroimidazo[2,1-b]thiazole hydrobromide

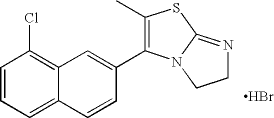

2-Bromo-1-(8-chloronaphthalen-2-yl)propan-1-one (Preparation 78, 222 mg, 0.75 mmol) and 2-imidazolidinethione (76 mg, 0.75 mmol) were heated to reflux in EtOH (10 mL) and AcOH (5 mL) for 16 hr. The reaction mixture was cooled to rt and concentrated in vacuo. EtOAc (20 mL) was added to the residue and this was filtered, washed with cold EtOAc (2×10 mL), Et₂O (2×10 mL) and dried under vacuum affording the title compound. δ_{H} (DMSO): 2.32 (3H, s), 4.25 (2H, m), 4.33 (2H, m), 7.65 (1H, t), 7.77 (1H, m), 7.83 (1H, d), 8.07 (1H, d), 8.24 (1H, d), 8.29 (1H, s), 9.55 (1H, br s); m/z (ES⁺)=301 [M+H]⁺; RT=2.77 min.

### Example 40

### 3-(4,5-Difluoronaphthalen-1-yl)-2-methyl-5,6-dihydroimidazo[2,1-b]thiazole hydrobromide

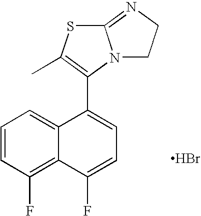

2-Bromo-1-(4,5-difluoronaphthalen-1-yl)propan-1-one (Preparation 93, 1.17 g, 3.91 mmol) and 2-imidazolidinethione (0.399 g, 3.91 mmol) were dissolved in an ethanol (10 mL)/acetic acid (5 mL) mixture and the reaction heated under reflux for 16 hr. The reaction mixture was cooled to rt and the solvent evaporated in vacuo. Trituration with acetonitrile yielded the title compound. δ_{H} (DMSO): 2.02 (3H, s), 3.80 (1H, q), 4.10 (1H, q), 4.2 (2H, m), 7.60-7.80 (5H, m); m/z (ES⁺)=303.07 [M+H]⁺; RT=2.45 min.

### Example 41

### 3-(4,5-Difluoronaphthalen-1-yl)-2-ethyl-5,6-dihydroimidazo[2,1-b]thiazole hydrobromide

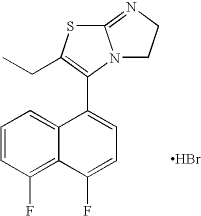

The title compound was prepared from 2-bromo-1-(4,5-difluoronaphthalen-1-yl)butan-1-one (Preparation 95, 1.1 g, 3.41 mmol) under similar conditions as described in Example 12. δ_{H} (DMSO): 1.00 (3H, t), 2.40 (2H, m), 3.80 (1H, q), 4.10 (1H, q), 4.20 (2H, m), 7.50-7.80 (5H, m), 9.60 (1H, br); m/z (ES⁺)=317.17 [M+H]⁺; RT=2.7 min.

### Example 42

### 3-(5,7-Dichloronaphthalen-1-yl)-2-methyl-5,6-dihydroimidazo[2,1-b]thiazole hydrobromide

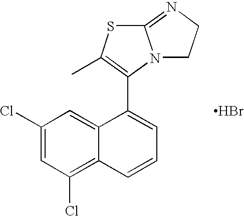

The title compound was prepared from 2-bromo-1-(5,7-dichloronaphthalen-1-yl)-propan-1-one (Preparation 99, 0.945 g, 2.84 mmol) and 2-imidazolidinethione under similar conditions as described in Example 12. δ_{H} (DMSO): 2.05 (3H, s), 3.85 (1H, q), 4.10 (1H, q), 4.20 (2H, t), 7.90 (3H, m), 8.00 (1H, d), 8.40 (1H, d), 9.50 (1H, br); m/z (ES⁺)=336.97 [M+H]⁺; RT=2.92 min.

### Examples 43-50

The procedure described in Example 1 was used to prepare the compounds of Examples 43-50.

| Ex | Structure | Name | LCMS | ¹H NMR data |
|---|---|---|---|---|
| 43 | 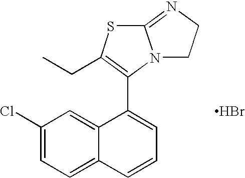 | 3-(7- Chloronaphthalen- 1-yl)-2-ethyl- 5,6- dihydroimidazo [2,1-b]thiazole hydrobromide | m/z (ES⁺) = 315.04 [M + H]⁺; RT = 2.79 min | δ_{H}(DMSO): 1.05 (3 H, t), 2.31-2.44 (2 H, m), 3.82-3.89 (1 H, m), 4.09- 4.16 (1 H, m), 4.21-4.26 (2 H, m), 7.67 (1 H, d), 7.71-7.79 (2 H, m), 7.97 (1 H, s), 8.16 (1 H, d), 8.23 (1 H, d), 9.80 (1 H, br s) |
| 44 | 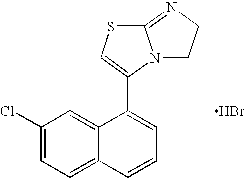 | 3-(7- Chloronaphthalen- 1-yl)-5,6- dihydroimidazo [2,1-b]thiazole hydrobromide | m/z (ES⁺) = 286.99 [M + H]⁺; RT = 2.56 min | δ_{H}(DMSO): 4.09-4.14 (2 H, m), 4.27-4.32 (2 H, m), 7.09 (1 H, s), 7.67- 7.73 (2 H, m), 7.79 (1 H, d), 8.11-8.16 (2 H, m), 8.21 (1 H, d), 9.67 (1 H, br s) |
| 45 | 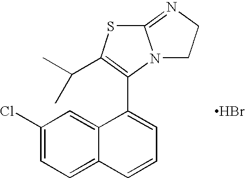 | 3-(7- Chloronaphthalen- 1-yl)-2- isopropyl-5,6- dihydroimidazo [2,1-b]thiazole hydrobromide | m/z (ES⁺) = 329.03 [M + H]⁺; RT = 2.92 min | δ_{H}(CDCl₃): 1.22 (3 H, d), 1.24 (3 H, d), 2.77- 2.82 (1 H, m), 3.89, 4.19, 4.40, 4.52 (4 H, 4m), 7.58-7.68 (4 H, m), 7.96 (1 H, d), 8.06 (1 H, d), 10.81 (1 H, br s) |
| 46 | 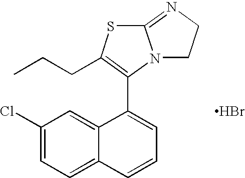 | 3-(7- Chloronaphthalen- 1-yl)-2-propyl- 5,6- dihydroimidazo [2,1-b]thiazole hydrobromide | m/z (ES⁺) = 329.02 [M + H]⁺; RT = 2.86 min | δ_{H}(CDCl₃): 0.89 (3 H, t), 1.57-1.62 (2 H, m), 2.38-2.48 (2 H, m), 3.92, 4.27, 4.39, 4.53 (4 H, 4m), 7.57-7.71 (4 H, m), 7.95 (1 H, d), 8.05 (1 H, d), 10.72 (1 H, br s) |
| 47 | 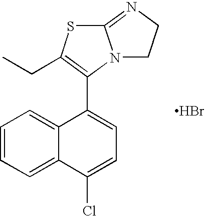 | 3-(4- Chloronaphthalen- 1-yl)-2-ethyl- 5,6- dihydroimidazo [2,1-b]thiazole hydrobromide | m/z (ES⁺) = 315.12 [M + H]⁺; RT = 2.77 min | δ_{H}(DMSO): 1.06 (3 H, t), 2.36-2.49 (2 H, m), 3.83-3.88 (1 H, m), 4.06- 4.11 (1 H, m), 4.20-4.24 (2 H, m), 7.70 (1 H, d), 7.77-7.80 (1 H, m) 7.83- 7.87 (1 H, m), 7.91-7.95 (2 H, m), 8.36 (1 H, d), 9.66 (1 H, br s) |
| 48 | 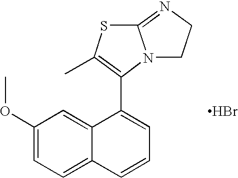 | 3-(7- Methoxynaphthalen- 1-yl)-2- methyl-5,6- dihydroimidazo [2,1-b]thiazole hydrobromide | m/z (ES⁺) = 297.09 [M + H]⁺; RT = 2.69 min | δ_{H}(DMSO): 2.09 (3 H, s), 3.89 (3 H, s), 3.92 (1 H, m), 4.17-4.23 (3 H, m), 7.05 (1 H, d), 7.32 (1 H, m), 7.53 (1 H, m), 7.65 (1 H, d), 8.02 (1 H, d), 8.09 (1 H, d), 9.59 (1 H, s) |
| 49 | 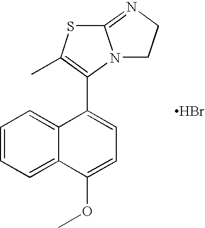 | 3-(4- Methoxynaphthalen- 1-yl)-2- methyl-5,6- dihydroimidazo [2,1-b]thiazole hydrobromide | m/z (ES⁺) = 297.07 [M + H]⁺; RT = 2.80 min | δ_{H}(DMSO): 2.06 (3 H, s), 3.83-3.90 (1 H, m), 4.06 (3 H, s), 4.10-4.13 (1 H, m), 4.20-4.25 (2 H, m), 7.16 (1 H, d), 7.62- 7.66 (3 H, m), 7.76 (1 H, d), 8.29 (1 H, d), 9.68 (1 H, br s) |
| 50 | 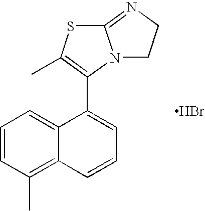 | 2-Methyl-3-(5- methyl- naphthalen-1-yl)- 5,6- dihydroimidazo [2,1-b]thiazole hydrobromide | m/z (ES⁺) = 281.01 [M + H]⁺; RT = 2.76 min | δ_{H}(DMSO): 2.07 (3 H, s), 2.74 (3 H, s), 3.84- 3.88 (1 H, m), 4.06 (1 H, m), 4.25 (2 H, m), 7.50 (2 H, m), 7.67-7.76 (3 H, m), 8.28 (1 H, d), 9.62 (1 H, br s) |

### Example 51

### 3-(5-Methoxynaphthalen-1-yl)-2-methyl-5,6-dihydroimidazo[2,1-b]thiazole hydrobromide

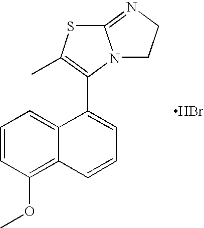

The title compound was prepared from 2-bromo-1-(5-methoxynaphthalen-1-yl)propan-1-one (Preparation 121) under similar conditions as described in Example 1. δ_{H} (DMSO): 2.1 (3H, s), 3.80 (1H, m), 4.05 (1H, m), 4.20 (2H, m), 7.08 (1H, d), 7.36 (1H, d), 7.57 (1H, m), 7.64 (2H, m), 8.40 (1H, m), 9.50 (1H, s); m/z (ES⁺)=297.08 [M+H]⁺; RT=2.70 min.

### Example 52

### 2-Methyl-3-(5-trifluoromethylnaphthalen-1-yl)-5,6-dihydro-imidazo[2,1-b]thiazole hydrochloride

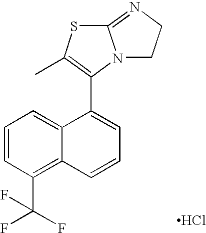

2-Bromo-1-(5-trifluoromethylnaphthalen-1-yl)propan-1-one (Preparation 130, 2.625 g, 7.93 mmol) and 2-imidazolidinethione (0.810 g, 7.93 mmol) were dissolved in ethanol (40 mL)/acetic acid (20 mL) mixture and the reaction heated under reflux for 16 hr. The reaction mixture was cooled to rt and the solvent evaporated in vacuo. The organics were basified with sat. NaHCO₃, extracted with DCM (3×50 mL), dried (MgSO₄) and concentrated in vacuo. Purification by flash-chromatography on silica gel (eluent: DCM/MeOH, 4/1) gave the free base which was acidified with cold ethereal 2M HCl (excess) to afford the title compound upon evaporation in vacuo. δ_{H} (DMSO): 2.10 (3H, s), 3.90 (1H, q), 4.10 (1H, q), 4.22 (2H, m), 7.80 (1H, t), 7.90 (1H, d), 7.95 (1H, t), 8.15 (1H, d), 8.20 (1H, d), 8.30 (1H, d), 11.00 (1H, br); m/z (ES⁺)=335.97 [M+H]⁺; RT=2.82 min.

### Example 53

### 3-(7-Fluoronaphthalen-1-yl)-2-methyl-5,6-dihydroimidazo[2,1-b]thiazole hydrobromide

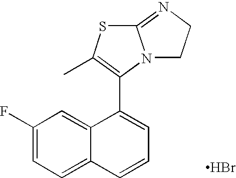

The title compound was prepared from 2-bromo-1-(7-fluoronaphthalen-1-yl)-propan-1-one (Preparation 133) and 2-imidazolidinethione under similar conditions as described in Example 1. δ_{H} (DMSO): 2.05 (3H, s), 3.90 (1H, q), 4.10 (1H, q), 4.20 (2H, t), 7.55-7.75 (4H, m), 8.05 (2H, m), 9.50 (1H, br); m/z (ES⁺)=285.05 [M+H]⁺; RT=2.55 min

### Example 54

### 3-(5-Fluoronaphthalen-1-yl)-2-methyl-5,6-dihydroimidazo[2,1-b]thiazole hydrobromide

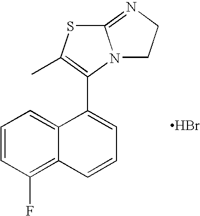

2-Bromo-1-(5-fluoronaphthalen-1-yl)propan-1-one (Preparation 150, 1.1 g, 3.96 mmol) and imidazolidine-2-thione (404 mg, 3.96 mmol) were dissolved in EtOH (15 mL) and AcOH (7.5 mL) and the reaction heated to reflux for 16 hr. The reaction mixture was cooled overnight and the precipitate collected by filtration, washed with acetonitrile (20 mL) to afford the title compound. δ_{H} (DMSO): 2.10 (3H, s), 3.85 (1H, m), 4.10 (1H, m), 4.20 (2H, m), 7.50 (1H, m), 7.70 (2H, m), 7.80 (2H, m), 8.25 (1H, m), 9.58 (1H, br s); m/z (ES⁺)=284.95 [M+H]⁺; RT=2.59 min.

### Examples 55-63

The procedure described in Example 1 was used to prepare the compounds of Examples 55-63.

| Ex | Structure | Name | LCMS | ¹H NMR data |
|---|---|---|---|---|
| 55 | 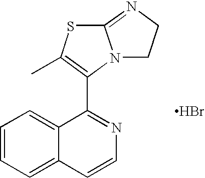 | 1-(2-Methyl-5,6- dihydro- imidazo [2,1-b]thiazol- 3-yl)- isoquinoline hydrobromide | m/z (ES⁺) = 268.00 [M + H]⁺; RT = 2.14 min | δ_{H}(DMSO): 2.13 (3 H, s), 4.01 (1 H, m), 4.19-4.35 (2 H, m), 4.43 (1 H, m), 7.82 (1 H, dd), 7.93 (1 H, dd), 8.06 (1 H, d), 8.11 (1 H, d), 8.18 (1 H, d), 8.72 (1 H, d), 9.63 (1 H, br s) |
| 56 | 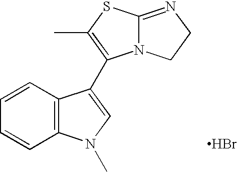 | 1-Methyl-3-(2- methyl-5,6- dihydroimidazo [2,1-b]thiazol- 3-yl)- 1H-indole- hydrobromide | m/z (ES⁺) = 270.01 [M + H]⁺; RT = 2.37 min | δ_{H}(DMSO): 2.24, 4.26 (6 H, 2s), 7.20 (1 H, dd), 7.31 (1 H, dd), 7.59 (2 H, 2d), 7.82 (1 H, s), 9.60 (1 H, br s) |
| 57 | 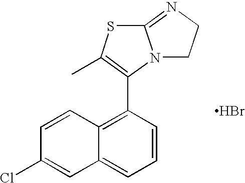 | 3-(6- Chloronaphthalen- 1-yl)-2-methyl-5,6- dihydro- imidazo [2,1-b]thiazole hydrobromide | m/z (ES⁺) = 301.04 [M + H]⁺; RT = 2.72 min | δ_{H}(DMSO): 2.07 (3 H, s), 3.87 (1 H, m), 4.11-4.25 (3 H, 2m), 7.64 (1 H, dd), 7.73-7.79 (2 H, m), 7.89 (1 H, d), 8.18 (1 H, d), 8.26 (1 H, d), 9.60 (1 H, br s) |
| 58 | 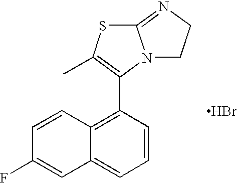 | 3-(6- Fluoronaphthalen- 1-yl)-2-methyl-5,6- dihydro- imidazo [2,1-b]thiazole hydrobromide | m/z (ES⁺) = 285.07 [M + H]⁺; RT = 2.42 min | δ_{H}(DMSO): 2.07 (3 H, s), 3.87 (1 H, m), 4.11-4.25 (3 H, 2m), 7.56 (1 H, m), 7.68 (1 H, d), 7.75 (1 H, dd), 7.91-7.96 (2 H, m), 8.18 (1 H, d), 9.61 (1 H, br s) |
| 59 | 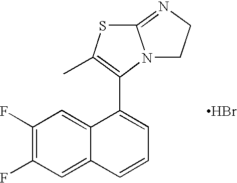 | 3-(6,7- Difluoronaphthalen- 1-yl)-2-methyl- 5,6-dihydro- imidazo [2,1-b]thiazole hydrobromide | m/z (ES⁺) = 3.03.03 [M + H]⁺; RT = 2.56 min | δ_{H}(DMSO): 2.06 (3 H, s), 3.87 (1 H, m), 4.09 (1 H, m), 4.20-4.25 (2 H, m), 7.72-7.77 (2 H, m) 7.93 (1 H, dd), 8.19-8.24 (2 H, m), 9.59 (1 H, br s) |
| 60 | 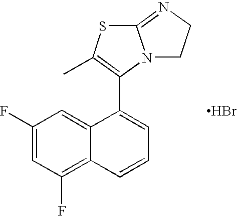 | 3-(5,7- Difluoronaphthalen- 1-yl)-2-methyl- 5,6-dihydro- imidazo [2,1-b]thiazole hydrobromide | m/z (ES⁺) = 303.08 [M + H]⁺; RT = 2.39 min | δ_{H}(DMSO): 2.07 (3 H, s), 3.88 (1 H, m), 4.10 (1 H, m), 4.20-4.25 (2 H, m), 7.57 (1 H, d) 7.67 (1 H, m), 7.79 (1 H, dd), 7.86 (1 H, d), 8.30 91 H, d), 9.57 (1 H, br s) |
| 61 | 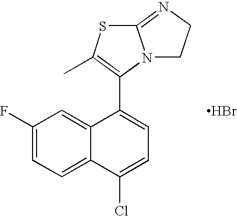 | 3-(4-Chloro-7- fluoro-naphthalen- 1-yl)-2-methyl-5,6- dihydroimidazo [2,1-b]thiazole hydrobromide | m/z (ES⁺) = 319.09 [M + H]⁺; RT = 2.81 min | δ_{H}(DMSO): 2.07 (3 H, s), 3.88 (1 H, m), 4.10 (1 H, m), 4.20-4.24 (2 H, m), 7.75-7.78 (3 H, m) 7.90 (1 H, d), 8.43 (1 H, dd), 9.58 (1 H, br s) |
| 62 | 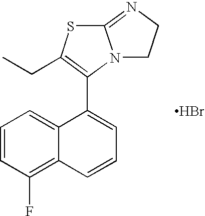 | 2-Ethyl-3-(5- fluoro-naphthalen- 1-yl)-5,6-dihydro- imidazo [2,1-b]thiazole hydrobromide | m/z (ES⁺) = 299.51 [M + H]⁺; RT = 2.57 min | δ_{H}(DMSO): 1.06 (3 H, t), 2.44 (2 H, m), 3.86, 4.08, (2 H, 2 m), 4.25 (2 H, m), 7.51 (1 H, dd) 7.63-7.68 (2 H, m), 7.71 (2 H, m), 8.31 (1 H, m), 9.71 (1 H, br s) |
| 63 | 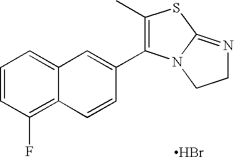 | 3-(5- Fluoronaphthalen- 2-yl)-2-methyl-5,6- dihydro- imidazo [2,1-b]thiazole hydrobromide | m/z (ES⁺) = 284.98 [M + H]⁺; RT = 2.62 min | δ_{H}(DMSO): 2.34 (3 H, t), 4.26, 4.37 (4 H, 2m), 7.52 (1 H, dd), 7.66 (1 H, m), 7.77 (1 H, dd), 7.94 (1 H, d), 8.23-8.27 (2 H, m), 9.58 (1 H, br s) |

### Example 64

### 3-(3,4-Dichlorophenyl)-2-methyl-5,6-dihydroimidazo[2,1-b]thiazole hydrobromide

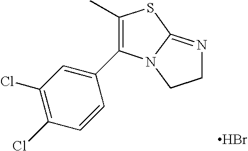

To a solution of 2-bromo-1-(3,4-dichlorophenyl)propan-1-one (Preparation 44, 1.50 g, 5.10 mmol) in EtOH (10.0 mL) and acetic acid (5.0 mL) was added imidazolidine-2-thione (530 mg, 5.10 mmol). The mixture was stirred at 110° C. under an inert atmosphere for 16 hr. The solvent was removed under reduced pressure and the resulting solid triturated with Et₂O (2×10 mL) then acetonitrile (1×5 mL) to afford the title compound. δ_{H} (DMSO): 9.50 (1H, br s), 7.90 (2H, m), 7.55 (1H, d), 4.30-4.20 (4H, m), 2.25 (3H, s); m/z (ES⁺)=284.92 [M−H]⁺; RT=2.36 min.

### Example 65

### 3-(3-Chloro-4-methylphenyl)-2-methyl-5,6-dihydroimidazo[2,1-b]thiazole hydrobromide

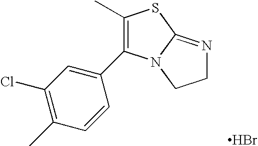

Prepared from 2-bromo-1-(4-chloro-3-methylphenyl)propan-1-one and imidazolidine-2-thione according to the method of Example 64. δ_{H} (DMSO): 9.50 (1H, br s), 7.60 (1H, d), 7.55 (1H, s), 7.40 (1H, t), 4.30-4.20 (4H, m), 2.40 (3H, s), 2.20 (3H, s); m/z (ES⁺)=265.01 [M−H]⁺; RT=2.65 min.

### Example 66

### 3-(4-Bromo-3-methylphenyl)-5,6-dihydroimidazo[2,1-b]thiazole hydrobromide

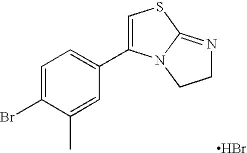

Imidazolidine-2-thione (0.2788 g, 2.73 mmol) was added to a solution of 2-bromo-1-(4-bromo-3-methylphenyl)ethanone (Preparation 47, 790 mg, 2.73 mmol) in EtOH (10 mL)/acetic acid (3 mL). The mixture was stirred at 110° C. under an inert atmosphere for 16 hr. The solvent was removed in vacuo and the resultant solid was washed with EtOH and Et₂O to yield the title compound. δ_{H} (MeOH): 7.68 (1H, d), 7.60 (1H, s), 7.40 (1H, d), 6.98 (1H, s), 4.60-4.40 (4H, m), 2.42 (3H, s); m/z (ES⁺)=296.01 [M−H]⁺; RT=2.31 min.

### Examples 67 to 70

The following compounds were made using procedures analogous to those described above:

### Example 67

### Cyclopropyl[3-(3,4-dichlorophenyl)-5,6-dihydroimidazo[2,1-b]thiazol-2-yl]methanol

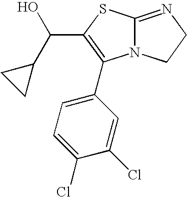

### Example 68

### [3-(4-Bromophenyl)-5,6-dihydroimidazo[2,1-b]thiazol-2-yl]methanol

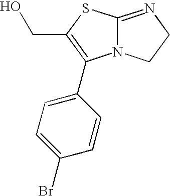

### Example 69

### 3-(4-Bromophenyl)-2-methyl-5,6-dihydroimidazo[2,1-b]thiazole hydrobromide

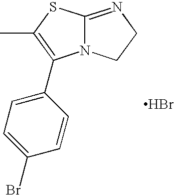

### Example 70

### 3-(4-Bromo-3-fluorophenyl)-2-methyl-5,6-dihydroimidazo[2,1-b]thiazole hydrobromide

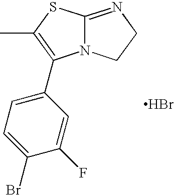

### Example 71

### 3-(4-Chloro-3-trifluoromethylphenyl)-2-isopropyl-5,6-dihydroimidazo[2,1-b]thiazole hydrobromide

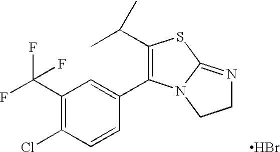

The title compound was prepared from 2-bromo-1-(4-chloro-3-trifluoromethylphenyl)-3-methylbutan-1-one (Preparation 146, 3.6 g, 10.48 mmol) and 2-imidazolidinethione under similar conditions as described in Example 1. δ_{H} (DMSO): 1.20 (6H, m), 3.00 (1H, m), 4.20 (4H, br s), 7.90 (1H, d), 8.00 (1H, d), 8.05 (1H, s), 9.70 (1H, br s); m/z (ES⁺)=346.98 [M+H]⁺; RT=2.74 min.

### Example 72

### 1-[3-(4-Chloro-3-trifluoromethylphenyl)-5,6-dihydroimidazo[2,1-b]thiazol-2-yl]ethanol

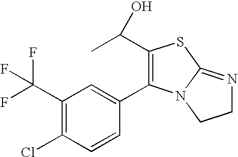

The title compound was prepared from 3-(4-chloro-3-trifluoromethylphenyl)-5,6-dihydroimidazo[2,1-b]thiazole-2-carbaldehyde (Preparation 144, 0.50 g, 1.503 mmol) under similar conditions as described in Example 64. δ_{H} (DMSO): 1.25 (3H, m), 3.50 (1H, m), 3.65 (1H, m), 4.00 (2H, m), 4.55 (1H, m), 5.40 (1H, m), 7.80 (1H, d), 7.85 (1H, d), 7.95 (1H, s); m/z (ES⁺)=348.93 [M+H]⁺; RT=2.40 min.

### Example 73

### 2-[3-(3,4-Dichlorophenyl)-5,6-dihydroimidazo[2,1-b]thiazol-2-yl]-propan-2-ol

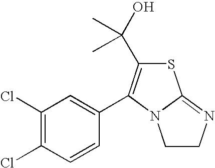

A solution of 3-(3,4-dichlorophenyl)-5,6-dihydroimidazo[2,1-b]thiazole-2-carboxylic acid methyl ester (Preparation 140, 1.50 g, 3.67 mmol) in THF (30.0 mL) was cooled at 0° C. under inert atmosphere and methylmagnesium bromide 2.0 M solution in diethyl ether (7.30 mL, 14.67 mmol) was added over 5 min. The mixture was stirred for 1 hr before removing the ice bath and stirred for further 16 hr. Saturated ammonium chloride (50 mL) was added slowly, stirred vigorously for 15 min then the obtained suspension filtered. The resulting solid was washed with water (2×5.0 mL) and dried under vacuo to afford the title compound. δ_{H} (DMSO): 1.25 (6H, s), 4.00 (2H, m), 4.15 (2H, m), 6.25 (1H, s), 7.55 (1H, d), 7.90 (2H, m); m/z (ES⁺)=328.94 [M+H]⁺; RT=2.34 min.

### Example 74

### 1-[3-(3,4-Dichlorophenyl)-5,6-dihydroimidazo[2,1-b]thiazol-2-yl]ethanol

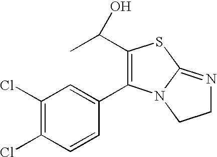

A solution of 3-(3,4-dichlorophenyl)-5,6-dihydroimidazo[2,1-b]thiazole-2-carbaldehyde (Preparation 138, 0.50 g, 1.67 mmol) in THF (20.0 mL) was cooled at 0° C. under inert atmosphere and methylmagnesium bromide 3.0 M solution in diethyl ether (1.67 mL, 5.01 mmol) was added over 5 min. The mixture was stirred for 1 hr before removing the ice bath and stirred for further 16 hr. Water (40 mL) was then added, stirred vigorously for 15 min then the aqueous layer extracted with EtOAc (3×50 mL), the organic layer dried (MgSO₄) and concentrated in vacuo. The yellow solid obtained was triturated with DCM (2×2.0 mL) and residual solvent removed in vacuo to the title compound. δ_{H} (DMSO): 1.25 (3H, d), 3.70-3.50 (2H, m), 4.00 (2H, m), 4.55 (1H, m), 5.40 (1H, m), 7.50 (1H, d), 7.80 (2H, m); m/z (ES⁺)=314.91 [M+H]⁺; RT=2.39 min.

### Example 75

### [3-(3,4-Dichlorophenyl)-5,6-dihydroimidazo[2,1-b]thiazol-2-yl]methanol

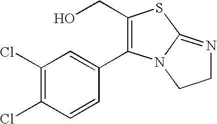

To a suspension of 3-(3,4-dichlorophenyl)-5,6-dihydroimidazo[2,1-b]thiazole-2-carbaldehyde (Preparation 138, 0.50 g, 1.672 mmol) in methanol (10.0 mL) at 0° C., was added sodium borohydride in one portion (95.0 mg, 2.51 mmol). The above mixture was stirred at 0° C. under an inert atmosphere for 2 hr before removing the cooling bath and then stirred for 16 hr. Water (40 mL) was then added, stirred vigorously for 1 hr then the obtained suspension filtered. The resulting solid was washed with Et₂O (2×10 mL) and dried under vacuo to afford the title compound. δ_{H} (DMSO): 3.65 (2H, m), 4.00 (2H, m), 4.20 (2H, m), 5.40 (1H, m), 7.50 (1H, d), 7.80 (2H, m); m/z (ES⁺)=300.94 [M+H]⁺; RT=2.29 min.

### Example 76

### 3-(3,4-Dichlorophenyl)-2-isopropyl-5,6-dihydroimidazo[2,1-b]thiazole hydrobromide

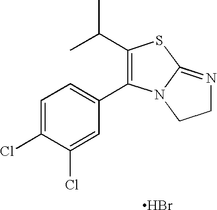

The title compound was prepared from 2-bromo-1-(3,4-dichlorophenyl)-3-methylbutan-1-one (Preparation 91, 1.6 g, 5.16 mmol) and 2-imidazolidinethione under similar conditions as described in Example 12. δ_{H} (d₄MeOH): 1.25 (6H, d), 3.10 (1H, m), 4.30 (4H, m), 7.40 (1H, d), 7.80 (2H, m); m/z (ES⁺)=314.97 [M+H]⁺; RT=2.72 min.

### Example 77

### 3-(4-Bromo-3-methylphenyl)-2-methyl-5,6-dihydroimidazo[2,1-b]thiazole hydrobromide

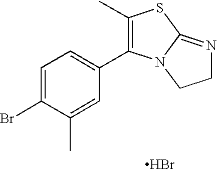

The title compound was prepared from 2-bromo-1-(4-bromo-3-methylphenyl)propan-1-one (Preparation 89, 2 g, 0.65 mmol) and 2-imidazolidinethione under similar conditions as described in Example 12. δ_{H} (d₄MeOH): 2.25 (3H, s), 2.50 (3H, s), 4.30 (4H, m), 7.20 (1H, d), 7.40 (1H, s), 7.80 (1H, d); m/z (ES⁺)=310.9 [M+H]⁺; RT=2.42 min.

### Example 78

### 3-(4-Bromo-2-fluorophenyl)-2-methyl-5,6-dihydroimidazo[2,1-b]thiazole hydrobromide

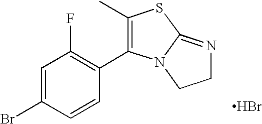

The title compound was prepared from 2-bromo-1-(4-bromo-2-fluorophenyl)propan-1-one (Preparation 87, 3.1 g, 100 mmol) and 2-imidazolidinethione under similar conditions as described in Example 12. δ_{H} (CDCl₃): 2.21 (3H, s), 4.30-4.65 (4H, m), 7.50 (3H, m); m/z (ES⁺)=314.88 [M+H]⁺; RT=2.36 min.

### Example 79

### 2-Methyl-3-(7,8-difluoronaphthalen-1-yl)-5,6-dihydroimidazo[2,1-b]thiazole hydrochloride

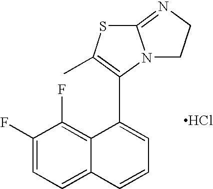

The title compound was prepared from 2-bromo-1-(7,8-difluoronaphthalene-1-yl)-propan-1-one (Preparation 84) under similar conditions as described in Example 64. δ_{H} (CDCl₃): 1.81 (3H, s), 3.35 (2H, m), 4.15 (2H, m), 7.39 (2H, m), 7.43 (1H, t), 7.61 (1H, m), 7.85 (1H, d); m/z⁺ (ES⁺)=302.94 [M+H]⁺; RT=2.54 min.

### Example 80

### 3-(4,5-Dichloronaphthalen-1-yl)-2-methyl-5,6-dihydroimidazo[2,1-b]thiazole hydrobromide

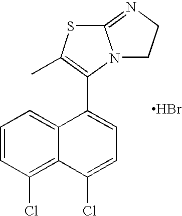

The title compound was prepared from 2-bromo-1-(4,5-dichloronaphthalen-1-yl)propan-1-one (Preparation 97, 0.73 g, 2.2 mmol) under similar conditions as described in Example 12. δ_{H} (DMSO): 2.00 (3H, s), 3.80 (1H, q), 4.10 (1H, q), 4.25 (2H, m), 7.60 (1H, t), 7.70 (1H, d), 7.90 (3H, m), 9.60 (1H, br); (ES⁺)=336.93 [M+H]⁺; RT=2.77 min.

### Example 81

### 3-(4-Hydroxynaphthalen-1-yl)-2-methyl-5,6-dihydroimidazo[2,1-b]thiazole

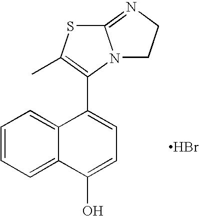

To a solution of 3-(4-methoxynaphthalen-1-yl)-2-methyl-5,6-dihydroimidazo[2,1-b]thiazole hydrobromide (Example 49, 0.207 g, 0.549 mmol) in DCM (10 mL) at 0° C. was added BBr₃ (1.0M, 1.64 mL) over a period of 2 min. After stirring for 16 hr at rt the reaction was partitioned between saturated sodium bicarbonate solution (100 mL) and EtOAc. The combined extracts were dried (MgSO₄) and concentrated to give an oil, which was dissolved in methanol and treated with HBr in acetic acid. The solvent was evaporated and the title compound was obtained via addition of EtOAc to precipitate the title compound which was collected by filtration. δ_{H} (CDCl₃): 2.13 (3H, s), 3.31 (1H, br s), 3.91-3.97 (1H, m), 4.09-4.15 (1H, m), 4.27-4.31 (2H, m), 6.96 (1H, d), 7.44 (1H, d), 7.51-7.63 (3H, m), 8.34 (1H, d); m/z (ES⁺)=283.09 [M+H]⁺; RT=2.49 min.

### Example 82

### 3-(7-Hydroxynaphthalen-1-yl)-2-methyl-5,6-dihydroimidazo[2,1-b]thiazole

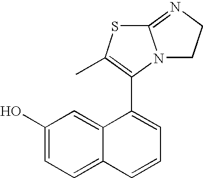

The title compound was prepared from 3-(7-methoxynaphthalen-1-yl)-2-methyl-5,6-dihydroimidazo[2,1-b]thiazole hydrobromide (Example 48) according to the procedure of Example 81. δ_{H} (DMSO): 1.88 (3H, s), 3.38-3.43 (2H, m), 3.90-4.03 (2H, m), 7.13-7.16 (2H, m), 7.34-7.38 (1H, m), 7.43-7.45 (1H, d), 7.87-7.92 (2H, m), 9.94 (1H, br s); m/z (ES⁺)=283.03 [M+H]⁺; RT=2.43 min.

### Example 83

### 3-(5-Chloronaphthalen-1-yl)-5,6-dihydroimidazo[2,1-b]thiazole hydrobromide

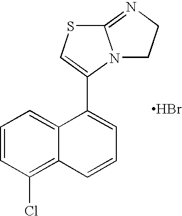

The title compound was prepared from 2-bromo-1-(5-chloro-naphthalen-1-yl)ethanone (Preparation 124) under similar conditions as described in Example 1. δ_{H} (DMSO): 4.04 (2H, m), 4.30 (2H, m), 7.64 (1H, m), 7.84 (3H, m), 8.03 (1H, d), 8.40 (1H, d), 9.61 (1H, s); m/z (ES⁺)=287.14 [M+H]⁺; RT=2.67 min.

### Example 84

### 2-Methylsulfanyl-3-(5,6,7,8-tetrahydronaphthalen-2-yl)-5,6-dihydroimidazo[2,1-b]thiazole hydrochloride

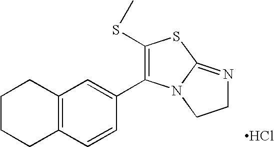

The title compound was prepared from 2-bromo-3-(5,6,7,8-tetrahydronaphthalen-2-yl)-5,6-dihydroimidazo[2,1-b]thiazole hydrobromide (Preparation 127) and dimethyl disulphide under similar conditions as described in Example 18. δ_{H} (DMSO): 1.79 (4H, m), 2.40 (3H, s), 2.81 (4H, m), 4.18-4.33 (4H, m), 727 (3H, m), 10.20 (1H, br); m/z (ES⁺)=303.04 [M+H]⁺; RT=2.79 min.

### Example 85

### 2-Ethyl-3-(7-fluoronaphthalen-1-yl)-5,6-dihydroimidazo[2,1-b]thiazole hydrobromide

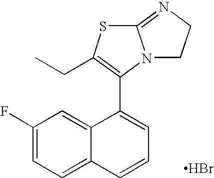

The title compound was prepared 2-bromo-1-(7-fluoronaphthalen-1-yl)butan-1-one and 2-imidazolidinethione (Preparation 135) under similar conditions as described in Example 1. δ_{H} (DMSO): 1.05 (3H, t), 2.30-2.50 (2H, m), 3.85 (1H, q), 4.05 (1H, q), 4.22 (2H, t), 7.55-7.75 (4H, m), 8.20-8.25 (2H, m), 9.60 (1H, br); m/z (ES⁺)=300.1 [M+H]⁺; RT=2.62 min.

### Example 86

### Cyclopropyl[3-(3,4-dichlorophenyl)-5,6-dihydroimidazo[2,1-b]thiazol-2-yl]-methanol

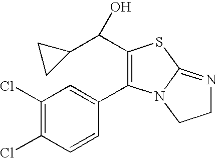

A solution of 3-(3,4-dichlorophenyl)-5,6-dihydroimidazo[2,1-b]thiazole-2-carbaldehyde (Preparation 138, 0.80 g, 2.67 mmol) in THF (30.0 mL) was cooled at 0° C. under inert atmosphere and cyclopropylmagnesium bromide 0.5 M solution in THF (16.0 mL, 8.01 mmol) was added over 10 min. The mixture was stirred for 1 hr before removing the ice bath and stirred for further 16 hr. Water (40 mL) was then added, stirred vigorously for 15 min then the aqueous layer extracted with EtOAc (3×50 mL), the organic layer dried (MgSO₄) and concentrated in vacuo. The yellow solid obtained was triturated with DCM (2×2.0 mL) and residual solvent removed in vacuo to afford the title compound. δ_{H} (DMSO): 0.10 (1H, m), 0.45-0.30 (3H, m), 1.00 (1H, m), 3.50 (1H, q), 3.75 (1H, q), 4.00 (3H, m), 5.40 (1H, m), 7.50 (1H, d), 7.75 (1H, d), 7.80 (1H, s); m/z (ES⁺)=340.90 [M+H]⁺; RT=2.56 min.

### Example 87

### 3-(4-Chloro-3-methylphenyl)-2-methyl-5,6-dihydroimidazo[2,1-b]thiazole hydrobromide

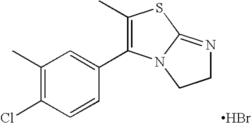

The title compound was prepared from 2-bromo-1-(4-chloro-3-methylphenyl)propan-1-one (Preparation 149, 5.15 g, 19.71 mmol) and 2-imidazolidinethione under similar conditions as described in Example 1. δ_{H} (DMSO): 2.21 (3H, s), 2.41 (1H, s), 4.20-4.35 (4H, m), 7.40 (1H, d), 7.55 (1H, s), 7.65 (1H, d), 9.45 (1H, br s); m/z (ES⁺)=264.99 [M+H]⁺; RT=2.39 min.

### Example 88

### 2-Allyl-3-(7-chloronaphthalen-1-yl)-5,6-dihydroimidazo[2,1-b]thiazole hydrobromide

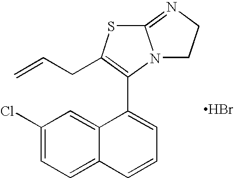

To a solution of 2-bromo-3-(7-chloronaphthalen-1-yl)-5,6-dihydroimidazo[2,1-b]thiazole hydrobromide (Preparation 151, 3.42 g) in THF (150 mL) under an inert atmosphere at 0° C. was added ethyl magnesium bromide solution (400% in ether, 7.65 mL) and stirred for 20 min. Allyl bromide (6.6 mL) was added dropwise, stirred at 0° C. for 20 min and allowed to warm to rt and stirred for 16 hr. Saturated ammonium chloride (40 mL) was added and the reaction mixture extracted into EtOAc (3×300 mL). The combined organic fractions were dried (MgSO₄) and concentrated in vacuo. Column chromatography (DCM:MeOH, 96:4) gave the desired product which was converted to the hydrobromide salt by treating a MeOH solution with 30% HBr in acetic acid and then filtration to afford the title compound. δ_{H} (DMSO): 3.18 (2H, m), 3.85 (1H, m), 4.10 (1H, m), 4.22 (2H, m), 5.80 (1H, m), 7.70 (3H, m), 8.01 (1H, s), 8.18 (1H, d), 8.25 (1H, d), 9.68 (1H, s); m/z (ES⁺)=327.05 [M+H]⁺; RT=2.97 min.

The biological activity of the compounds of the invention may be tested in the following assay systems:

### 1. [³H]Nisoxetine Binding to Noradrenaline Transporter Sites in Human Recombinant Membrane Preparation

### Membrane:

Membranes from a MDCK stable recombinant cell line expressing the human Noradrenaline Transporter sites was used to investigate the effects of compounds of the invention on binding of [³H]nisoxetine.

### Binding Assay:

In displacement experiments, membranes were incubated with [³H]nisoxetine at a single concentration of 1.0 nM and buffer (total binding) or test compound (10⁻⁶ M or a range of concentrations) or desipramine (1 μM; non-specific binding) for 90 min at 4° C.

Alternatively membranes were incubated with [³H]nisoxetine at a single concentration of 1.0 nM and buffer (total binding) or test compound (11 concentrations) or nisoxetine (2 μM, non-specific binding) for 4 hr at 4° C.

Membrane bound radioactivity was recovered by filtration. Filters were rapidly washed with ice-cold buffer and radioactivity determined by liquid scintillation counting.

2. [³H]Noradrenaline Incorporation into Rat Hypothalamus Synaptosomes Preparation of Synaptosomes

### [³H]Noradrenaline Incorporation Assay:

Rat hypothalamic synaptosomes prepared according to standard procedures were incubated with test compound (a range of concentrations) or protriptyline for 20 min at 37° C.

Synaptosomal incorporation was recovered by filtration. Filters were rapidly washed with ice-cold buffer and radioactivity determined by liquid scintillation counting.

### 3. [³H]Imipramine Binding to 5-HT Transporter Sites in Human Recombinant Membrane Preparation

### Membrane:

Membranes from a HEK-293 stable recombinant cell line expressing the human Serotonin Transporter sites was used to investigate the effects of compounds of the invention on binding of [³H]Imipramine.

### Binding Assay:

In displacement experiments, membranes were incubated with [³H]imipramine at a single concentration of 2.0 nM and buffer (total binding) or test compound (10⁻⁶ M or a range of concentrations) or imipramine (10 μM; non-specific binding) for 30 min at 22° C.

Alternatively binding was characterised using [³H]paroxetine. In these displacement experiments membranes were incubated with [³H]paroxetine at a single concentration of 0.5 nM and buffer (total binding) or test compound (11 concentrations) or paroxetine (2 μM, non-specific binding) for 4 hr at 4° C.

Membrane bound radioactivity was recovered by filtration. Filters were rapidly washed with ice-cold buffer and radioactivity determined by liquid scintillation counting.

4. [³H]Serotonin Incorporation into Rat Brain Synaptosomes Preparation of Synaptosomes

### [³H]-Serotonin Incorporation Assay:

Rat brain synaptosomes were incubated with test compound (a range of concentrations) or imipramine for 15 min at 37° C.

Synaptosomal incorporation was recovered by filtration. Filters were rapidly washed with ice-cold buffer and radioactivity determined by liquid scintillation counting.

### 5. 5HT_{1A} Binding Assay and [³⁵S]GTPγS Binding Assay in Human Recombinant Membrane Preparation

### Membrane:

Membranes from a HEK-293 stable recombinant cell line or a CHO-K1 stable recombinant cell line expressing the human Serotonin 1A receptor were used to investigate the effects of compounds of the invention on binding of [³H]8-OH-DPAT and the binding of [³⁵S]GTPγS.

### Binding Assay:

In displacement experiments, membranes were incubated with [³H]8-OH-DPAT at a single concentration of 0.5 nM and buffer (total binding) or test compound (10⁻⁶ M or a range of concentrations) or 8-OH-DPAT (10 μM; non-specific binding) for 60 min at 22° C.

Alternatively membranes were incubated with [³H]8-OH-DPAT at a single concentration of 1 nM and buffer (total binding) or test compound (11 concentrations) or 5-HT (2 μM; non-specific binding) for 60 min at 30° C.

Membrane bound radioactivity was recovered by filtration. Filters were rapidly washed with ice-cold buffer and radioactivity determined by liquid scintillation counting.

### Functional [³⁵]-GTPγS Binding Assay:

In [³⁵S]GTPγS binding assay, membranes were incubated with test compound (a range of concentrations) for 16 min at rt. Following this pre-incubation 150 pM of [³⁵S]GTPγS was added to the membrane and incubated for a further 45 min at 30° C. 5-HT and buspirone concentration effect curves were run alongside test compounds.

Membrane bound radioactivity was recovered by filtration. Filters were rapidly washed with ice-cold buffer and radioactivity determined by liquid scintillation counting.

Representative compounds of the invention exhibit displacements of >50% when measured at a concentration of 1 micromolar.

Examples 1-88 all exhibit 5-HT_{1A} agonism and noradrenaline reuptake inhibition or 5-HT_{1A} agonist noradrenaline reuptake inhibition and 5-HT reuptake inhibition in these assay systems.

The biological activity of the compounds of the invention may also be tested in in vivo models known to those skilled in the art. Thus, for example, representative compounds of the invention following acute oral dosing of lean male Sprague Dawley rats or female Wistar rats significantly reduced food intake for up to 24 hr compared to controls to a greater degree than sibutramine. Sub-chronic oral administration of representative compounds significantly attenuated weight gain in a diet-induced obese mouse model over 21 days and sub-chronic oral dosing once daily to high-fat fed male Sprague Dawley rats for 21 days reduced weight gain and to a greater extent than sibutramine.

Representative compounds have also demonstrated effects including decrease in fat pad masses and/or decrease in plasma levels of leptin, glucose, insulin or triglycerides as compared to vehicle-treated controls after sub-chronic oral dosing in rats. Also, in contrast to sibutramine, representative compounds of the invention showed no increases in heart rate or mean arterial blood pressure in conscious, telemeterised normotensive rats at doses significantly higher than those which give efficacy.

What is claimed is:

1. A compound of formula (I): 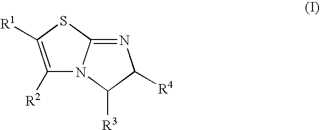
or a pharmaceutically acceptable salt thereof,
wherein R¹ is hydrogen, halo, C₁₋₆ alkyl optionally substituted by one or more halo atoms or hydroxy groups, C₃₋₆ cycloalkyl optionally substituted by one or more halo atoms or hydroxy groups, C₁₋₂ alkylC₃₋₆ cycloalkyl optionally substituted by one or more halo atoms or hydroxy groups, C₁₋₆ alkoxycarbonyl, cyano, —C═N—OR⁷, C₂₋₆ alkenyl optionally substituted by one or more halo atoms or hydroxy groups in which hydroxy is not directly attached to either carbon of the double bond, C₂₋₆ alkynyl optionally substituted by one or more halo atoms or hydroxy groups in which hydroxy is not directly attached to either carbon of the triple bond, (CH₂)ₘNR⁵R⁶, C₁₋₃alkoxy, C₁₋₃ alkylthio, C₁₋₃ alkoxyC₁₋₃ alkyl or C₁₋₃ alkylthioC₁₋₃ alkyl;
R² is naphthalen-1-yl, naphthalen-2-yl, thieno[2,3-b]thiophen-2-yl, quinolin-2-yl, isoquinolinyl or benzoisothiaxol-3-yl;
R² may be optionally substituted by one or more groups selected from halo, cyano, hydroxy, NR⁵R⁶, CONR⁵R⁶, or COOR⁷, or C₁₋₃ alkyl, C₂₋₃ alkenyl, C₂₋₃ alkynyl, C₃₋₆ cycloalkyl, C₁₋₃ alkoxy, C₁₋₃ hydroxyalkyl, C₂₋₃ alkoxyalkyl or C₁₋₃ alkylS(O)ₙ any of which may be optionally substituted by one or more halo atoms;
R³ and R⁴ are independently hydrogen or C₁₋₃ alkyl;
R⁵ and R⁶ are independently hydrogen or C₁₋₃ alkyl, or together with the nitrogen to which they are attached form a 5- or 6-membered heterocyclyl group;
R⁷ is hydrogen or C₁₋₃ alkyl;
m is 1, 2 or 3; and
n is 0, 1 or 2;
provided that the compound is not:
a) 3-naphthalen-1-yl-5,6-dihydroimidazo[2,1-b]thiazole hydrobromide, or
b) 3-naphthalen-2-yl-5,6-dihydroimidazo[2,1-b]thiazole hydrobromide.

2. A compound according to claim 1 wherein R¹ is hydrogen, C₁₋₆ alkyl optionally substituted by one or more halo atoms or hydroxy groups, C₃₋₆ cycloalkyl optionally substituted by one or more halo atoms or hydroxy groups, or C₁₋₂ alkylC₃₋₆ cycloalkyl optionally substituted by one or more halo atoms or hydroxy groups.

3. A compound according to claim 2 wherein R¹ is C₁₋₆ alkyl.

4. A compound according to claim 1 wherein R² is naphthalen-1-yl.

5. A compound according to claim 1 wherein R² is substituted by one or two substituents selected from halo and C₁₋₃ alkyl.

6. A compound according to claim 1 wherein R² is naphthalen-1-yl which is unsubstituted or substituted in one or two of the 4-, 5- or 7-positions by halo.

7. A compound according to claim 6 wherein R² is naphthalen-1-yl substituted in one or two of the 4-, 5- or 7-positions by fluoro or chloro.

8. A compound according to claim 1 wherein R³ and R⁴ are both hydrogen.

9. A compound selected from
2-Methyl-3-naphthalen-2-yl-5,6-dihydroimidazo[2,1-b]thiazole;
2-Bromo-3-naphthalen-2-yl-5,6-dihydroimidazo[2,1-b]thiazole;
2-Chloro-3-naphthalen-1-yl-5,6-dihydroimidazo[2,1-b]thiazole;
3-Thieno[2,3-b]thiophen-2-yl-5,6-dihydroimidazo[2,1-b]thiazole;
3-(4-Methylnaphthalen-1-yl)-5,6-dihydroimidazo[2,1-b]thiazole;
2-(5,6-Dihydroimidazo[2,1-b]thiazol-3-yl)quinoline;
3-(4-Fluoronaphthalen-1-yl)-5,6-dihydroimidazo[2,1-b]thiazole;
3-Naphthalen-2-yl-5,6-dihydroimidazo[2,1-b]thiazole-2-carboxylic acid ethyl ester,
2-Bromo-3-naphthalen-1-yl-5,6-dihydroimidazo[2,1-b]thiazole;
2-Methyl-3-naphthalen-1-yl-5,6-dihydroimidazo[2,1-b]thiazole;
3-Naphthalen-1-yl-5,6-dihydroimidazo[2,1-b]thiazol-2-yl methanol;
2-Ethynyl-3-naphthalen-1-yl-5,6-dihydroimidazo[2,1-b]thiazole;
3-(7-Chloronaphthalen-1-yl)-2-methyl-5,6-dihydroimidazo[2,1-b]thiazole;
1-(3-Naphthalen-1-yl-5,6-dihydroimidazo[2,1-b]thiazol-2-yl)ethanol;
3-Naphthalen-1-yl-5,6-dihydroimidazo[2,1-b]thiazole-2-carbonitrile;
2-Methylsulfanyl-3-naphthalen-1-yl-5,6-dihydroimidazo[2,1-b]thiazole;
(3-Naphthalen-2-yl-5,6-dihydroimidazo[2,1-b]thiazol-2-yl)methanol;
1-(3-Naphthalen-2-yl-5,6-dihydroimidazo[2,1-b]thiazol-2-yl)ethanol;
2-(3-Naphthalen-2-yl-5,6-dihydroimidazo[2,1-b]thiazol-2-yl)propan-2-ol;
3-(4-Fluoronaphthalen-1-yl)-2-methyl-5,6-dihydroimidazo[2,1-b]thiazole;
2-Ethyl-3-naphthalen-1-yl-5,6-dihydroimidazo[2,1-b]thiazole;
2-Isopropyl-3-naphthalen-1-yl-5,6-dihydroimidazo[2,1-b]thiazole;
[3-(7-Chloronaphthalen-1-yl)-5,6-dihydroimidazo[2,1-b]thiazol-2-yl]methanol;
3-(6-Fluoronaphthalen-2-yl)-2-methyl-5,6-dihydroimidazo[2,1-b]thiazole;
3-(6-Chloronaphthalen-2-yl)-2-methyl-5,6-dihydroimidazo[2,1-b]thiazole;
2-Ethyl-3-naphthalen-2-yl-5,6-dihydroimidazo[2,1-b]thiazole;
3-(2-Methyl-5,6-dihydroimidazo[2,1-b]thiazol-3-yl)benzo[d]isothiazole;
3-(5-Chloronaphthalen-1-yl)-2-methyl-5,6-dihydroimidazo[2,1-b]thiazole;
3-Naphthalen-1-yl-2-propyl-5,6-dihydroimidazo[2,1-b]thiazole;
2-Methoxymethyl-3-naphthalen-1-yl-5,6-dihydroimidazo[2,1-b]thiazole;
3-(4-Chloronaphthalen-1-yl)-2-methyl-5,6-dihydroimidazo[2,1-b]thiazole;
2-Cyclopropyl-3-naphthalen-1-yl-5,6-dihydroimidazo[2,1-b]thiazole;
3-(5-Chloronaphthalen-1-yl)-2-ethyl-5,6-dihydroimidazo[2,1-b]thiazole;
2-Isopropyl-3-naphthalen-2-yl-5,6-dihydroimidazo[2,1-b]thiazole;
3-(4-Fluoronaphthalen-1-yl)-2-isopropyl-5,6-dihydroimidazo[2,1-b]thiazole;
3-(5-Chloronaphthalen-1-yl)-2-cyclopropyl-5,6-dihydroimidazo[2,1-b]thiazole;
3-(8-Chloronaphthalen-2-yl)-2-methyl-5,6-dihydroimidazo[2,1-b]thiazole;
3-(4,5-Difluoronaphthalen-1-yl)-2-methyl-5,6-dihydroimidazo[2,1-b]thiazole;
3-(4,5-Difluoronaphthalen-1-yl)-2-ethyl-5,6-dihydroimidazo[2,1-b]thiazole;
3-(5,7-Dichloronaphthalen-1-yl)-2-methyl-5,6-dihydroimidazo[2,1-b]thiazole;
3-(7-Chloronaphthalen-1-yl)-2-ethyl-5,6-dihydroimidazo[2,1-b]thiazole;
3-(7-Chloronaphthalen-1-yl)-5,6-dihydroimidazo[2,1-b]thiazole;
3-(7-Chloronaphthalen-1-yl)-2-isopropyl-5,6-dihydroimidazo[2,1-b]thiazole;
3-(7-Chloronaphthalen-1-yl)-2-propyl-5,6-dihydroimidazo[2,1-b]thiazole;
3-(4-Chloronaphthalen-1-yl)-2-ethyl-5,6-dihydroimidazo[2,1-b]thiazole;
3-(7-Methoxynaphthalen-1-yl)-2-methyl-5,6-dihydroimidazo[2,1-b]thiazole;
3-(4-Methoxynaphthalen-1-yl)-2-methyl-5,6-dihydroimidazo[2,1-b]thiazole;
2-Methyl-3-(5-methyl-naphthalen-1-yl)-5,6-dihydroimidazo[2,1-b]thiazole;
3-(5-Methoxynaphthalen-1-yl)-2-methyl-5,6-dihydroimidazo[2,1-b]thiazole;
2-Methyl-3-(5-trifluoromethylnaphthalen-1-yl)-5,6-dihydroimidazo[2,1-b]thiazole;
3-(7-Fluoronaphthalen-1-yl)-2-methyl-5,6-dihydroimidazo[2,1-b]thiazole;
3-(5-Fluoronaphthalen-1-yl)-2-methyl-5,6-dihydroimidazo[2,1-b]thiazole;
1-(2-Methyl-5,6-dihydroimidazo[2,1-b]thiazol-3-yl)isoquinoline;
1-Methyl-3-(2-methyl-5,6-dihydroimidazo[2,1-b]thiazol-3-yl)-1H-indole;
3-(6-Chloronaphthalen-1-yl)-2-methyl-5,6-dihydroimidazo[2,1-b]thiazole;
3-(6-Fluoronaphthalen-1-yl)-2-methyl-5,6-dihydroimidazo[2,1-b]thiazole;
3-(6,7-Difluoronaphthalen-1-yl)-2-methyl-5,6-dihydroimidazo[2,1-b]thiazole;
3-(5,7-Difluoronaphthalen-1-yl)-2-methyl-5,6-dihydroimidazo[2,1-b]thiazole;
3-(4-Chloro-7-fluoro-naphthalen-1-yl)-2-methyl-5,6-dihydroimidazo[2,1-b]thiazole;
2-Ethyl-3-(5-fluoro-naphthalen-1-yl)-5,6-dihydroimidazo[2,1-b]thiazole;
3-(5-Fluoronaphthalen-2-yl)-2-methyl-5,6-dihydroimidazo[2,1-b]thiazole;
2-Methyl-3-(7,8-difluoronaphthalen-1-yl)-5,6-dihydroimidazo[2,1-b]thiazole;
3-(4,5-Dichloronaphthalen-1-yl)-2-methyl-5,6-dihydroimidazo[2,1-b]thiazole;
3-(4-Hydroxynaphthalen-1-yl)-2-methyl-5,6-dihydroimidazo[2,1-b]thiazole;
3-(7-Hydroxynaphthalen-1-yl)-2-methyl-5,6-dihydroimidazo[2,1-b]thiazole;
3-(5-Chloronaphthalen-1-yl)-5,6-dihydroimidazo[2,1-b]thiazole;
2-Ethyl-3-(7-fluoronaphthalen-1-yl)-5,6-dihydroimidazo[2,1-b]thiazole; and
2-Allyl-3-(7-chloronaphthalen-1-yl)-5,6-dihydroimidazo[2,1-b]thiazole;
as the free base or a pharmaceutically acceptable salt thereof.

10. A pharmaceutical composition comprising a compound according to claim 1, or a pharmaceutically acceptable salt thereof and a pharmaceutically acceptable carrier.

11. A method for the treatment of a disease or condition in which Noradrenaline and optionally also Serotonin reuptake plays a role comprising a step of administering to a subject in need thereof an effective amount of a compound of formula (I′): 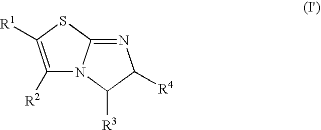
or a pharmaceutically acceptable salt thereof,
wherein R¹ is hydrogen, halo, C₁₋₆ alkyl optionally substituted by one or more halo atoms or hydroxy groups, C₃₋₆ cycloalkyl optionally substituted by one or more halo atoms or hydroxy groups, C₁₋₂ alkylC₃₋₆ cycloalkyl optionally substituted by one or more halo atoms or hydroxy groups, C₁₋₆ alkoxycarbonyl, cyano, —C═N—OR⁷, C₂₋₆ alkenyl optionally substituted by one or more halo atoms or hydroxy groups in which hydroxy is not directly attached to either carbon of the double bond, C₂₋₆ alkynyl optionally substituted by one or more halo atoms or hydroxy groups in which hydroxy is not directly attached to either carbon of the triple bond, (CH₂)ₘNR⁵R⁶, C₁₋₃alkoxy, C₁₋₃ alkylthio, C₁₋₃ alkoxyC₁₋₃ alkyl or C₁₋₃ alkylthioC₁₋₃ alkyl;
R² is an 8- to 10-membered bicyclic aromatic group containing up to 3 heteroatoms selected from N and S, provided that R² is not benzo[b]thiophene;
R² may be optionally substituted by one or more groups selected from halo, cyano, hydroxy, NR⁵R⁶, CONR⁵R⁶, or COOR⁷, or C₁₋₃ alkyl, C₂₋₃ alkenyl, C₂₋₃ alkynyl, C₃₋₆ cycloalkyl, C₁₋₃ alkoxy, C₁₋₃ hydroxyalkyl, C₂₋₃ alkoxyalkyl or C₁₋₃ alkylS(O)ₙ any of which may be optionally substituted by one or more halo atoms;
R³ and R⁴ are independently hydrogen or C₁₋₃alkyl;
R⁵ and R⁶ are independently hydrogen or C₁₋₃ alkyl, or together with the nitrogen to which they are attached form a 5- or 6-membered heterocyclyl group;
R⁷ is hydrogen or C₁₋₃alkyl;
m is 1, 2 or 3; and
n is 0, 1 or 2.

12. A method for the treatment of a disease or condition in which Noradrenaline and optionally also Serotonin reuptake plays a role and in which 5-HT_{1A} agonism is desirable comprising a step of administering to a subject in need thereof an effective amount of a compound of formula (I′) as defined in claim 11, or a pharmaceutically acceptable salt thereof.

13. A method for the regulation of food intake and/or satiety comprising a step of administering to a subject in need thereof an effective amount of a compound of formula (I′) as defined in claim 11, or a pharmaceutically acceptable salt thereof.

14. A method for the treatment of obesity comprising a step of administering to a subject in need thereof an effective amount of a compound of formula (I′) as defined in claim 11, or a pharmaceutically acceptable salt thereof.

15. A method for the treatment of a metabolic disease selected from Type II diabetes, metabolic syndrome (syndrome X), impaired glucose tolerance, dyslipidemia, hyperlipidemia, hypertriglyceridemia, hypercholesterolemia, low HDL levels and hypertension, comprising a step of administering to a subject in need thereof an effective amount of a compound of formula (I′) as defined in claim 11, or a pharmaceutically acceptable salt thereof.

16. A method for reducing the potential for cardiovascular side effects in the treatment of a disease or condition as defined in claim 14 comprising a step of administering to a subject in need thereof an effective amount of a compound of formula (I′) as defined in claim 11, or a pharmaceutically acceptable salt thereof.

17. A process for the production of a compound of formula (I) which comprises the step of reacting a compound of formula (III): 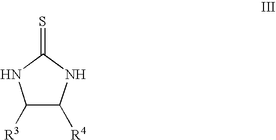
with a compound of formula (IV): 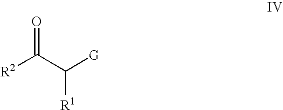
wherein R¹ to R⁴ are as defined in claim 1 and G is hydrogen or a leaving group.

18. A compound of formula (II): 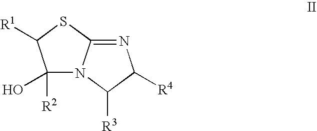
wherein R¹, R², R³ and R⁴ are as defined in claim 1.

19. A compound of formula (X): 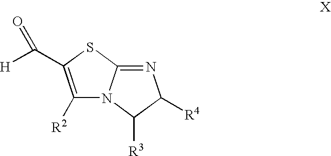
wherein R², R³ and R⁴ are as defined in claim 1.

20. A method for the treatment of obesity comprising a step of administering to a subject in need thereof an effective amount of a compound according to claim 1, including the compounds of provisos a) and b), or a pharmaceutically acceptable salt thereof.
